(19) United States
(12) Patent Application Publication
(10) Pub. No.: US 2009/0005258 A1
(43) Pub. Date: Jan. 1, 2009

(54) Diagnosis of Metastases in Hnscc Tumours

(76) Inventors: Frank Charles P. Holstege, Houten (NL); Pieter Johannes Slootweg, Bennekom (NL); Paul Roepman, Wageningen (NL)

Correspondence Address: MORRISON & FOERSTER LLP, 12531 HIGH BLUFF DRIVE, SUITE 100, SAN DIEGO, CA, 92130-2040, US

(21) Appl. No.: 11/813,510

(22) PCT Filed: Jan. 6, 2006
(86) PCT No.: PCT/NL2006/000005

§ 371 Date: Jul. 28, 2008

(30) Foreign Application Priority Data
Jan. 6, 2005 (EP) 05075017.3

(51) Int. Cl. C40B 30/04; C40B 40/06; G06F 19/00; C40B 50/00

(52) U.S. Cl. 506/9; 506/16; 506/23; 702/20

(57) ABSTRACT

The invention relates to the detection or prediction of metastases of head and neck squamous cell carcinoma (HNSCC) with the use of gene expression profiles. A gene signature has been identified which is able to detect or predict the occurrence of these metastases better than current clinical methods. Part of the invention are micro-arrays comprising this signature and methods for performing the detection and/or prediction.

### FIELD OF THE INVENTION

The invention relates to the field of tumour diagnosis, in particular to predict the existence of metastases of a tumour, more in particular to the detection of lymph node metastases of head and neck squamous cell carcinoma (HNSCC) especially those that arise in the oral cavity and oropharynx.

### STATE OF THE PRIOR ART

Head and neck squamous cell carcinoma (HNSCC) consists of a heterogenous group of neoplasms that arise from the epithelium of the upper aero-digestive tract. HNSCC is the fifth most common malignancy in humans and is particularly frequent in regions where alcohol and tobacco use is high (Sankanarayanan, R. et al., (1998) Anticancer Res. 18, 4779-4786). The survival rate of patients with cervical lymph node metastases is reduced by almost 50% (Jones, A. S. et al., (1994) Clin. Otolaryngol. 19, 63-69; Hahn, S. S. et al., (1987) J. Radiat. Oncol. Biol. Phys. 13, 1143-1147). As with most forms of cancer, treatment depends largely on progression stage (Forastiere, A. et al., (2001) N. Eng. J. Med. 345, 1890-1900).

Metastasis is the process whereby cancers spread to distinct sites in the body. It is the principal cause of death in individuals suffering from cancer. For some tumor types, the earliest detectable sign of metastasis is the presence of malignant cells in lymph nodes close to the site of the primary tumour. Early detection of local lymph node metastases is currently pivotal for appropriate treatment of many types of cancer. However, because of difficulties in detecting lymph node metastases reliably, many patients currently receive inappropriate treatment.

Most patients with HNSCC, especially those in the oral cavity or oropharynx have the primary tumour removed. Treatment of clinically diagnosed lymph node metastasis positive patients (N+) involves the additional surgical removal of a significant portion of the neck, including all five local lymph node levels: radical neck dissection (RND) (Robbins, K. T. et al., (2002) Arch. Otolaryngol. Head Neck Surg. 128, 751-758). However, after histological examination of the removed tissue, approximately 10-20% of the clinically diagnosed N+ patients appear to be N0 (metastasis free) (Woolgar, J. A., (1999) Br. J. Oral Maxillofac. Surg. 37, 205-209). Clinical diagnosis of N0 lymph node status is even less accurate. Histological examination of electively operated clinically diagnosed N0 patients reveals that about one-third have positive neck nodes (Jones, A. S. et al., (1993) Eur. Arch. Otorhinolaryngol. 250, 446-449). Different strategies exist for neck treatment of N0 diagnosed patients (Pillsbury, H. C., et al., (1997) Laryngoscope 107, 1294-1315). One is the so-called “watch and wait” strategy by which N0 diagnosed patients do not undergo any neck dissection. The involves the risk of fatality by allowing overlooked metastases to develop and spread further. Since the prevalence of false-negative predictions is very high, most clinics perform neck surgery for all diagnosed N0 patients. In this case most often a supra-omohyoidal neck dissection (SOHND) is performed, removing the three upper lymph node levels (Robbins, K. T. et al., supra). This treatment is less appropriate than an RND for those N+ patients falsely diagnosed as N0 and, moreover, completely unnecessary for all patients correctly diagnosed as N0. Although SOHND is less rigorous than RND, the treatment cause disfigurement, long-term discomfort, pain and can lead to additional complications such as shoulder disability (e.g. Short, S. O. et al., (1984) Am. J. Surg. 148, 478-482). Both treatments strategies result in over- or undertreatment due to limitations in detecting lymph node metastasis reliably.

DNA microarray based gene expression profiling has previously been shown to be useful for cancer classification (e.g. Valk, P. J. et al., (2004) N. Eng. J. Med. 350, 1605-1616) and prognosis-based treatment (Van 't Veer, L. J. et al., (2002) Nature 415, 530-536 and WO 2004/065545). Gene expression profiling has revealed genes that are differentially regulated in metastases (Clark, E. A. et al., (2000) Nature 406, 532-535; Saha, S. et al., (2001) Science 294, 1343-1346) and a 17-gene expression signature was recently found that is common to both primary tumours and metastases (Ramaswamy, S. et al., (2003) Nat. Genet. 33, 49-54). It was also found that a signature derived from fibroblasts which are active in wound healing can be predictive for metastasis in several tumour types (Chang, H. Y., et al. (2004) PLoS Biol. 2(2):e7, to be found at: http://www.pubmedcentral.nih.gov/articlerender.fcgi?artid=314300).

For HNSCC such expression signatures are starting to be uncovered (Chung, C. H. et al., (2004) Cancer Cell 5, 489-500), but as yet without independent validation for reliability and clinical outcome.

Thus, there is still a large need for a reliable diagnostic tool on basis of expression of genes with which a reliable and accurate prediction can be established for the presence or occurrence of lymph node metastasis in HNSCC.

### SUMMARY OF THE INVENTION

The invention now provides a nucleotide array of maximal 50 nucleotide sequences, preferably maximal 100 nucleotide sequences, more preferably maximal 1000 nucleotide sequences, for the detection of metastasis in head and neck squamous cell cancer (HNSCC) comprising at least 1 of the elements of Table 5, more preferably 2 of the elements, more preferably 3 of the elements, more preferably 4 of the elements, more preferably 5 of the elements, more preferably 6 of the elements %, more preferably 7 of the elements, more preferably 8 of the elements, more preferably 9 of the elements, more preferably 10 of the elements and most preferably at least 20 of the elements. Alternatively, a nucleotide array for the detection of metastasis in HNSCC has 50 or more of the elements of the genes listed in Table 4.

Further provided is a method to establish reference and control gene expression profiles of patients having had metastasis after HNSCC(N+ group) or no metastasis after HNSCC(N0 group) by analysing the gene expression from a tumour biopsy sample of each patient, or from pooled samples of each group of patients, on an array according to the invention.

Another embodiment of the invention is a method to predict the presence or risk of occurrence of lymph node metastasis of a HNSCC patient. comprising:

a. taking a biopsy sample from the tumour of the patient;

b. isolating the nucleic acid from the biopsy sample;

c. analyse the gene expression profile of said nucleic acid by assaying it with a nucleotide array according to the invention;

d. classifying the expression profile as N+ or N0 by determining whether the expression profile would match the expression profile of a group of HNSCC patients known to have developed metastasis.

Preferably, the biopsy samples in the above methods are fresh biopsy samples.

A preferred embodiment for the method to predict the presence or risk of metastasis is a method, wherein the analysis of the gene expression profile comprises:

a. hybridising the nucleic acid form the biopsy sample with the nucleotide array according to the invention;

b. determining the amount of hybridisation of each of the elements of the nucleotide array relative to the amount of hybridisation of each element with a reference sample, said step optionally involving a normalisation step;

c. determining for each element of the array whether the expression of the corresponding gene in the biopsy sample is more or less than the expression of the corresponding gene in the reference sample.

Preferably, the expression profile is classified as N+ (high risk of metastasis) or N0 (low or no risk of metastasis) according to the steps of:

a. determining the collective correlation of the classifier/predictor genes or elements present in the expression profile with the average N+ or N0 profile from primary tumors with previously established N-status; and

b. determining the predictive threshold based on the correlation threshold from primary tumors with previously established N-status

In another preferred embodiment the method is a method, wherein the gene expression profile of a group of HNSCC patients known to have developed metastasis is the expression profile contained in the dataset E-UMCU-11, available in the public microarray database ArrayExpress (http://www.ebi.ac.uk/arrayexpress/).

Calculation of the correlation as performed in the above methods is preferably done using the cosine correlation method.

Normalization of the expression profile is preferably achieved by correcting the expression data for experimental variations with the help of expression data of a control gene or element which is not affected by the tumour state, preferably by calculating the ratio of the expression data of each gene or element in the array of claim 1 or 2 with the expression of a control gene or element or the mean of a pool of control genes or elements.

### LEGENDS TO THE FIGURES

FIG. 1. A predictor for HNSCC lymph node metastasis. (a) Expression profiles of the 102 predictor genes on the 82 primary tumor training set (middle). The predictor genes are clustered based on their similarities across the 82 tumors (Pearson around zero correlation, centroid clustering). Tumors are rank-ordered according to their correlation with the average N0 expression profile (left). The solid line represents the threshold for optimal overall accuracy. Tumors above the threshold show an expression profile that indicates that the patient is free of lymph node metastasis. In the right panel the patient's histological N-status, including the 3 year follow-up period, and the clinical diagnosis are shown (black indicates post-operative histological N+, white indicates post-operative histological N0, dark grey indicates clinical N+ and light grey indicates clinical N0 assessment). The asterix indicates a patient that developed lymph node metastasis post-treatment. (b) Expression profiles and tumor correlations from 6 training tumors samples (circles) and their technical replicates (squares). (c) as (a) only for a independent validation set of 22 primary HNSCC tumors. The threshold is set according to the optimal threshold established with the latter half of the training set (FIG. 2d).

FIG. 2. Long-term tissue storage results in loss of predictive accuracy. (a) The mean correlation with the average no-metastasis profile and the standard deviation range for N0 patients (blue) and N+ patients (red) in the training set are shown for each year of surgery. (b) The N0 (blue), N+ (red) and overall (green) predictive accuracies increase from 40-45% for samples from 1996, to 89-100% for samples from 2000. (c, d) Correlation data from tumors with longer (c) or shorter (d) storage time. The predictor correctly predicts 22 of the 38 and 38 of the 44 samples, respectively.

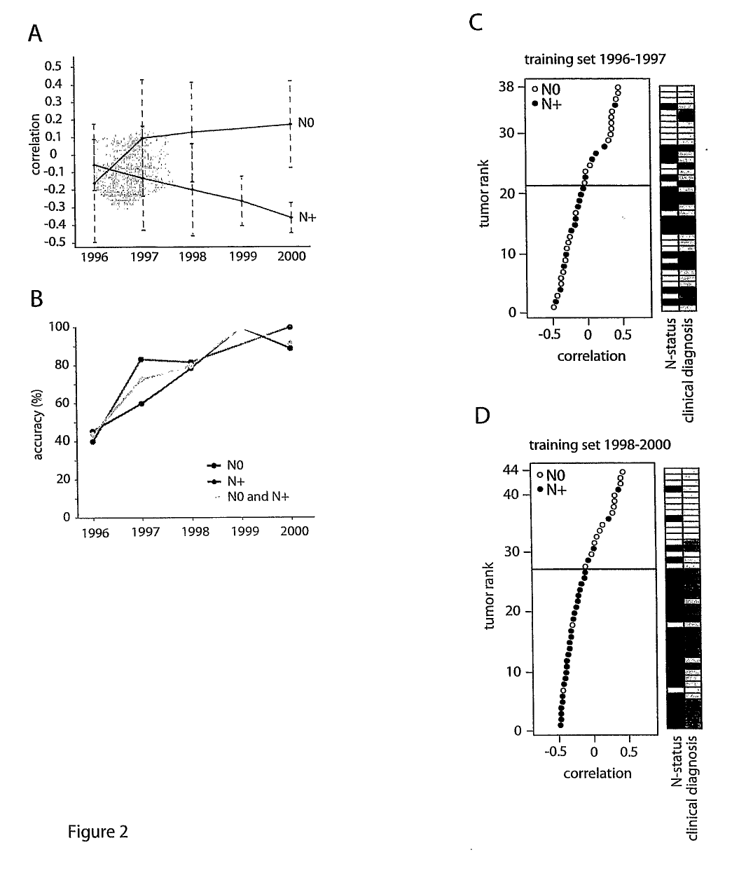

FIG. 3. The predictor outperforms current clinical diagnosis on the validation set. (a) Predictive accuracies (PA) of current clinical diagnosis (blue) and the predictor (red) on the validation set. Error bars are based on the standard error for predictive accuracy. The predictor has a N0 PA of 100%, N+ PA of 77%, and overall PA of 86%. Clinical diagnosis has a N0 PA of 67%, N+ PA of 71%, and overall PA of 68%. (b, c) Treatment accuracy for the validation set, based on current clinical diagnosis (b) or if based on predictor outcome (c). Completely appropriate treatment is shown in green and under- or overtreatment in red. Current diagnosis resulted in 23% of patients receiving appropriate treatment (50% of N+ receiving an RND). Predictor based treatment would result in 86% of patients receiving appropriate treatment (75% of N0 that no longer receive any neck dissection and 100% of N+ receiving a RND).

FIG. 4. Study design and procedures overview. a, RNA was isolated from 2-3 tumor sections, followed by mRNA amplification and fluorescent labeling. After hybridization, scanned images were quantified and the data was normalized. Duplicates of each tumor were averaged and a predictor was designed using the differentially expressed genes. Quality control monitoring occurred after total RNA isolation, cRNA synthesis, labeling, scanning and normalization. b, The training experiment design involved 82 primary HNSCC tumors, compared in duplicate dye-swap against a common reference pool containing equal amounts of cRNA from each tumor. Nine reference pool self-self comparisons were generated in parallel, to establish an error-model for technical variation. c, The predictor was designed using a double loop training-validation protocol.

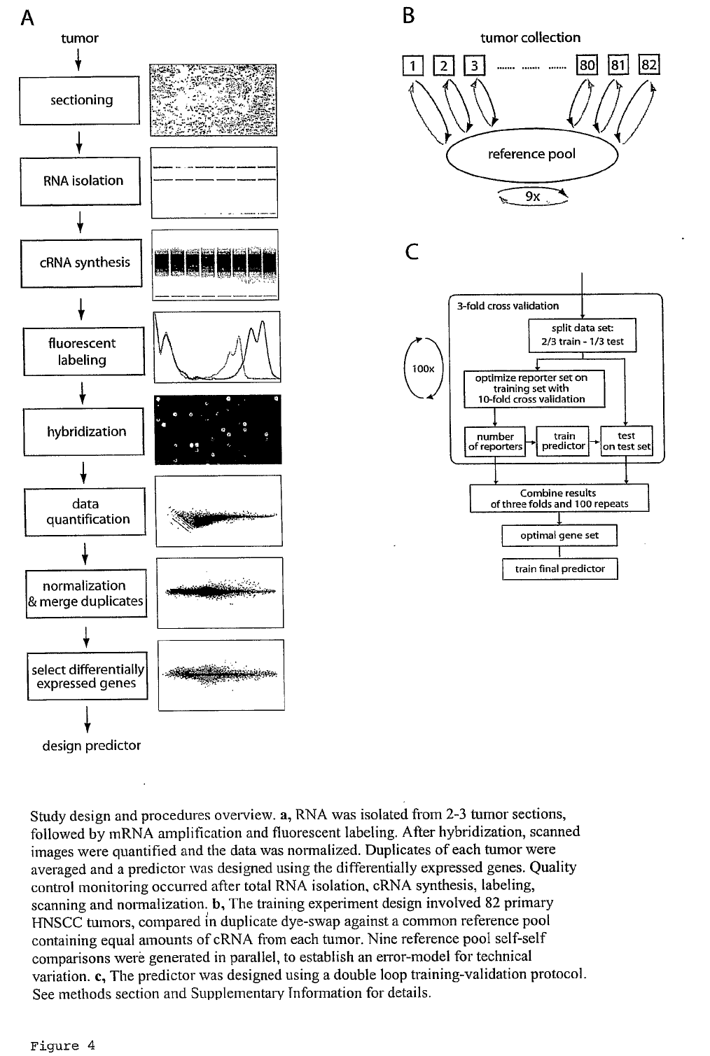

FIG. 5. The predictive outcome of different signatures is stable. Predictive correlation outcome of 66 tumor samples using a multiple training approach. A thousand different molecular signatures comprising 50 (A), 100 (B) or 200 (C) genes were used to predict each sample approximately 100 times. Samples from patients without metastasis are colored blue (top line in the graph), samples from patients with lymph node metastasis are colored red (bottom line in the graph). The shaded area represents the 95%-confidence interval for the sample predictions.

### DETAILED DESCRIPTION OF THE INVENTION

The inventors herein show that it is possible to give a more accurate prediction of the presence of lymph node metastasis of HNSCC than currently possible, by measuring mRNA expression of a concise set of genes (the predictor signature). It appeared possible to give an accurate prediction on basis of a set of 102 genes listed in Table I. It appeared that half of these genes have not been directly associated with tumorigenesis or metastasis before. Besides expected epithelial marker genes, interesting categories include genes (putatively) coding for extracellular matrix components, genes involved in cell adhesion including three members of the plakin family of cytolinkers and the enzyme transglutaminase 3, which play a role in maintaining tissue integrity; cell death genes; cell growth and maintenance genes and genes encoding hydrolyzing activities including proteins involved in degradation of the extracellular matrix (uPA and PAI-1) and a metalloproteinase. Another feature of the metastasis signature is that there is more down-regulation associated with metastasis (two thirds) than up-regulation. It is likely that this involves stromal and immune-regulatory components (Pollard, J. W. (2004) Nat. Rev. Cancer 4, 71-78; Chambers, A. F. et al. (2002) Nat. Rev. Cancer 2, 563-572). Many of the predictor genes belong to this categories, strengthening the argument for profiling bulk tumour tissue rather than laser-dissected regions densely populated with tumour cells.

It is shown herein that a diagnosis/prediction of the presence of metastases can be given using expression data of a set of only five genes from this large set of 102 genes. Table 2 indicates 15 of the genes which rank high in predictive value and which can especially be used to give a diagnosis or prediction of metastasis in HNSCC. Of course, accuracy of prediction will increase when more then five, preferably all 15 and even more preferably all 102 genes will be used on an array for gene expression analysis for the diagnostic/predictive signature.

Gene expression analysis is preferably done using a micro-array. The techniques for measuring and comparing gene expression on micro-arrays is well established within the art. It should be understood that it is not necessary to have the full length nucleotides encoding the above mentioned genes on said array: a stretch of nucleotides which is sufficient to establish unique hybridisation with the RNA expressed from said genes in the tumour cells can be used. Such a stretch of nucleotides is hereinafter referred to as ‘element’. Preferably for the specific use of gene expression analysis for the current invention (i.e. with relation to detection of the presence of or the risk for metastases of HNSCC) such an array need not contain a large number of (different) genes or elements. It would be sufficient for the array to contain the necessary genes, as discussed above, and, preferably, some control genes, as will be discussed below. The array, which can be used for the analysis of the invention thus does not need to contain more than 1000 genes or elements, preferably not more than 500 genes or elements, more preferably not more than 200 genes or elements and most preferably from about 50 to about 150 genes or elements.

To investigate a gene expression profile the array should be subjected to hybridisation with target polynucleotide molecules from a clinically relevant source, in this case e.g. a person with HNSCC. Therefore, preferably a fresh frozen (within 1 hour from surgical removal), liquid nitrogen (at least −80° C.) stored tumour sample needs to be available. Said target polynucleotide molecules should be expressed RNA or a nucleic acid derived therefrom (e.g., cDNA or amplified RNA derived from cDNA that incorporates an RNA polymerase promoter). If the target molecules consist of RNA, it may be total cellular RNA, poly(A)⁺ messenger RNA (mRNA) or fraction thereof, cytoplasmic mRNA, or RNA transcribed from cDNA (cRNA). Methods for preparing total and poly(A)⁺ messenger RNA are well known in the art, and are described e.g. in Sambrook et al., (1989) Molecular Cloning—A Laboratory Manual (2^{nd} Ed.) Vols. 1-3, Cold Spring Harbor, N.Y. In one embodiment, RNA is extracted from cells using guanidinium thiocyanate lysis followed by CsCl centrifugation (Chrigwin et al., (1979) Biochem. 18:5294-5299). In another embodiment, total RNA is extracted using a silica-gel based column, commercially available examples of which include RNeasy (Qiagen, Valencia, Calif., USA) and StrataPrep (Stratagene, La Jolla, Calif., USA). Poly(A)⁺ messenger RNA can be selected, e.g. by selection with oligo-dT cellulose or, alternatively, by oligo-dT primed reverse transcription of total cellular RNA. In another embodiment, the polynucleotide molecules analyzed by the invention comprise cDNA, or PCR products of amplified RNA or cDNA.

Preferably, the target polynucleotides are detectably labelled at one or more nucleotides. Any method known in the art may be used to detectably label the nucleotides. Preferably, this labelling incorporates the label uniformly along the length of the polynucleotide and is carried out at a high degree of efficiency. One embodiment for this labelling uses oligo-dT primed reverse transcription to incorporate the label; however, conventional methods hereof are biased toward generating 3′ end fragments. Thus, in this embodiment, random primers (e.g. 9-mers) are used in reverse transcription to uniformly incorporate labelled nucleotides over the full length of the target polynucleotides. Alternatively, random primers may be used in conjunction with PCR methods or T7 promoter-based in vitro transcription methods in order to amplify the target polynucleotides.

In a preferred embodiment, the detectable label is a luminescent label. For example, fluorescent labels, bioluminescent labels, chemiluminescent labels and calorimetric labels may be used. In a highly preferred embodiment, the label is a fluorescent label, such as a Cy5 or Cy3, fluorescein, a phosphor, a rhodamine, or a polymethine dye or derivative. In another embodiment, the detectable label is a radiolabeled nucleotide.

The array may be any nucleotide array which represents five or more of the genes of Table 2 or Table 1. To indicate the difference with the existing very large arrays of e.g. Affymetrix, the dedicated arrays of the present invention should preferably comprise no more than 50, or 100, or 250 or, alternatively 500 or 1000 genes altogether. Presence of other genes on the array is allowable and the expression data from such other genes need not necessarily be considered for the present application. The methods of the invention can be applied on the above mentioned dedicated arrays, but can also be performed on arrays that are commercially available (e.g. from Agilent US; Affymetrix Inc, CA, USA; and others). It is also possible to work with self-made arrays by spotting or synthesizing nucleotides which are known to selectively hybridise to the target genes on a surface. Methods to prepare such arrays are well within the skill of the artisan. The microarrays can comprise cDNA, but can also comprise short oligonucleotides (Affymetrix and Nimblegen) or long oligonucleotides which are synthesized in situ_(Agilent); in another embodiment the arrays comprise long oligonucleotides and are self-made by spotting.

Nucleic acid hybridisation and wash conditions are chosen so that the target polynucleotide molecules specifically hybridize to the complementary polynucleotide sequences of the array, preferably to a specific array site, wherein its complementary DNA is located. Optimal hybridisation conditions will depend on the type (e.g., RNA or DNA) of the target nucleotides and array. General parameters for specific (i.e., stringent) conditions of hybridisation are described in Sambrook et al. (supra). Typical hybridisation conditions for cDNA microarrays are hybridisation in 5×SSC plus 0.2% SDS at 65° C. four hours, followed by washes at 25° C. in low stringency wash buffer (1×SSC plus 0.2% SDS), followed by 10 minutes at 25° C. in higher stringency wash buffer (0.1×SSC plus 0.2% SDS).

When fluorescently labelled probes are used, the fluorescence emissions at each site of the microarray may be detected by scanning confocal laser microscopy. In one embodiment, the arrays is scanned with a laser fluorescent scanner with a computer controlled X-Y stage and a microscope objective. Fluorescent laser scanning devices are described in e.g. Schena et al. (1996) Genome Res. 6:639-645. Signals are recorded and, in a preferred embodiment, analysed by computer using a 12 or 16 bit analog to digital board. In one embodiment the scanned image is despeckled using a graphics program (e.g., Hijaak Graphics Suite) and then analysed using an image gridding program that creates a spreadsheet of the average hybridisation at each wavelength at each site.

Not all of the genes are evenly contributing to the discriminating effect. As is shown in Table 1, the genes differ in significant expression. Although the statistical data presented in the Examples are calculated with all of the 102 genetic elements of Table 1, it is submitted that a good distinction between the two groups of patients and therewith a good diagnosing/predicting ability of the signature gene set can also be achieved with only a part of the elements of Table 1. At least 5 (5%) of the elements of Table 1 are included in the analysis, more preferably 20%, more preferably 40%, more preferably 60%, more preferably 80%, more preferably 90% and most preferably all of the elements. It would be advisable not to randomly choose the elements, but to pick the most discriminating genes in this list. Table 2 gives an overview of the top 15 genes out of the 102 genes of table 1, of which at least 5, more preferably at least 6, more preferably at least 7, more preferably at least 8, more preferably at least 9, more preferably at least 10, more preferably at least 11, more preferably at least 12, more preferably at least 13, more preferably at least 14, and most preferably all 15 can be used for making up the signature with which the microarray analysis is performed.

It furthermore has been found that a more comprehensive set of predicting genes can be compiled by repeatedly calculating a predictive signature via a multiple training approach (similar to Michiels, S. et al., Lancet 365:488-492, 2005). In this study (see Examples) it appeared that from the originally more than 2000 differentially expressed genes only 825 (Table 3) had a predictive character, and that for these a subgroup of 179 (Table 4) genes was used in more than half of the signatures. From this group again a supergroup of 61 genes (Table 5) could be distinguished which was predominantly used to discriminate between N+ and N0. It will be understood that preferably an array would comprise at least three, but preferably five, more preferably 10, even more preferably 25 and most preferably 61 of the genes of Table 65. However, it also appeared possible to classify on basis of genes, which did not occur in Table 5, but in such cases many genes are required to achieve an acceptable prediction. Thus, an array could also comprise at least 10, preferably 25, more preferably 50, and most preferably 100 of the genes of Table 5.

As indicated above, various combinations of these genes can be used for determining the presence of lymph node metastases in several ways.

On dual channel DNA microarrays this is performed by determining the expression level ratios of the genes in the primary tumour sample versus expression of the same genes in reference material. The reference material can be derived from a pool of total RNA or amplified mRNA from a set of HNSCC primary tumours with established lymph node metastasis characteristics. The individual gene expression ratios contribute towards the expression ratio signature of a sample. The degree of correlation of a sample's signature with the signatures of samples with known metastatic status (preferably calculated by the cosine correlation (Jones, W. P., & Furnas, G. W. (1987). Pictures of Relevance: A Geometric Analysis of Similarity Measures. Journal of the American Society for Information Science, 36 (6), 420-442) as, e.g, provided by the Genesis software; http://genome.tugraz.at/Software/Genesis/Description.html) is used to predict the metastatic state of the unknown sample. The correlation threshold for predicting the metastatic state is based on the optimal threshold for discriminating between the metastatic states of the samples with known metastatic states, which can easily be determined by a person skilled in the art

Other measurements of absolute expression and expression ratios of these genes can also be used. Reference material can be derived from other sources than a pool of samples with known metastatic states. Preferably, however, samples with known metastatic states are still required to determine the correlation threshold for determining the metastatic status.

Expression ratios can also be derived from single channel microarray experiments, using as a reference so-called housekeeping genes (i.e. with stable expression across many different samples) or a collection of housekeeping genes or any collection of genes or features with stable expression. Again here it is preferred to use samples with known metastatic states to determine the correlation threshold for determining the metastatic status.

Gene expression measurements and the derived ratios can also be obtained by (quantitative) reverse transcription PCR or any other assay for gene expression, using as a reference any gene or collection of genes that have stable expression across many samples. In a specific embodiment of this application of the invention, samples with known metastatic states are still required to determine the correlation threshold for determining the metastatic status.

In the absence of tumour samples with known metastatic states for calibration of the prediction, the genes or various combinations of the (expression analysis of the) genes can still be used to predict the metastatic state. In these embodiments of the invention an absolute or relative measurement of gene expression is determined for example using single or dual channel DNA microarrays, or by other methods such as (quantitative) reverse transcription PCR. Increased expression of the genes in table 1 or 2 with a positive N+ correlation will hereby contribute positively towards prediction of the N+ status and negatively towards prediction of the N0 status. Conversely, increased expression of the genes in table 1 or 2 with a negative N+ correlation will contribute positively towards N0 prediction and negatively towards N+ prediction. Increased expression in both cases indicates an increase relative to a suitable marker gene or feature, set of genes or features or collectively in relation to each other.

However, a person skilled in the art is able to obtain the reference data that have been produced in the below example, since this data is available as dataset E-UMCU-11 from the public micro-array database ArrayEcpress (http://www.ebi.ac.uk/arrayexpress/). When desiring to predict or determine the presence of metastases for a certain patient, the practitioner should take a biopsy from that patient, isolate the RNA and determine the expression of at least 5 of the elements of Table 1. To normalize these expression data with respect to the data of the reference set E-UMCU-11, it is possible to correct the data for variations with the help of expression data of a control gene or element which is not affected by the tumour state (such as a housekeeping gene), which is present in the reference set E-UMCU-11 and should also be available on the array that has been used to determine the expression profile of the patient to be assessed. In stead of one control gene or element, also the mean value of a poll of control genes or elements can be taken. This correction can, for instance, be done by subtracting the expression level of the control gene(s)/element(s) from the expression levels of each of the tested genes/elements. Preferably, the ratio for every tested gened with respect to the control gene(s) is calculated for both the patient's expression profile as well as for the expression data of the reference set.

With these figures, the correlation with the mean value of the N0 values of the reference set should be calculated. If this correlation is negative (i.e. a value below zero) it can be concluded that the patient is N+ (i.e. having or prone to develop metastases). Conversely, the correlation can be calculated with respect to the mean value of the N+ values of the reference set. Then a negative correlation indicates a match with the N0 group.

Further enablement for a diagnosis/prediction of cancer metastasis on basis of gene expression analyses can be found in WO 03/010337, indicating that methods as have been generally described above are well within the skills of the practitioners in the art.

### EXAMPLE

### Data Accessibility

MIAME¹ compliant data in MAGE-ML² format as well as complete descriptions of protocols, microarrays and clinical parameters have been submitted to the public microarray database ArrayExpress (http://www.ebi.ac.uk/arrayexpress/) with the following accession numbers: Microarray layout, A-UMCU-3; HNSCC tumour data, E-UMCU-11; Protocols for sectioning of tumour material, P-UMCU-18; RNA isolation, P-UMCU-19; DNase treatment, P-UMCU-20; mRNA amplification, P-UMCU-21; generating reference pool, P-UMCU-26; cRNA labeling, P-UMCU-22; hybridization and washing of slides, P-UMCU-23 and P-UMCU-24; scanning of slides, P-UMCU-25; Image analysis, P-UMCU-11

### Tumor Samples

For the training set, 92 samples were randomly taken from a collection of primary tumours surgically removed between 1996 and 2000 and that fulfilled the following criteria: biopsy-proven HNSCC in the oropharynx and oral cavity; no previous malignancies in the head and neck region; tumour sections contained more than 50% tumour cells. Of these 92 tumours, 82 passed total RNA and cRNA quality control (QC) and were included in this study. For the validation set, 27 tumours were randomly taken from the same collection of tumours, surgically removed between 2000 and March 2001, and that fulfilled the same selection criteria. Of these, 22 passed total RNA and cRNA QC and were included in this study. The diagnostic procedures for clinical staging of cervical lymph nodes was performed according to the Netherlands national guidelines for oral cavity and oropharynx carcinomas, by clinical examination (palpation) of the neck region, followed by bilateral ultrasound examination, computed tomography (CT) and/or magnetic resonance imaging (MRI). Suspected nodes were subjected to aspiration cytology. In this way, patients were pre-operatively classified as either N0 or N+, the latter in the case of aspirates yielding metastatic tumour cells. Only in the case of obvious neck involvement, as shown by huge swelling, were the patients classified as N+ without additional efforts to prove the presence of metastasis.

Surgery was aimed at complete tumour removal. With regard to the neck, in the case of clinical N0 only a SOHND was performed³. In cases clinically classified as N+ a RND was performed including all five lymph node levels³. Postoperative irradiation was administered in accordance with current practice and depending on margin status, tumour growth features, number of positive nodes and extracapsular growth. In practice, 36 out of 60 clinically assessed N0 patients and 38 out of 43 clinically assessed N+ patients received radiation therapy. This treatment as well as additional clinical information is presented in Supplemental data 2 (for accessibility, see above). After surgery, patients were periodically checked for development of neck metastasis, and patients initially classified as N0 but showing positive nodes in their surgical specimen or developing neck nodes within a time span of 3 years after surgery without having another head and neck cancer that could be responsible for this metastasis, were retrospectively added to the N+ patient group. Less than 5% of patients with HNSCC in the oral cavity or oropharynx subsequently develop metastasis after treatment^{4,5}. Here, for the training and validation cohorts, one patient subsequently developed positive neck nodes after surgery. Three years is to be considered as a reliable time period, since at least 80% of the recurrences are known to take place in the first two years after surgery (Takes, R. P. et al. (2001) J Pathol 194, 298-302; Jones, K. R., et al., (1992) Arch. Otolaryngol. Head Neck Surg. 118, 483-485)

Fresh tumour tissue was taken from the surgical specimen, snap-frozen in liquid nitrogen immediately after surgical removal and stored at −80° C. Frozen sections were cut for RNA isolation and immediately transferred to a RNAlater solution (Ambion). A haematoxylin and eosin stained section was prepared for tumour percentage assessment. Only samples with at least 50 percent tumour cells were used. For a small number of samples the tumour percentage was increased by removing areas with no tumour cells.

### RNA Isolation

Total RNA was isolated from 2-3 sections (20 μm) with TRIzol reagent (Invitrogen), followed by a purification using the RNeasy mini-kit (Qiagen) and a DNase treatment using the Qiagen DNA-free kit. The yield and quality of total RNA was checked by spectrophotometry and by the Agilent 2100 Bioanalyser (Agilent). Total RNA quality control criteria were in accordance with the Tumour Analysis Best Practices Working Group⁶, discarding samples with no clear 18S and 28S ribosomal bands. We also removed samples that had a yield lower than 500 ng total RNA or showed mycoplasma contamination.

cRNA Synthesis and Labeling

mRNA was amplified by in vitro transcription using T7 RNA polymerase on 1 μg of total RNA. First a double stranded cDNA template was generated including the T7 promoter. Next, this template was used for in vitro transcription with the T7 megascript kit (Ambion) to generate CRNA. During the in vitro transcription, 5-(3-aminoallyl)-UTP (Ambion) was incorporated into the single-stranded cRNA. The yield and quality of the cRNA was analyzed by spectrophotometry and by the Agilent 2100 Bioanalyser. Samples with a yield less than 5000 ng or with small cRNA fragments (median less than 500 bp) were not used.

Cy3 or cy5 fluorophores (Amersham) were coupled to 500 ng of cRNA. After coupling, free dye molecules were removed using Clontech ChromoSpin-30 columns (Clontech). The yield and label incorporation (5-7%) of the cy-labeled cRNA was checked using spectrophotometry. Before hybridization, 300 ng of cy-labeled cRNA from one tumor was mixed with an equal amount of reverse color cy-labeled material from the reference sample.

### Microarray Production

The Human Array-Ready Oligo set (version 2.0) was purchased from Qiagen and printed on Corning UltraGAPS slides as described elsewhere⁷. The microarrays contained 70-mer oligonucleotides representing 21,329 genes as well as 3871 additional features for control purposes.

### Microarray Hybridization

Before use, the microarray slides were treated with sodium-borohydrate solution to reduce auto-fluorescence in the cy3-channel⁸. The labelled cRNA targets were hybridized on the microarray for 10 hours at 42° C. using the Ventana Discovery Hybridization Station in combination with the ChipMap-80 Kit (Ventana Europe). After hybridization the slides were manually washed and scanned in the Agilent G2565AA DNA Microarray Scanner (100% laser power, 30% PMT).

### Pre-Processing of Expression Data

The scanned images were quantified and background corrected using Imagene 4.0 software (Biodiscovery). The expression data was normalized for dye and print-tip biases using a Lowess per print-tip normalization algorithms applied in the statistical package R¹⁰. Following normalization, variance stabilization (VSN)¹¹ was applied to stabilize variance in the intensity data. Both duplicate dye-swap hybridizations of each tumour were averaged and for each gene a tumour-reference ratio was calculated. Reference versus reference hybridizations were used to build a gene error model for technical variation. Nine reference self-self comparisons were performed in dye-swap (18 hybridizations), resulting in nine reference ratios for each gene on the microarray. These nine reference ratios yield an estimate of the technical variation for each gene. To test whether a gene in a tumour samples shows differential expression, a Student's t-test was applied on the tumour ratio and the corresponding nine reference ratios (technical variation). The calculated p-values for differential expression were used to select those genes that show differential expression in the tumour samples.

### Supervised Classification

A classifier was constructed to distinguish between N0 and N+ patients. Of the 21,329 genes on the microarray, 6221 were excluded based on aberrant signal and spot morphology in one of the 164 hybridizations. From these remaining 15,108 genes, only genes that were significantly different from the reference in at least 31 tumours were selected based on the error model for technical variation (p<0.01). This resulted in a set of 1,986 genes. For these genes the signal-to-noise-ratio (SNR)¹² was computed and employed to rank the genes (top ranked genes being genes that are best suited to distinguish the outcome classes). The optimal gene set to employ in the classifier (a nearest mean classifier similar to the classifier employed in¹³), was determined by gradually expanding the gene set starting from the highest ranked gene. At each expansion round the nearest mean classifiers was trained on a training set and tested on a test set. The performance on the test set served as a quality measure of the gene set. The performance was measured as the average of the false positive (N0 classified as N+) and false negative (N+ classified as N0) rates of the test samples. Initially the performance increases as the set is expanded. The expansion of the gene set is terminated when the performance deteriorates, i.e. when the optimal performance is reached. The steps of ranking the genes and training and testing the classifier are performed in a 10-fold cross-validation procedure. The output of this procedure is an optimal number of top-ranked genes and a trained classifier. To ensure independent validation, this process of optimizing the set of genes and training the classifier is wrapped in a second 3-fold cross-validation loop. This entails that the optimization of the gene set and the training of the classifier is performed on ⅔ of the data, while the classifier is validated on ⅓ of the data. Since this ⅓ of the data is never involved in any of the gene selection and training steps, this ensures completely independent validation of the classifier, which avoids selection bias^{14,15} and therefore results in a reliable performance estimate. This double-loop procedure determined 102 genes to form the final diagnostic classifier. This classifier was trained on the complete set of 82 samples by recalculating the signal-to-noise ratio for all genes and subsequently selecting the top 102 genes. The predictor was trained using the 102 selected genes and the 82 training samples. A decision threshold for this classifier was fixed such that the highest overall predictive accuracy for both N0 and N+ tumours. was reached.

### Statistics

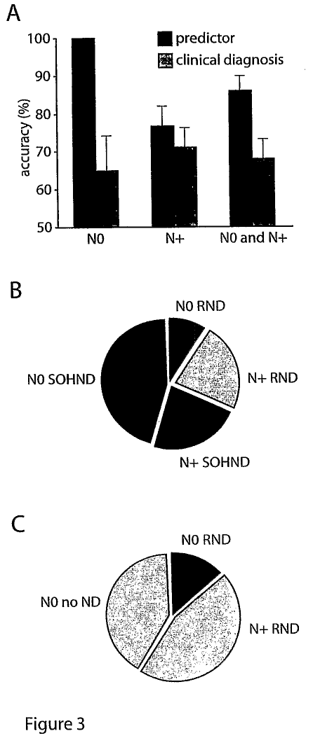

Odds ratios (OR) were calculated by fitting a logistic regression model on the prediction outcome of the validation set. The predictor had an infinitive OR since no false negative prediction was made. To get an estimate of the OR for the predictor, one false negative was artificially introduced resulting in a predictor OR of 30 (p=0.006) and a clinical OR of 4.2 (p=0.15). The standard error for predictive accuracy (FIG. 3a) includes the predictions made on the latter half of the training set.

### Selection of Predictive Genes

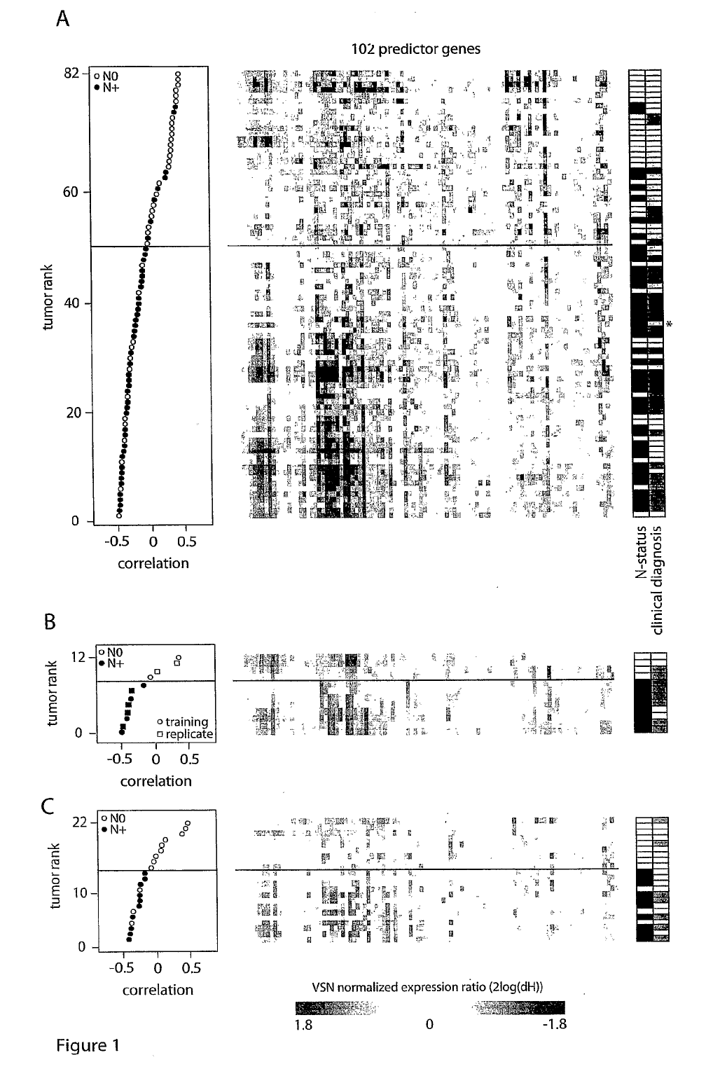

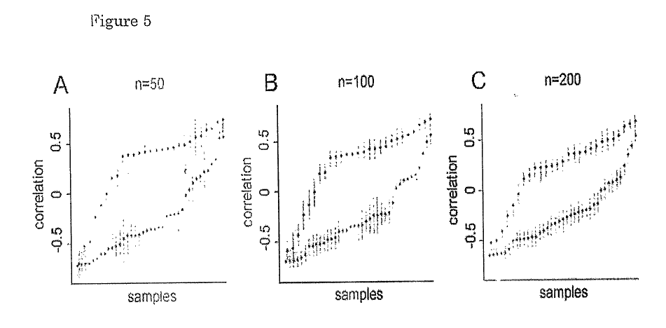

A multiple training approach was used to identify a complete set of predictive genes, based on the 66 tumor samples from 1998 to 2001. The tumor samples were randomly divided into a training set and test set using a 10-fold cross validation procedure. Based on the training set, P-values were calculated for all 3064 differentially expressed genes based on the difference in expression between N+ and N0 tumor samples (Student's T-test). The set of genes with lowest P-values (i.e. most-predictive) was used for prediction of the test samples by calculating the correlation with the average N+ and average N0 training profile and, based on these correlations, classifying the test samples as N0 or N+. Repeating this resampling procedure a thousand times resulted in multiple predictions for each tumor sample, based on the different predictive gene sets. This approach was repeated three times to determine 1000 predictive gene sets consisting of 50 genes, 1000 gene sets of 100 genes and 1000 gene sets of 200 genes. All gene sets had predictive value (FIG. 1). Genes selected at least once are listed in Table 3. This consists of 825 genes with predictive power for detection or prediction of metastasis in head and neck squamous cell carcinoma. Small and large sets of genes from this list can be used for prediction (FIG. 5). Genes selected more frequently, that is present in more than 50% of the 200 gene set predictors are listed in Table 4. This consists of 179 genes with strongest predictive power for detection or prediction of metastasis in head and neck squamous cell carcinoma. Small and large sets of genes from this list can be used for prediction. Genes selected most frequently (more than 90%) are listed in Table 5. This consists of 51 genes with the highest predictive power. Small and large sets of genes from this list can be used for prediction. This list consists of genes, most/all of which have never before been associated with prediction of metastasis in tumors, especially metastasis in head-neck squamous cell carcinoma.

### REFERENCES

1. Brazma, A. et al. Minimum information about a microarray experiment (MIAME)-toward standards for microarray data. Nat. Genet. 29, 365-371 (2001).
2. Spellman, P. T. et al. Design and implementation of microarray gene expression markup language (MAGE-ML). Genome Biol. 3, RESEARCH0046 (2002).
3. Robbins, K. T. et al. Neck dissection classification update: revisions proposed by the American Head and Neck Society and the American Academy of Otolaryngology-Head and Neck Surgery. Arch. Otolaryngol. Head Neck Surg. 128, 751-758 (2002).
4. Carvalho, A. L., Kowalski, L. P., Borges, J. A., Aguiar, S., Jr. & Magrin, J. Ipsilateral neck cancer recurrences after elective supraomohyoid neck dissection. Arch. Otolaryngol. Head Neck Surg. 126, 410-412 (2000).
5. Ridge, J. A. Squamous cancer of the head and neck: surgical treatment of local and regional recurrence. Semin. Oncol. 20, 419-429 (1993).
6. Expression profiling-best practices for data generation and interpretation in clinical trials. Nat. Rev. Genet. 5, 229-237 (2004).
7. van de Peppel, J. et al. Monitoring global messenger RNA changes in externally controlled microarray experiments. EMBO Rep. 4, 387-393 (2003).
8. Raghavachari, N., Bao, Y. P., Li, G., Xie, X. & Muller, U. R. Reduction of autofluorescence on DNA microarrays and slide surfaces by treatment with sodium borohydride. Anal. Biochem. 312, 101-105 (2003).
9. Yang, Y. H. et al. Normalization for cDNA microarray data: a robust composite method addressing single and multiple slide systematic variation. Nucleic Acids Res. 30, e15 (2002).
10. Ihaka, R. & Gentleman, R. R: a language for data analysis and graphics. J. Comp. Graph. Statist. 5, 299-314 (1996).
11. Huber, W., von Heydebreck, A., Sultmann, H., Poustka, A. & Vingron, M. Variance stabilization applied to microarray data calibration and to the quantification of differential expression. Bioinformatics 18 Suppl 1, S96-S104 (2002).
12. Golub, T. R. et al. Molecular classification of cancer: class discovery and class prediction by gene expression monitoring. Science 286, 531-537 (1999).
13. van 't Veer, L. J. et al. Gene expression profiling predicts clinical outcome of breast cancer. Nature 415, 530-536 (2002).
14. Simon, R., Radmacher, M. D., Dobbin, K. & McShane, L. M. Pitfalls in the use of DNA microarray data for diagnostic and prognostic classification. J. Natl. Cancer Inst. 95, 14-18 (2003).
15. Ambroise, C. & McLachlan, G. J. Selection bias in gene extraction on the basis of microarray gene-expression data. Proc Natl Acad Sci USA 99, 6562-6566 (2002).

TABLE 1
Complete list of the 102 HNSCC predictor genes

| Gene | GenBank ID | Gene name | N+ corr |
|---|---|---|---|
| FTH1 | NM_002032 | ferritin, heavy polypeptide | 0.726 |
| | | 1 | |
| COL5A1 | NM_000093 | COL5A1 protein (collagen | 0.627 |
| | | 5 type A1 like) | |
| NCOR2 | NM_006312 | Nuclear receptor co- | 0.617 |
| | | repressor 2 | |
| P4HA1 | NM_000917 | proline 4-hydroxylase | 0.572 |
| | | alpha polypeptide I | |
| TNFAIP3 | NM_006290 | tumour necrosis factor, | 0.536 |
| | | alpha-induced protein 3 | |
| PLAU | NM_002658 | urokinase plasminogen | 0.535 |
| | | activator (uPA) | |
| COL5A3 | NM_015719 | Collagen, type V, alpha 3 | 0.521 |
| SPOCK | NM_004598 | Sparc/osteonectin, cwcv | 0.500 |
| | | and kazal-like domains | |
| | | proteoglycan (testican) | |
| FAP | NM_004460 | fibroblast activation | 0.498 |
| | | protein, alpha | |
| ADAM12 | NM_003474 | a disintegrin and | 0.482 |
| | | metalloproteinase domain | |
| | | 12 (meltrin alpha) | |
| TPM2 | NM_003289 | tropomyosin 2 (beta) | 0.470 |
| MICAL2 | NM_014632 | flavoprotein | 0.459 |
| | | oxidoreductase MICAL2 | |
| D2S448 | XM_056455 | D2S448 (Melanoma | 0.446 |
| | | associated gene) | |
| PAI-1 | NM_000602 | plasminogen activator | 0.440 |
| | | inhibitor type 1 | |
| REN | NM_000537 | renin | 0.383 |
| POSTN | NM_006475 | periostin, osteoblast | 0.367 |
| | | specific factor | |
| CKTSF1B1 | NM_013372 | cysteine knot superfamily | 0.330 |
| | | 1, BMP antagonist 1 | |
| | | (DRM/GREMLIN) | |
| IER3 | NM_003897 | immediate early response | 0.303 |
| | | 3 | |
| MMD | NM_012329 | monocyte to macrophage | 0.300 |
| | | differentiation-associated | |
| CTDSP1 | NM_021198 | CTD (carboxy-terminal | 0.260 |
| | | domain, RNA polymerase | |
| | | II, polypeptide A) small | |
| | | phosphatase 1 | |
| PSMD2 | NM_002808 | proteasome (prosome, | 0.256 |
| | | macropain) 26S subunit, | |
| | | non-ATPase, 2 | |
| MAN1B1 | NM_016219 | mannosidase, alpha, class | 0.254 |
| | | 1B, member 1 | |
| DKK3 | NM_013253 | dickkopf homolog 3 | 0.140 |
| | | (Xenopus laevis) | |
| NT5C3 | NM_016489 | 5′-nucleotidase, cytosolic | 0.138 |
| | | III | |
| DAPK3 | NM_001348 | death-associated protein | 0.022 |
| | | kinase 3 | |
| NDUFB4 | NM_004547 | NADH dehydrogenase | 0.012 |
| | | (ubiquinone) 1 beta | |
| | | subcomplex, 4, 15 kDa | |
| UBA52 | NM_003333 | ubiquitin A-52 residue | −0.002 |
| | | ribosomal protein fusion | |
| | | product 1 | |
| C9orf5 | NM_032012 | chromosome 9 open | −0.075 |
| | | reading frame 5 | |
| COPG | NM_016128 | Coat protein gamma-cop | −0.081 |
| RGS5 | NM_003617 | regulator of G-protein | −0.097 |
| | | signalling 5 | |
| FLJ12236 | AK022298 | Homo sapiens cDNA | −0.104 |
| | | FLJ12236 fis, clone | |
| | | MAMMA1001244 | |
| IDI1 | NM_004508 | Isopentenyl-diphosphate | −0.126 |
| | | delta isomerase | |
| RPL37A | NM_000998 | ribosomal protein L37a | −0.162 |
| ZDHHC18 | NM_032283 | Zinc finger DHHC domain | −0.175 |
| | | containing protein 18 | |
| MO30 | M26463 | Homo sapiens | −0.194 |
| | | immunoglobulin mu chain | |
| | | antibody MO30 (IgM) | |
| | | mRNA, complete cds | |
| FLJ20073 | NM_017654 | Hypothetical protein | −0.215 |
| | | FLJ20073 | |
| FLJ30814 | AK055376 | Homo sapiens cDNA | −0.222 |
| | | FLJ30814 fis, clone | |
| | | FEBRA2001529 | |
| PLK2 | NM_006622 | polo-like kinase 2 | −0.230 |
| MMPL1 | NM_004142 | Matrix metalloproteinase- | −0.230 |
| | | like 1 | |
| | Z95126 | Human DNA sequence | −0.232 |
| | | from clone RP1-30P20 on | |
| | | chromosome Xq21.1-21.3 | |
| BAL | NM_031458 | B aggressive lymphoma | −0.234 |
| | | protein (BAL) | |
| OSBP2 | NM_030758 | Oxysterol binding protein | −0.244 |
| | | 2 | |
| PARVB | NM_013327 | Parvin, beta | −0.286 |
| CEBPA | NM_004364 | CCAAT/enhancer binding | −0.290 |
| | | protein (C/EBP), alpha | |
| ZNF533 | NM_152520 | zinc finger protein 533 | −0.291 |
| ABCA12 | NM_015657 | ATP-binding cassette, sub- | −0.293 |
| | | family A (ABC1), member | |
| | | 12 | |
| LOR | NM_000427 | loricrin | −0.322 |
| E2F5 | NM_001951 | E2F transcription factor 5, | −0.356 |
| | | p130-binding | |
| APM2 | NM_006829 | Adipose specific 2 | −0.360 |
| CAPNS2 | NM_032330 | CAPNS2 (calpain small | −0.360 |
| | | subunit 2) | |
| MGC13219 | NM_032931 | Hypothetical protein | −0.367 |
| | | MGC13219 | |
| FLJ22202 | NM_024883 | Hypothetical protein | −0.383 |
| | | FLJ22202 | |
| KRT23 | NM_173213 | keratin 23 (histone | −0.409 |
| | | deacetylase inducible) | |
| PPT2 | NM_005155 | palmitoyl-protein | −0.417 |
| | | thioesterase 2 | |
| PGBD5 | NM_024554 | piggyBac transposable | −0.469 |
| | | element derived 5 | |
| SSH2 | NM_033389 | SSH2 (slingshot 2) | −0.475 |
| ALOX12B | NM_001139 | arachidonate 12- | −0.480 |
| | | lipoxygenase, 12R type | |
| MAL2 | NM_052886 | mal, T-cell differentiation | −0.544 |
| | | protein 2 | |
| ZD52F1 | NM_033317 | Hypothetical gene ZD52F1 | −0.562 |
| EPPK1 | AB107036 | Epiplakin 1 | −0.566 |
| S100A7 | NM_002963 | S100 calcium binding | −0.580 |
| | | protein A7 (psoriasin 1) | |
| FLJ22184 | NM_025094 | Hypothetical protein | −0.586 |
| | | FLJ22184 | |
| MAP17 | NM_005764 | membrane-associated | −0.591 |
| | | protein 17 (MAP17) | |
| FLJ13497 | AK023559 | Homo sapiens cDNA | −0.603 |
| | | FLJ13497 fis, clone | |
| | | PLACE14518 | |
| ECM1 | NM_022664 | extracellular matrix | −0.610 |
| | | protein 1 | |
| TGM3 | NM_003245 | transglutaminase 3 (E | −0.629 |
| | | polypeptide, protein- | |
| | | glutamine-gamma- | |
| | | glutamyltransferase) | |
| RAD17 | NM_133344 | RAD 17 homolog (S. | −0.631 |
| | | pombe) | |
| FLJ30988 | AK055550 | Homo sapiens cDNA | −0.650 |
| | | FLJ30988 fis, clone | |
| | | HLUNG1000030 | |
| C10orf26 | NM_017787 | chromosome 10 open | −0.653 |
| | | reading frame 26 | |
| PALM2 | NM_053016 | paralemmin 2 | −0.655 |
| C4.4A | NM_014400 | GPI-anchored metastasis- | −0.665 |
| | | associated protein homolog | |
| | | (C4.4A) | |
| ECG2 | NM_032566 | Esophagus cancer-related | −0.670 |
| | | gene-2 protein precursor | |
| | | (ECRG-2) | |
| PPL | NM_002705 | periplakin | −0.672 |
| HPCAL1 | NM_002149 | hippocalcin-like 1 | −0.674 |
| SLPI | NM_003064 | secretory leukocyte | −0.677 |
| | | protease inhibitor | |
| | | (antileukoproteinase) | |
| PI3 | NM_002638 | protease inhibitor 3, skin- | −0.680 |
| | | derived (SKALP) | |
| FLJ25911 | AK098777 | Hypothetical protein | −0.680 |
| | | FLJ25911 [Fragment] | |
| CLIC3 | NM_004669 | chloride intracellular | −0.691 |
| | | channel 3 | |
| BENE | NM_005434 | BENE protein | −0.698 |
| FLJ00074 | AK024480 | FLJ00074 protein | −0.699 |
| | | [Fragment] | |
| DKFZp547F | AL512697 | Homo sapiens mRNA; | −0.706 |
| 134 | | cDNA DKFZp547F134 | |
| | | (from clone | |
| | | DKFZp547F134) | |
| PLA2G4B | NM_005090 | phospholipase A2, group | −0.707 |
| | | IVB (cytosolic) | |
| | AF339799 | Homo sapiens clone | −0.708 |
| | | IMAGE: 2363394, mRNA | |
| | | sequence | |
| TRGV9 | BC062761 | T cell receptor gamma | −0.710 |
| | | variable 9 | |
| DSG3 | NM_001944 | desmoglein 3 (pemphigus | −0.711 |
| | | vulgaris antigen) | |
| FLJ12787 | NM_032175 | Hypothetical protein | −0.734 |
| | | FLJ12787 | |
| LLGL2 | NM_004524 | lethal giant larvae | −0.738 |
| | | homolog 2 (Drosophila) | |
| SMC5L1 | NM_015110 | SMC5 structural | −0.741 |
| | | maintenance of | |
| | | chromosomes 5-like 1 | |
| ODCP | NM_052998 | Ornithine decarboxylase- | −0.741 |
| | | like protein (EC 4.1.1.17) | |
| | | (ODC-paralogue) (ODC-p) | |
| FLJ31161 | AK055723 | Homo sapiens cDNA | −0.747 |
| | | FLJ31161 fis, clone | |
| | | KIDNE1000028 | |
| FLJ21214 | AK024867 | Homo sapiens cDNA: | −0.748 |
| | | FLJ21214 fis, clone | |
| | | COL00523 | |
| SPINK5 | NM_006846 | serine protease inhibitor, | −0.749 |
| | | Kazal type, 5 | |
| KIAA0350 | XM_290667 | KIAA0350 protein | −0.760 |
| PGLYRPL | NM_052890 | peptidoglycan recognition | −0.764 |
| | | protein L precursor | |
| S100A9 | NM_002965 | S100 calcium binding | −0.773 |
| | | protein A9 (calgranulin B) | |
| DNAH11 | NM_003777 | Dynein, axonemal, heavy | −0.776 |
| | | chain-11 | |
| LAGY | NM_032495 | lung cancer-associated Y | −0.793 |
| | | protein; homeodomain- | |
| | | only protein | |
| IVL | NM_005547 | involucrin | −0.801 |
| TNFRSF5 | NM_001250 | tumor necrosis factor | −0.802 |
| | | receptor superfamily, | |
| | | member 5 (CD40) | |
| SRP19 | NM_003135 | signal recognition particle | −0.814 |
| | | 19 kDa | |
| KLK12 | NM_019598 | kallikrein 12 | −0.837 |
| IL22RA1 | NM_021258 | interleukin 22 receptor, | −0.885 |
| | | alpha 1 | |

TABLE 2

| Gene | GenBank ID | Gene name | N+ corr. |
|---|---|---|---|
| COL5A3 | NM_015719 | Collagen, type V, alpha 3 | 0.520719 |
| COL5A1 | NM_000093 | COL5A1 protein (collagen 5 | 0.626949 |
| | | type A1 like) | |
| ZD52F1 | NM_033317 | Hypothetical gene ZD52F1 | −0.56185 |
| FLJ25911 | AK098777 | Hypothetical protein FLJ25911 | −0.6799 |
| | | [Fragment] | |
| EPPK1 | AB107036 | Epiplakin 1 | −6.56641 |
| KIAA0350 | XM_290667 | KIAA0350 protein | −0.76021 |
| DNAH11 | NM_003777 | Dynein, axonemal, heavy | −0.77599 |
| | | chain-11 | |
| PI3 | NM_002638 | protease inhibitor 3, skin- | −0.67983 |
| | | derived (SKALP) | |
| P4HA1 | NM_000917 | procollagen-proline, | 0.572479 |
| | | 2-oxoglutarate 4-dioxygenase | |
| | | (proline 4-hydroxylase), | |
| | | alpha polypeptide I | |
| ODCP | NM_052998 | Ornithine decarboxylase-like | −0.74146 |
| | | protein (EC 4.1.1.17) (ODC- | |
| | | paralogue) (ODC-p) | |
| MMD | NM_012329 | monocyte to macrophage | 0.299881 |
| | | differentiation-associated | |
| TPM2 | NM_003289 | tropomyosin 2 (beta) | 0.470038 |
| SRP19 | NM_003135 | signal recognition particle | −0.81361 |
| | | 19 kDa | |
| IL22RA1 | NM_021258 | interleukin 22 receptor, | −0.88482 |
| | | alpha 1 | |
| FLJ31161 | AK055723 | Homo sapiens cDNA FLJ31161 | −0.7475 |
| | | fis, clone KIDNE1000028 | |

TABLE 3
List of 825 genes with predictive value

| | | | | N+ | |
|---|---|---|---|---|---|
| UniProt | Gene_symbol | GB_accession | UniGene_ID | correlation | Gene |
| Q9NZQ6 | COL5A3 | NM_015719 | 235368 | 0.57481098 | Collagen, type V, alpha 3 |
| FINC_HUMAN | FN1 | NM_002026 | 287820 | 0.55126846 | Fibronectin 1 |
| FSL1_HUMAN | FSTL1 | NM_007085 | 296267 | 0.53452734 | Follistatin-like 1 |
| ER22_HUMAN | KDELR2 | NM_006854 | 118778 | 0.52986731 | KDEL (Lys-Asp-Glu- |
| | | | | | Leu) endoplasmic |
| | | | | | reticulum protein |
| | | | | | retention receptor 2 |
| PCO1_HUMAN | PCOLCE | NM_002593 | 202097 | 0.52672595 | Procollagen C- |
| | | | | | endopeptidase |
| | | | | | enhancer |
| SPRC_HUMAN | SPARC | NM_003118 | 111779 | 0.51922531 | Secreted protein, |
| | | | | | acidic, cysteine-rich |
| | | | | | (osteonectin) |
| NC5R_HUMAN | DIA1 | NM_007326 | 274464 | 0.51482855 | Diaphorase (NADH) |
| | | | | | (cytochrome b-5 |
| | | | | | reductase) |
| SEPR_HUMAN | FAP | NM_004460 | 418 | 0.51466336 | Fibroblast activation |
| | | | | | protein, alpha |
| HSAC013564 | COL5A1 | NM_000093 | 146428 | 0.51225806 | Collagen, type V, alpha 1 |
| HSAC009848 | ZFP93 | NM_004234 | 298089 | 0.50989391 | Zinc finger protein 93 |
| | | | | | homolog (mouse) |
| PGCV_HUMAN | CSPG2 | U16306 | 81800 | 0.50906177 | Chondroitin sulfate |
| | | | | | proteoglycan 2 |
| | | | | | (versican) |
| Q14521 | LLGL2 | NM_004524 | 3123 | −0.50695888 | Lethal giant larvae |
| | | | | | homolog 2 (Drosophila) |
| CA14_HUMAN | COL4A1 | NM_001845 | 119129 | 0.50664753 | Collagen, type IV, |
| | | | | | alpha 1 |
| HSAC014709 | TEM1 | NM_020404 | 195727 | 0.50647843 | Tumor endothelial |
| | | | | | marker 1 precursor |
| UROK_HUMAN | PLAU | NM_002658 | 77274 | 0.50439834 | Plasminogen activator, |
| | | | | | urokinase |
| Q8N6P7 | IL22R | NM_021258 | 110915 | −0.49928496 | Interleukin 22 receptor |
| LEG1_HUMAN | LGALS1 | NM_002305 | 227751 | 0.48740219 | Lectin, galactoside- |
| | | | | | binding, soluble, 1 |
| | | | | | (galectin 1) |
| CA25_HUMAN | COL5A2 | NM_000393 | 82985 | 0.48675513 | Collagen, type V, alpha 2 |
| CA13_HUMAN | COL3A1 | NM_000090 | 119571 | 0.48459913 | Collagen, type III, |
| | | | | | alpha 1 (Ehlers-Danlos |
| | | | | | syndrome type IV, |
| | | | | | autosomal dominant) |
| Q9BYD5 | LOC84518 | NM_032488 | 148590 | −0.48449285 | Protein related with |
| | | | | | psoriasis |
| BGH3_HUMAN | TGFBI | NM_000358 | 118787 | 0.48290139 | Transforming growth |
| | | | | | factor, beta-induced, |
| | | | | | 68 kD |
| CQT6_HUMAN | CTRP6 | NM_031910 | 22011 | 0.47978614 | Complement-c1q tumor |
| | | | | | necrosis factor-related |
| | | | | | protein 6 |
| AD12_HUMAN | ADAM12 | NM_003474 | 8850 | 0.47905457 | A disintegrin and |
| | | | | | metalloproteinase |
| | | | | | domain 12 (meltrin |
| | | | | | alpha) |
| Q8N3N2 | FLJ11196 | NM_018357 | 6166 | 0.47772636 | Hypothetical protein |
| | | | | | FLJ11196 |
| CATK_HUMAN | CTSK | NM_000396 | 83942 | 0.47734967 | Cathepsin K |
| | | | | | (pycnodysostosis) |
| Q96DR2 | 0 | AK055031 | 44289 | −0.47659518 | Homo sapiens cDNA |
| | | | | | FLJ30469 fis, clone |
| | | | | | BRAWH1000037, |
| | | | | | weakly similar to |
| | | | | | UROKINASE |
| | | | | | PLASMINOGEN |
| | | | | | ACTIVATO |
| P03996 | ACTA2 | NM_001613 | 195851 | 0.47560995 | Actin, alpha 2, smooth |
| | | | | | muscle, aorta |
| K6A2_HUMAN | 0 | AK027727 | 184581 | 0.47396182 | Homo sapiens cDNA |
| | | | | | FLJ14821 fis, clone |
| | | | | | OVARC1000556, |
| | | | | | highly similar to |
| | | | | | RIBOSOMAL |
| | | | | | PROTEIN S6 KINASE |
| | | | | | II |
| Q9HBB0 | THY1 | AK057865 | 125359 | 0.4727028 | Thy-1 cell surface |
| | | | | | antigen |
| TM29_HUMAN | TRIM29 | NM_012101 | 82237 | −0.47250994 | Tripartite motif- |
| | | | | | containing 29 |
| TIM2_HUMAN | 0 | AL110197 | 6441 | 0.47199573 | Homo sapiens mRNA; |
| | | | | | cDNA DKFZp586J021 |
| | | | | | (from clone |
| | | | | | DKFZp586J021) |
| MM02_HUMAN | MMP2 | NM_004530 | 111301 | 0.47026319 | Matrix |
| | | | | | metalloproteinase 2 |
| | | | | | (gelatinase A, 72 kD |
| | | | | | gelatinase, 72 kD type |
| | | | | | IV collagenase) |
| MCA2_HUMAN | JTV1 | NM_006303 | 301613 | 0.46964142 | JTV1 gene |
| CA16_HUMAN | COL6A1 | NM_001848 | 108885 | 0.46942561 | Collagen, type VI, |
| | | | | | alpha 1 |
| EVA1_HUMAN | EVA1 | AF275945 | 116651 | −0.4689165 | Epithelial V-like antigen 1 |
| CA21_HUMAN | COL1A2 | NM_000089 | 179573 | 0.46739585 | Collagen, type I, alpha 2 |
| CA36_HUMAN | COL6A3 | NM_004369 | 80988 | 0.46507048 | Collagen, type VI, |
| | | | | | alpha 3 |
| OPN3_HUMAN | OPN3 | NM_014322 | 279926 | 0.46101056 | Opsin 3 |
| | | | | | (encephalopsin, |
| | | | | | panopsin) |
| Q9UBG0 | KIAA0709 | NM_006039 | 7835 | 0.46012143 | Endocytic receptor |
| | | | | | (macrophage mannose |
| | | | | | receptor family) |
| TPM2_HUMAN | TPM2 | NM_003289 | 300772 | 0.46003075 | Tropomyosin 2 (beta) |
| INVO_HUMAN | IVL | NM_005547 | 157091 | −0.45860578 | Involucrin |
| O88386 | RAB10 | NM_016131 | 236494 | −0.45006586 | RAB10, member RAS |
| | | | | | oncogene family |
| PEPL_HUMAN | PPL | NM_002705 | 74304 | −0.44874989 | Periplakin |
| HSAC002603 | FLJ11036 | NM_018306 | 16740 | −0.44841185 | Hypothetical protein |
| | | | | | FLJ11036 |
| TNR5_HUMAN | TNFRSF5 | NM_001250 | 25648 | −0.44547341 | Tumor necrosis factor |
| | | | | | receptor superfamily, |
| | | | | | member 5 |
| FRIH_HUMAN | FTH1 | AK054816 | 62954 | 0.4396982 | Ferritin, heavy |
| | | | | | polypeptide 1 |
| P4H2_HUMAN | P4HA2 | NM_004199 | 3622 | 0.42478412 | Procollagen-proline, 2- |
| | | | | | oxoglutarate 4- |
| | | | | | dioxygenase (proline 4- |
| | | | | | hydroxylase), alpha |
| | | | | | polypeptide II |
| P09526 | RAP1B | NM_015646 | 156764 | 0.42234208 | RAP1B, member of |
| | | | | | RAS oncogene family |
| PS23_HUMAN | SPUVE | NM_007173 | 25338 | 0.42080095 | Protease, serine, 23 |
| HSAC011159 | 0 | AF009267 | 102238 | −0.46560322 | Homo sapiens clone |
| | | | | | FBA1 Cri-du-chat |
| | | | | | region mRNA |
| SDC2_HUMAN | SDC2 | J04621 | 1501 | 0.45455196 | Syndecan 2 (heparan |
| | | | | | sulfate proteoglycan 1, |
| | | | | | cell surface-associated, |
| | | | | | fibroglycan) |
| HSAC013320 | 0 | AL162069 | 140978 | −0.44362782 | Homo sapiens mRNA; |
| | | | | | cDNA DKFZp762H106 |
| | | | | | (from clone |
| | | | | | DKFZp762H106) |
| TAGL_HUMAN | TAGLN | NM_003186 | 75777 | 0.4376112 | Transgelin |
| MM01_HUMAN | MMP1 | NM_002421 | 83169 | 0.4326825 | Matrix |
| | | | | | metalloproteinase 1 |
| | | | | | (interstitial |
| | | | | | collagenase) |
| P05209 | K-ALPHA-1 | NM_006082 | 334842 | 0.42236541 | Tubulin, alpha, |
| | | | | | ubiquitous |
| TSP2_HUMAN | THBS2 | NM_003247 | 108623 | 0.46791583 | Thrombospondin 2 |
| Q8N789 | DKFZP434K0410 | AL137589 | 152149 | −0.43787021 | Hypothetical protein |
| | | | | | DKFZp434K0410 |
| O60335 | KIAA0594 | AB011166 | 103283 | −0.42578156 | KIAA0594 protein |
| P05209 | K-ALPHA-1 | NM_006082 | 334842 | 0.42277793 | Tubulin, alpha, |
| | | | | | ubiquitous |
| TNI3_HUMAN | TNFAIP3 | NM_006290 | 211600 | 0.408128 | Tumor necrosis factor, |
| | | | | | alpha-induced protein 3 |
| FGR1_HUMAN | FGFR1 | NM_023109 | 748 | 0.43553561 | Fibroblast growth factor |
| | | | | | receptor 1 (fms-related |
| | | | | | tyrosine kinase 2, |
| | | | | | Pfeiffer syndrome) |
| CAD2_HUMAN | CDH2 | NM_001792 | 161 | 0.3922152 | Cadherin 2, type 1, N- |
| | | | | | cadherin (neuronal) |
| TCOF_HUMAN | TCOF1 | NM_000356 | 301266 | 0.38956707 | Treacher Collins- |
| | | | | | Franceschetti |
| | | | | | syndrome 1 |
| O14635 | 0 | AF005082 | 113261 | −0.45637623 | Homo sapiens skin- |
| | | | | | specific protein (xp33) |
| | | | | | mRNA, partial cds |
| GLSK_HUMAN | GLS | NM_014905 | 239189 | 0.43214995 | Glutaminase |
| Q9BRJ6 | MGC11257 | NM_032350 | 334368 | 0.42954566 | Hypothetical protein |
| | | | | | MGC11257 |
| ALK1_HUMAN | SLPI | NM_003064 | 251754 | −0.41872706 | Secretory leukocyte |
| | | | | | protease inhibitor |
| | | | | | (antileukoproteinase) |
| AQP3_HUMAN | AQP3 | NM_004925 | 234642 | −0.42891866 | Aquaporin 3 |
| SPIB_HUMAN | SPIB | NM_003121 | 192861 | −0.41021146 | Spi-B transcription |
| | | | | | factor (Spi-1/PU.1 |
| | | | | | related) |
| P05209 | K-ALPHA-1 | NM_006082 | 334842 | 0.41796901 | Tubulin, alpha, |
| | | | | | ubiquitous |
| DRG1_HUMAN | DRG1 | NM_004147 | 115242 | 0.41879372 | Developmentally |
| | | | | | regulated GTP binding |
| | | | | | protein 1 |
| PHMX_HUMAN | PHEMX | NM_005705 | 271954 | 0.38728757 | Pan-hematopoietic |
| | | | | | expression |
| P05209 | K-ALPHA-1 | NM_006082 | 334842 | 0.41741385 | Tubulin, alpha, |
| | | | | | ubiquitous |
| HSAC018335 | 0 | AL137428 | 306459 | −0.45404252 | Homo sapiens mRNA; |
| | | | | | cDNA |
| | | | | | DKFZp761N1323 (from |
| | | | | | clone |
| | | | | | DKFZp761N1323) |
| POSN_HUMAN | OSF-2 | NM_006475 | 136348 | 0.42814786 | Osteoblast specific |
| | | | | | factor 2 (fasciclin I-like) |
| DHC3_HUMAN | CBR3 | NM_001236 | 154510 | −0.48089013 | Carbonyl reductase 3 |
| NCR2_HUMAN | NCOR2 | NM_006312 | 287994 | 0.42024512 | Nuclear receptor co- |
| | | | | | repressor 2 |
| HSAC015262 | 0 | AK021531 | 224398 | 0.43632187 | Homo sapiens cDNA |
| | | | | | FLJ11469 fis, clone |
| | | | | | HEMBA1001658 |
| Q14113 | AEBP1 | NM_001129 | 118397 | 0.42087649 | AE binding protein 1 |
| TBX2_HUMAN | TBX2 | AK001031 | 322856 | 0.41381789 | T-box 2 |
| CRF_HUMAN | CRH | NM_000756 | 75294 | −0.41353804 | Corticotropin releasing |
| | | | | | hormone |
| Q9NUJ7 | FLJ11323 | NM_018390 | 25625 | −0.43842923 | Hypothetical protein |
| | | | | | FLJ11323 |
| Q96DU1 | AKAP2 | AJ303079 | 42322 | −0.44106809 | A kinase (PRKA) |
| | | | | | anchor protein 2 |
| P05209 | K-ALPHA-1 | NM_006082 | 334842 | 0.40736744 | Tubulin, alpha, |
| | | | | | ubiquitous |
| Q969Y7 | MGC4677 | NM_052871 | 337986 | 0.39455354 | Hypothetical protein |
| | | | | | MGC4677 |
| Q9BXY6 | FLJ13962 | NM_024862 | 330407 | −0.44327759 | Hypothetical protein |
| | | | | | FLJ13962 |
| K1CW_HUMAN | HAIK1 | NM_015515 | 9029 | −0.42594883 | Type I intermediate |
| | | | | | filament cytokeratin |
| HSAC019114 | FLJ22622 | NM_025151 | 324841 | −0.4331912 | Hypothetical protein |
| | | | | | FLJ22622 |
| PGS2_HUMAN | DCN | NM_001920 | 76152 | 0.39882715 | Decorin |
| DCOP_HUMAN | ODC-p | NM_052998 | 91681 | −0.41609162 | Ornithine |
| | | | | | decarboxylase-like |
| | | | | | protein |
| HSAC020747 | 0 | AK056828 | 350748 | −0.40822563 | Homo sapiens cDNA |
| | | | | | FLJ32266 fis, clone |
| | | | | | PROST1000419 |
| P05209 | K-ALPHA-1 | NM_006082 | 334842 | 0.39997295 | Tubulin, alpha, |
| | | | | | ubiquitous |
| Q96F00 | 0 | AK025719 | 251664 | 0.39457176 | Homo sapiens cDNA: |
| | | | | | FLJ22066 fis, clone |
| | | | | | HEP10611 |
| ISK5_HUMAN | SPINK5 | NM_006846 | 331555 | −0.43770884 | Serine protease |
| | | | | | inhibitor, Kazal type, 5 |
| GFR1_HUMAN | GFRA1 | NM_005264 | 105445 | 0.37859516 | GDNF family receptor |
| | | | | | alpha 1 |
| AAF24516 | NUDEL | NM_030808 | 3850 | −0.40726277 | LIS1-interacting protein |
| | | | | | NUDEL; |
| | | | | | endooligopeptidase A |
| P05209 | K-ALPHA-1 | NM_006082 | 334842 | 0.39594293 | Tubulin, alpha, |
| | | | | | ubiquitous |
| O60836 | T1A-2 | NM_013317 | 135150 | 0.37127534 | Lung type-I cell |
| | | | | | membrane-associated |
| | | | | | glycoprotein |
| KLKA_HUMAN | KLK10 | NM_002776 | 69423 | −0.40584943 | Kallikrein 10 |
| Q96KC3 | MGC3047 | NM_032348 | 59384 | 0.39922401 | Hypothetical protein |
| | | | | | MGC3047 |
| O95274 | C4.4A | NM_014400 | 11950 | −0.39942513 | GPI-anchored |
| | | | | | metastasis-associated |
| | | | | | protein homolog |
| HSAC015726 | SERPINB13 | AJ001696 | 241407 | −0.41273549 | Serine (or cysteine) |
| | | | | | proteinase inhibitor, |
| | | | | | clade B (ovalbumin), |
| | | | | | member 13 |
| SEN7_HUMAN | SENP7 | AL136599 | 30443 | −0.37916215 | Sentrin/SUMO-specific |
| | | | | | protease |
| HSAC015968 | RPL34P2 | AL049714 | 247903 | −0.39984188 | Ribosomal protein L34 |
| | | | | | pseudogene 2 |
| IM8B_HUMAN | TIMM8B | NM_012459 | 279915 | −0.39646264 | Translocase of inner |
| | | | | | mitochondrial |
| | | | | | membrane 8 homolog |
| | | | | | B (yeast) |
| P05209 | K-ALPHA-1 | NM_006082 | 334842 | 0.39265415 | Tubulin, alpha, |
| | | | | | ubiquitous |
| CYTB_HUMAN | CSTB | NM_000100 | 695 | −0.39593616 | Cystatin B (stefin B) |
| MMDP_HUMAN | MMD | NM_012329 | 79889 | 0.35585533 | Monocyte to |
| | | | | | macrophage |
| | | | | | differentiation- |
| | | | | | associated |
| Q9H5J1 | PREI3 | NM_015387 | 107942 | −0.39957843 | Preimplantation protein 3 |
| Q9Y283 | INVS | NM_014425 | 104715 | 0.38165914 | Inversin |
| S107_HUMAN | S100A7 | NM_002963 | 112408 | −0.38002947 | S100 calcium binding |
| | | | | | protein A7 (psoriasin 1) |
| SR19_HUMAN | SRP19 | NM_003135 | 2943 | −0.39927981 | Signal recognition |
| | | | | | particle 19 kD |
| MA17_HUMAN | DD96 | NM_005764 | 271473 | −0.38289729 | Epithelial protein up- |
| | | | | | regulated in carcinoma, |
| | | | | | membrane associated |
| | | | | | protein 17 |
| O75943 | RAD17 | NM_002873 | 16184 | −0.38971266 | RAD17 homolog (S. pombe) |
| THA_HUMAN | THRA | NM_003250 | 724 | 0.38475204 | Thyroid hormone |
| | | | | | receptor, alpha |
| | | | | | (erythroblastic |
| | | | | | leukemia viral (v-erb-a) |
| | | | | | oncogene homolog, |
| | | | | | avian) |
| HSAC008967 | 0 | AK021982 | 287465 | 0.38611307 | Homo sapiens cDNA |
| | | | | | FLJ11920 fis, clone |
| | | | | | HEMBB1000312 |
| TFE2_HUMAN | TCF3 | M31523 | 101047 | 0.38678059 | Transcription factor 3 |
| | | | | | (E2A immunoglobulin |
| | | | | | enhancer binding |
| | | | | | factors E12/E47) |
| SUL2_HUMAN | KIAA1247 | AB033073 | 43857 | 0.39182636 | Similar to glucosamine- |
| | | | | | 6-sulfatases |
| HRA3_HUMAN | HTRA3 | AY040094 | 60440 | 0.38043383 | Serine protease |
| | | | | | HTRA3 |
| CN4A_HUMAN | PDE4A | NM_006202 | 89901 | 0.36750244 | Phosphodiesterase 4A, |
| | | | | | cAMP-specific |
| | | | | | (phosphodiesterase E2 |
| | | | | | dunce homolog, |
| | | | | | Drosophila) |
| LTB2_HUMAN | LTBP2 | NM_000428 | 83337 | 0.36424793 | Latent transforming |
| | | | | | growth factor beta |
| | | | | | binding protein 2 |
| CSF2_HUMAN | CSF2 | NM_000758 | 1349 | 0.34759785 | Colony stimulating |
| | | | | | factor 2 (granulocyte- |
| | | | | | macrophage) |
| S109_HUMAN | S100A9 | NM_002965 | 112405 | −0.38115533 | S100 calcium binding |
| | | | | | protein A9 (calgranulin |
| | | | | | B) |
| MAL2_HUMAN | MAL2 | NM_052886 | 76550 | −0.37756452 | Mal, T-cell |
| | | | | | differentiation protein 2 |
| HSAC004288 | LANO | NM_018214 | 35091 | −0.39020801 | LAP (leucine-rich |
| | | | | | repeats and PDZ) and |
| | | | | | no PDZ protein |
| P05209 | K-ALPHA-1 | NM_006082 | 334842 | 0.37816007 | Tubulin, alpha, |
| | | | | | ubiquitous |
| EMP3_HUMAN | EMP3 | NM_001425 | 9999 | 0.37961495 | Epithelial membrane |
| | | | | | protein 3 |
| LUM_HUMAN | LUM | NM_002345 | 79914 | 0.36091717 | Lumican |
| Q8NC43 | FLJ23091 | NM_024911 | 250746 | 0.4002659 | Hypothetical protein |
| | | | | | FLJ23091 |
| HRA1_HUMAN | PRSS11 | NM_002775 | 75111 | 0.38314991 | Protease, serine, 11 |
| | | | | | (IGF binding) |
| CAH6_HUMAN | CA6 | NM_001215 | 100322 | 0.38495881 | Carbonic anhydrase VI |
| SCGF_HUMAN | SCGF | NM_002975 | 105927 | 0.38520465 | Stem cell growth factor; |
| | | | | | lymphocyte secreted |
| | | | | | C-type lectin |
| CALD_HUMAN | CALD1 | NM_033138 | 325474 | 0.36017333 | Caldesmon 1 |
| SYH_HUMAN | HARS | NM_002109 | 77798 | 0.34476889 | Histidyl-tRNA |
| | | | | | synthetase |
| Q8IXQ7 | LABH1 | NM_032604 | 98608 | 0.36012503 | Lung alpha/beta |
| | | | | | hydrolase 1 |
| WEE1_HUMAN | WEE1 | X62048 | 75188 | −0.38967133 | WEE1+ homolog (S. pombe) |
| Q9H0B8 | DKFZP434B044 | NM_031476 | 262958 | 0.36902739 | Hypothetical protein |
| | | | | | DKFZp434B044 |
| M1B1_HUMAN | MAN1B1 | NM_016219 | 279881 | 0.37430439 | Mannosidase, alpha, |
| | | | | | class 1B, member 1 |
| FBX8_HUMAN | FBXO8 | NM_012180 | 76917 | −0.37436238 | F-box only protein 8 |
| SM3C_HUMAN | SEMA3C | NM_006379 | 171921 | 0.35697551 | Sema domain, |
| | | | | | immunoglobulin |
| | | | | | domain (Ig), short basic |
| | | | | | domain, secreted, |
| | | | | | (semaphorin) 3C |
| RB25_HUMAN | CATX-8 | NM_020387 | 150826 | −0.38816294 | CATX-8 protein |
| ROL_HUMAN | HNRPL | NM_001533 | 2730 | 0.34146766 | Heterogeneous nuclear |
| | | | | | ribonucleoprotein L |
| FX37_HUMAN | MGC11279 | NM_024326 | 10915 | −0.38119046 | Hypothetical protein |
| | | | | | MGC11279 |
| HSAC003262 | KIAA0350 | AB002348 | 23263 | −0.39564135 | KIAA0350 protein |
| P05209 | K-ALPHA-1 | NM_006082 | 334842 | 0.37391308 | Tubulin, alpha, |
| | | | | | ubiquitous |
| BTE4_HUMAN | KLF16 | NM_031918 | 303194 | 0.40092333 | Kruppel-like factor 16 |
| MK_HUMAN | MDK | NM_002391 | 82045 | −0.38900237 | Midkine (neurite |
| | | | | | growth-promoting |
| | | | | | factor 2) |
| Q9NRD9 | DUOX1 | NM_017434 | 272813 | −0.3913498 | Dual oxidase 1 |
| P05209 | K-ALPHA-1 | NM_006082 | 334842 | 0.36936704 | Tubulin, alpha, |
| | | | | | ubiquitous |
| Z185_HUMAN | ZNF185 | NM_007150 | 16622 | −0.36378851 | Zinc finger protein 185 |
| | | | | | (LIM domain) |
| TBG2_HUMAN | TUBG2 | NM_016437 | 279669 | 0.34203519 | Tubulin, gamma 2 |
| AAKC_HUMAN | PRKAB2 | NM_005399 | 50732 | −0.35738949 | Protein kinase, AMP- |
| | | | | | activated, beta 2 non- |
| | | | | | catalytic subunit |
| HSAC006508 | COL18A1 | AF018081 | 78409 | 0.37705859 | Collagen, type XVIII, |
| | | | | | alpha 1 |
| Q9BSY6 | ZD52F10 | NM_033317 | 32343 | −0.37419257 | Hypothetical gene |
| | | | | | ZD52F10 |
| SOX4_HUMAN | 0 | AJ420500 | 351928 | −0.41521785 | Homo sapiens mRNA |
| | | | | | full length insert cDNA |
| | | | | | clone EUROIMAGE |
| | | | | | 1977059 |
| P05209 | K-ALPHA-1 | NM_006082 | 334842 | 0.37198391 | Tubulin, alpha, |
| | | | | | ubiquitous |
| MIC2_HUMAN | MIC2 | NM_002414 | 177543 | 0.36316608 | Antigen identified by |
| | | | | | monoclonal antibodies |
| | | | | | 12E7, F21 and O13 |
| VWF_HUMAN | VWF | NM_000552 | 110802 | −0.36187757 | Von Willebrand factor |
| MFA5_HUMAN | MAGP2 | NM_003480 | 300946 | 0.34145531 | Microfibril-associated |
| | | | | | glycoprotein-2 |
| ELAF_HUMAN | PI3 | NM_002638 | 112341 | −0.39367703 | Protease inhibitor 3, |
| | | | | | skin-derived (SKALP) |
| WD13_HUMAN | WDR13 | NM_017883 | 12142 | 0.35576479 | WD repeat domain 13 |
| PCB1_HUMAN | PCBP1 | NM_006196 | 2853 | −0.35199657 | Poly(rC) binding |
| | | | | | protein 1 |
| DYHC_HUMAN | DNCH1 | AB002323 | 7720 | 0.36908919 | Dynein, cytoplasmic, |
| | | | | | heavy polypeptide 1 |
| Q8WUB2 | HSU79274 | NM_013300 | 150555 | −0.41095099 | Protein predicted by |
| | | | | | clone 23733 |
| Q96N74 | PGLYRP | NM_052890 | 282244 | −0.37818832 | Peptidoglycan |
| | | | | | recognition protein L |
| | | | | | precursor |
| HSAC018816 | 0 | AK055723 | 310919 | −0.37825237 | Homo sapiens cDNA |
| | | | | | FLJ31161 fis, clone |
| | | | | | KIDNE1000028 |
| PPL2_HUMAN | PPIL2 | NM_014337 | 93523 | −0.34926253 | Peptidylprolyl |
| | | | | | isomerase |
| | | | | | (cyclophilin)-like 2 |
| HSAC015090 | 0 | AK055294 | 211132 | 0.34738745 | Homo sapiens cDNA |
| | | | | | FLJ30732 fis, clone |
| | | | | | FEBRA2000126, |
| | | | | | weakly similar to Mus |
| | | | | | musculus PDZ domain |
| | | | | | actin |
| P05209 | K-ALPHA-1 | NM_006082 | 334842 | 0.36404497 | Tubulin, alpha, |
| | | | | | ubiquitous |
| DSG3_HUMAN | DSG3 | NM_001944 | 1925 | −0.35916779 | Desmoglein 3 |
| | | | | | (pemphigus vulgaris |
| | | | | | antigen) |
| CTGF_HUMAN | CTGF | NM_001901 | 75511 | 0.36449331 | Connective tissue |
| | | | | | growth factor |
| Q96BW1 | 0 | AK056354 | 91612 | 0.34964326 | Homo sapiens, clone |
| | | | | | MGC: 23937 |
| | | | | | IMAGE: 3930177, |
| | | | | | mRNA, complete cds |
| P05209 | K-ALPHA-1 | NM_006082 | 334842 | 0.36440521 | Tubulin, alpha, |
| | | | | | ubiquitous |
| HSAC020349 | 0 | BC014584 | 348710 | 0.33020586 | Homo sapiens, clone |
| | | | | | IMAGE: 4047062, |
| | | | | | mRNA |
| G3P2_HUMAN | GAPD | NM_002046 | 169476 | 0.36112372 | Glyceraldehyde-3- |
| | | | | | phosphate |
| | | | | | dehydrogenase |
| DES1_HUMAN | DESC1 | NM_014058 | 201877 | −0.37199135 | DESC1 protein |
| PAI2_HUMAN | SERPINB2 | NM_002575 | 75716 | −0.39418824 | Serine (or cysteine) |
| | | | | | proteinase inhibitor, |
| | | | | | clade B (ovalbumin), |
| | | | | | member 2 |
| PYG2_HUMAN | 0 | BC006132 | 172084 | 0.36848424 | Homo sapiens, clone |
| | | | | | IMAGE: 3627860, |
| | | | | | mRNA, partial cds |
| CA17_HUMAN | COL7A1 | NM_000094 | 1640 | 0.35840992 | Collagen, type VII, |
| | | | | | alpha 1 (epidermolysis |
| | | | | | bullosa, dystrophic, |
| | | | | | dominant and |
| | | | | | recessive) |
| Q8NG54 | FLJ21212 | NM_024642 | 47099 | −0.35274616 | Hypothetical protein |
| | | | | | FLJ21212 |
| Q9H6K5 | FLJ22184 | NM_025094 | 288540 | −0.36166463 | Hypothetical protein |
| | | | | | FLJ22184 |
| Q96FP4 | 0 | BC010607 | 104679 | 0.34999129 | Homo sapiens, clone |
| | | | | | MGC: 18216 |
| | | | | | IMAGE: 4156235, |
| | | | | | mRNA, complete cds |
| HSAC019418 | 0 | AK021970 | 333438 | −0.37893723 | Homo sapiens cDNA |
| | | | | | FLJ11908 fis, clone |
| | | | | | HEMBB1000089 |
| HSAC019917 | 0 | AF130069 | 343363 | −0.34377216 | Homo sapiens clone |
| | | | | | FLB8436 PRO2277 |
| | | | | | mRNA, complete cds |
| HSAC003296 | TACSTD2 | NM_002353 | 23582 | −0.3427956 | Tumor-associated |
| | | | | | calcium signal |
| | | | | | transducer 2 |
| TTC7_HUMAN | KIAA1140 | AB032966 | 131728 | 0.34916061 | KIAA1140 protein |
| G3P2_HUMAN | GAPD | NM_002046 | 169476 | 0.35497237 | Glyceraldehyde-3- |
| | | | | | phosphate |
| | | | | | dehydrogenase |
| Q86VR7 | 0 | BC016993 | 293236 | −0.40258581 | Homo sapiens, clone |
| | | | | | IMAGE: 4401841, |
| | | | | | mRNA |
| G3P2_HUMAN | GAPD | NM_002046 | 169476 | 0.35582864 | Glyceraldehyde-3- |
| | | | | | phosphate |
| | | | | | dehydrogenase |
| Q96CG8 | 0 | BC014245 | 283713 | 0.34868937 | Homo sapiens, Similar |
| | | | | | to RIKEN cDNA |
| | | | | | 1110014B07 gene, |
| | | | | | clone MGC: 20766 |
| | | | | | IMAGE: 4586039, |
| | | | | | mRNA, complete c |
| HSAC004760 | 0 | S73288 | 46320 | −0.36573481 | Small proline-rich |
| | | | | | protein SPRK [human, |
| | | | | | odontogenic |
| | | | | | keratocysts, mRNA |
| | | | | | Partial, 317 nt] |
| KLK5_HUMAN | KLK5 | NM_012427 | 50915 | −0.36794706 | Kallikrein 5 |
| SREC_HUMAN | SREC | NM_003693 | 57735 | 0.35910977 | Acetyl LDL receptor; |
| | | | | | SREC |
| Q8IVC7 | EPSTI1 | NM_033255 | 343800 | −0.35253232 | Epithelial stromal |
| | | | | | interaction 1 (breast) |
| HSAC008195 | 0 | AF339799 | 174045 | −0.36573039 | Homo sapiens clone |
| | | | | | IMAGE: 2363394, |
| | | | | | mRNA sequence |
| TPM2_HUMAN | TPM1 | NM_000366 | 77899 | 0.3486721 | Tropomyosin 1 (alpha) |
| Q9UFS8 | AGS3 | AL117478 | 239370 | −0.35552159 | Likely ortholog of rat |
| | | | | | activator of G-protein |
| | | | | | signaling 3 |
| G3P2_HUMAN | GAPD | NM_002046 | 169476 | 0.35209827 | Glyceraldehyde-3- |
| | | | | | phosphate |
| | | | | | dehydrogenase |
| HSAC011719 | CLDN5 | NM_003277 | 110903 | −0.35850977 | Claudin 5 |
| | | | | | (transmembrane |
| | | | | | protein deleted in |
| | | | | | velocardiofacial |
| | | | | | syndrome) |
| G3P2_HUMAN | GAPD | NM_002046 | 169476 | 0.34936984 | Glyceraldehyde-3- |
| | | | | | phosphate |
| | | | | | dehydrogenase |
| HSAC002456 | 0 | AK055225 | 15167 | −0.34350313 | Homo sapiens cDNA |
| | | | | | FLJ30663 fis, clone |
| | | | | | FCBBF1000598, |
| | | | | | moderately similar to |
| | | | | | ZINC FINGER |
| | | | | | PROTEIN 84 |
| P05209 | K-ALPHA-1 | NM_006082 | 334842 | 0.35175871 | Tubulin, alpha, |
| | | | | | ubiquitous |
| HSAC014332 | BENE | U17077 | 185055 | −0.36524246 | BENE protein |
| G3P2_HUMAN | GAPD | NM_002046 | 169476 | 0.34976822 | Glyceraldehyde-3- |
| | | | | | phosphate |
| | | | | | dehydrogenase |
| HSAC020817 | 0 | AK055932 | 350820 | −0.36618612 | Homo sapiens cDNA |
| | | | | | FLJ31370 fis, clone |
| | | | | | NB9N42000122 |
| KLKB_HUMAN | KLK11 | NM_006853 | 57771 | −0.34648295 | Kallikrein 11 |
| AAC1_HUMAN | ACTN1 | NM_001102 | 119000 | 0.33964129 | Actinin, alpha 1 |
| G3P2_HUMAN | GAPD | NM_002046 | 169476 | 0.3514833 | Glyceraldehyde-3- |
| | | | | | phosphate |
| | | | | | dehydrogenase |
| Q8WW05 | 0 | AC006017 | 131311 | −0.34898752 | Homo sapiens PAC |
| | | | | | clone RP5-981O7 from |
| | | | | | 7q34-q36 |
| HAS3_HUMAN | HAS3 | AF232772 | 85962 | −0.37055655 | Hyaluronan synthase 3 |
| HSAC019767 | 0 | AK022838 | 336419 | −0.35771887 | Homo sapiens cDNA |
| | | | | | FLJ12776 fis, clone |
| | | | | | NT2RP2001678 |
| O60565 | CKTSF1B1 | NM_013372 | 40098 | 0.33592448 | Cysteine knot |
| | | | | | superfamily 1, BMP |
| | | | | | antagonist 1 |
| HSAC013123 | DKFZp547D065 | AL390147 | 134742 | 0.35377439 | Hypothetical protein |
| | | | | | DKFZp547D065 |
| G3P2_HUMAN | GAPD | NM_002046 | 169476 | 0.35115594 | Glyceraldehyde-3- |
| | | | | | phosphate |
| | | | | | dehydrogenase |
| Q14707 | KTN1 | NM_004986 | 211577 | 0.34546179 | Kinectin 1 (kinesin |
| | | | | | receptor) |
| HSAC001617 | UCRP | AF388367 | 8201 | −0.32315934 | Usher critical region |
| | | | | | protein pseudogene |
| HSAC017169 | 0 | AL137617 | 274583 | −0.36507656 | Homo sapiens mRNA; |
| | | | | | cDNA |
| | | | | | DKFZp434C0512 (from |
| | | | | | clone |
| | | | | | DKFZp434C0512) |
| Q9H7K0 | 0 | AK024480 | 13766 | −0.35542828 | Homo sapiens mRNA |
| | | | | | for FLJ00074 protein, |
| | | | | | partial cds |
| G3P2_HUMAN | GAPD | NM_002046 | 169476 | 0.34999375 | Glyceraldehyde-3- |
| | | | | | phosphate |
| | | | | | dehydrogenase |
| HSAC015798 | 0 | AK000745 | 243901 | 0.33333793 | Homo sapiens mRNA; |
| | | | | | cDNA |
| | | | | | DKFZp564C1563 (from |
| | | | | | clone |
| | | | | | DKFZp564C1563) |
| CLH1_HUMAN | CLTC | NM_004859 | 178710 | 0.33305718 | Clathrin, heavy |
| | | | | | polypeptide (Hc) |
| PAI1_HUMAN | SERPINE1 | NM_000602 | 82085 | 0.32335137 | Serine (or cysteine) |
| | | | | | proteinase inhibitor, |
| | | | | | clade E (nexin, |
| | | | | | plasminogen activator |
| | | | | | inhibitor type 1), |
| HSAC018152 | 0 | AK057719 | 303105 | −0.33343853 | Homo sapiens cDNA |
| | | | | | FLJ33157 fis, clone |
| | | | | | UTERU2000393 |
| HSAC019360 | 0 | AK024104 | 333154 | −0.33563282 | Homo sapiens cDNA |
| | | | | | FLJ14042 fis, clone |
| | | | | | HEMBA1006038, |
| | | | | | weakly similar to |
| | | | | | LAMININ ALPHA-5 |
| | | | | | CHAIN |
| Q9H7E9 | FLJ20989 | NM_023080 | 169615 | 0.33185124 | Hypothetical protein |
| | | | | | FLJ20989 |
| TGF1_HUMAN | TGFB1 | NM_000660 | 1103 | 0.36859438 | Transforming growth |
| | | | | | factor, beta 1 |
| Q9NRA1 | PDGFC | NM_016205 | 43080 | 0.33440168 | Platelet derived growth |
| | | | | | factor C |
| HSAC020279 | 0 | BC014381 | 348523 | −0.38018709 | Homo sapiens, clone |
| | | | | | IMAGE: 4046329, |
| | | | | | mRNA |
| Q8IXK0 | EDR2 | NM_004427 | 165263 | 0.3333879 | Early development |
| | | | | | regulator 2 |
| | | | | | (polyhomeotic 2 |
| | | | | | homolog) |
| HSAC017377 | PRO0514 | NM_014131 | 278939 | −0.32206704 | PRO0514 protein |
| Q9BPY8 | SMAP31 | NM_032495 | 13775 | −0.39460445 | Hypothetical protein |
| | | | | | SMAP31 |
| MT70_HUMAN | M6A | NM_019852 | 268149 | 0.35349822 | Putative |
| | | | | | methyltransferase |
| UDP2_HUMAN | UGP2 | NM_006759 | 77837 | 0.32421497 | UDP-glucose |
| | | | | | pyrophosphorylase 2 |
| O00205 | SULT2B1 | NM_004605 | 94581 | −0.36104276 | Sulfotransferase family, |
| | | | | | cytosolic, 2B, member 1 |
| Q96A75 | PGS1 | NM_024419 | 278682 | 0.33507703 | Phosphatidylglycerophosphate |
| | | | | | Synthase |
| PI4K_HUMAN | PIK4CA | NM_058004 | 334874 | 0.32401638 | Phosphatidylinositol 4- |
| | | | | | kinase, catalytic, alpha |
| | | | | | polypeptide |
| SPT1_HUMAN | SPINT1 | NM_003710 | 233950 | −0.34424419 | Serine protease |
| | | | | | inhibitor, Kunitz type 1 |
| TGM1_HUMAN | TGM1 | NM_000359 | 22 | −0.36535457 | Transglutaminase 1 (K |
| | | | | | polypeptide epidermal |
| | | | | | type I, protein- |
| | | | | | glutamine-gamma- |
| | | | | | glutamyltransferase) |
| FPGT_HUMAN | FPGT | NM_003838 | 150926 | −0.334427 | Fucose-1-phosphate |
| | | | | | guanylyltransferase |
| Q9H3D4 | TP63 | Y16961 | 137569 | −0.33233956 | Tumor protein 63 kDa |
| | | | | | with strong homology |
| | | | | | to p53 |
| G3P2_HUMAN | GAPD | NM_002046 | 169476 | 0.3376594 | Glyceraldehyde-3- |
| | | | | | phosphate |
| | | | | | dehydrogenase |
| TENA_HUMAN | HXB | NM_002160 | 289114 | 0.31189127 | Hexabrachion |
| | | | | | (tenascin C, cytotactin) |
| OAS1_HUMAN | OAS1 | NM_016816 | 82396 | −0.34392735 | 2′,5′-oligoadenylate |
| | | | | | synthetase 1 (40-46 kD) |
| Q969E4 | MGC15737 | NM_032926 | 39122 | 0.34380675 | Hypothetical protein |
| | | | | | MGC15737 |
| G3P2_HUMAN | GAPD | NM_002046 | 169476 | 0.33818775 | Glyceraldehyde-3- |
| | | | | | phosphate |
| | | | | | dehydrogenase |
| IL6_HUMAN | IL6 | NM_000600 | 93913 | 0.30335363 | Interleukin 6 |
| | | | | | (interferon, beta 2) |
| LMB3_HUMAN | LAMB3 | NM_000228 | 75517 | 0.32051769 | Laminin, beta 3 (nicein |
| | | | | | (125 kD), kalinin |
| | | | | | (140 kD), BM600 |
| | | | | | (125 kD)) |
| HSAC015555 | DKFZp761K1423 | NM_018422 | 236438 | −0.33416824 | Hypothetical protein |
| | | | | | DKFZp761K1423 |
| BASP_HUMAN | BASP1 | NM_006317 | 79516 | 0.3230565 | Brain abundant, |
| | | | | | membrane attached |
| | | | | | signal protein 1 |
| Q8NG12 | FLJ22792 | NM_024921 | 267038 | −0.36618724 | Hypothetical protein |
| | | | | | FLJ22792 |
| Q99603 | TRG@ | AK056843 | 112259 | −0.36306016 | T cell receptor gamma |
| | | | | | locus |
| TDE2_HUMAN | KIAA1253 | AB033079 | 146668 | 0.32859657 | KIAA1253 protein |
| ALC1_HUMAN | IGHM | X58529 | 293441 | 0.30789147 | Immunoglobulin heavy |
| | | | | | constant mu |
| G3P2_HUMAN | GAPD | NM_002046 | 169476 | 0.338632 | Glyceraldehyde-3- |
| | | | | | phosphate |
| | | | | | dehydrogenase |
| KCC1_HUMAN | CAMK1 | NM_003656 | 184402 | 0.32331521 | Calcium/calmodulin- |
| | | | | | dependent protein |
| | | | | | kinase I |
| Q8NCP6 | GLE1L | NM_001499 | 169363 | 0.33812915 | GLE1 RNA export |
| | | | | | mediator-like (yeast) |
| COM5_HUMAN | HT002 | AK023070 | 238928 | 0.31500738 | HT002 protein; |
| | | | | | hypertension-related |
| | | | | | calcium-regulated gene |
| HSAC001787 | SPC18 | NM_014300 | 9534 | 0.32516848 | Signal peptidase |
| | | | | | complex (18 kD) |
| HSAC020210 | 0 | BC012014 | 348340 | 0.32291489 | Homo sapiens, clone |
| | | | | | IMAGE: 4509827, |
| | | | | | mRNA, partial cds |
| Q96AL8 | 0 | BC016969 | 7155 | −0.36007634 | Homo sapiens, clone |
| | | | | | IMAGE: 4428577, |
| | | | | | mRNA, partial cds |
| PEDF_HUMAN | SERPINF1 | NM_002615 | 173594 | 0.3214249 | Serine (or cysteine) |
| | | | | | proteinase inhibitor, |
| | | | | | clade F (alpha-2 |
| | | | | | antiplasmin, pigment |
| | | | | | epithelium derived |
| GA15_HUMAN | DDIT3 | NM_004083 | 337761 | 0.30554219 | DNA-damage-inducible |
| | | | | | transcript 3 |
| NGAL_HUMAN | LCN2 | NM_005564 | 204238 | −0.34145982 | Lipocalin 2 (oncogene |
| | | | | | 24p3) |
| RHOC_HUMAN | ARHC | NM_005167 | 179735 | 0.35111593 | Ras homolog gene |
| | | | | | family, member C |
| G3P2_HUMAN | GAPD | NM_002046 | 169476 | 0.33567842 | Glyceraldehyde-3- |
| | | | | | phosphate |
| | | | | | dehydrogenase |
| O95491 | DNAH11 | NM_003777 | 349084 | −0.37201261 | Dynein, axonemal, |
| | | | | | heavy polypeptide 11 |
| NIF3_HUMAN | NLI-IF | NM_021198 | 283724 | 0.341321 | Nuclear LIM interactor- |
| | | | | | interacting factor |
| Q96MC2 | 0 | AK057222 | 123428 | −0.36359079 | Homo sapiens, Similar |
| | | | | | to CG10958 gene |
| | | | | | product, clone |
| | | | | | MGC: 16372 |
| | | | | | IMAGE: 3929220, |
| | | | | | mRNA, complete cds |
| KVB1_HUMAN | KCNAB1 | AK057059 | 172471 | −0.32465311 | Potassium voltage- |
| | | | | | gated channel, shaker- |
| | | | | | related subfamily, beta |
| | | | | | member 1 |
| P20172 | AP2M1 | NM_004068 | 152936 | 0.33712949 | Adaptor-related protein |
| | | | | | complex 2, mu 1 |
| | | | | | subunit |
| CDX1_HUMAN | CDX1 | NM_001804 | 1545 | 0.32711759 | Caudal type homeo |
| | | | | | box transcription factor 1 |
| G3P2_HUMAN | GAPD | NM_002046 | 169476 | 0.32910404 | Glyceraldehyde-3- |
| | | | | | phosphate |
| | | | | | dehydrogenase |
| LCFD_HUMAN | FACL4 | NM_022977 | 81452 | −0.33567272 | Fatty-acid-Coenzyme A |
| | | | | | ligase, long-chain 4 |
| Q9NX63 | FLJ20420 | NM_017812 | 6693 | −0.34188068 | Hypothetical protein |
| | | | | | FLJ20420 |
| IBP7_HUMAN | IGFBP7 | NM_001553 | 119206 | 0.32895548 | Insulin-like growth |
| | | | | | factor binding protein 7 |
| HSAC018498 | 0 | AK024867 | 306710 | −0.35579721 | Homo sapiens cDNA: |
| | | | | | FLJ21214 fis, clone |
| | | | | | COL00523 |
| HSAC009099 | 0 | AK024849 | 287650 | −0.33929018 | Homo sapiens cDNA: |
| | | | | | FLJ21196 fis, clone |
| | | | | | COL00193 |
| Q8IYN2 | 0 | AK026349 | 288361 | 0.32392521 | Homo sapiens cDNA: |
| | | | | | FLJ22696 fis, clone |
| | | | | | HSI11696 |
| Q96ML0 | 0 | AK056768 | 310758 | −0.36516521 | Homo sapiens cDNA |
| | | | | | FLJ32206 fis, clone |
| | | | | | PLACE6003109 |
| HSAC001114 | 0 | AL137723 | 5855 | −0.35802784 | Homo sapiens mRNA; |
| | | | | | cDNA |
| | | | | | DKFZp434D0818 (from |
| | | | | | clone |
| | | | | | DKFZp434D0818) |
| HSAC019203 | 0 | AF339829 | 326716 | −0.3385585 | Homo sapiens clone |
| | | | | | IMAGE: 609847, mRNA |
| | | | | | sequence |
| IF42_HUMAN | EIF4A2 | NM_001967 | 173912 | 0.32373865 | Eukaryotic translation |
| | | | | | initiation factor 4A, |
| | | | | | isoform 2 |
| HSAC013605 | 0 | AL512697 | 147587 | −0.3486998 | Homo sapiens mRNA; |
| | | | | | cDNA DKFZp547F134 |
| | | | | | (from clone |
| | | | | | DKFZp547F134) |
| Q96N43 | 0 | AK055994 | 28345 | −0.3643947 | Homo sapiens cDNA |
| | | | | | FLJ25084 fis, clone |
| | | | | | CBL08511 |
| O94781 | LAMP3 | NM_014398 | 10887 | −0.3448522 | Lysosomal-associated |
| | | | | | membrane protein 3 |
| Q8N336 | DKFZp547C176 | AL359601 | 48448 | −0.32503335 | Hypothetical protein |
| | | | | | DKFZp547C176 |
| BAC1_HUMAN | BACH1 | NM_001186 | 154276 | −0.34061833 | BTB and CNC |
| | | | | | homology 1, basic |
| | | | | | leucine zipper |
| | | | | | transcription factor 1 |
| G3P2_HUMAN | GAPD | NM_002046 | 169476 | 0.32785053 | Glyceraldehyde-3- |
| | | | | | phosphate |
| | | | | | dehydrogenase |
| FX32_HUMAN | 0 | NM_058229 | 61661 | 0.30830001 | Homo sapiens cDNA |
| | | | | | FLJ32424 fis, clone |
| | | | | | SKMUS2000954, |
| | | | | | moderately similar to |
| | | | | | Homo sapiens F-box |
| | | | | | protein |
| DSC2_HUMAN | DSC2 | NM_004949 | 239727 | −0.34044888 | Desmocollin 2 |
| K2CE_HUMAN | KRT6B | NM_005555 | 335952 | −0.31728346 | Keratin 6B |
| ANXB_HUMAN | ANXA11 | NM_001157 | 75510 | −0.30741089 | Annexin A11 |
| Q9BZC1 | BRUNOL4 | NM_020180 | 41641 | 0.30753295 | Bruno-like 4, RNA |
| | | | | | binding protein |
| | | | | | (Drosophila) |
| MYPH_HUMAN | MYBPH | NM_004997 | 927 | 0.28279614 | Myosin binding protein H |
| ETFA_HUMAN | ETFA | NM_000126 | 169919 | −0.31059939 | Electron-transfer- |
| | | | | | flavoprotein, alpha |
| | | | | | polypeptide (glutaric |
| | | | | | aciduria II) |
| EMP2_HUMAN | EMP2 | NM_001424 | 29191 | −0.31207607 | Epithelial membrane |
| | | | | | protein 2 |
| O95712 | PLA2G4B | NM_005090 | 198161 | −0.37069629 | Phospholipase A2, |
| | | | | | group IVB (cytosolic) |
| HSAC020830 | 0 | AK055817 | 350833 | −0.30730506 | Homo sapiens cDNA |
| | | | | | FLJ31255 fis, clone |
| | | | | | KIDNE2005603, |
| | | | | | moderately similar to 2- |
| | | | | | OXOGLUTARATE |
| | | | | | DEHYDROGENA |
| LOL1_HUMAN | LOXL1 | NM_005576 | 65436 | 0.30369668 | Lysyl oxidase-like 1 |
| HSAC007550 | 0 | AK056805 | 162859 | 0.30195444 | Homo sapiens cDNA |
| | | | | | FLJ32243 fis, clone |
| | | | | | PROST1000039 |
| HPC1_HUMAN | HPCAL1 | NM_002149 | 3618 | −0.3406495 | Hippocalcin-like 1 |
| Q9H5G9 | FLJ23447 | NM_024825 | 175024 | −0.34322107 | Hypothetical protein |
| | | | | | FLJ23447 |
| RSG4_HUMAN | RASAL1 | NM_004658 | 198312 | −0.36721309 | RAS protein activator |
| | | | | | like 1 (GAP1 like) |
| HSAC009467 | 0 | AK021980 | 289068 | 0.28867038 | Homo sapiens cDNA |
| | | | | | FLJ11918 fis, clone |
| | | | | | HEMBB1000272 |
| Q9BXL5 | EDAG-1 | NM_018437 | 176626 | −0.29674005 | Hypothetical protein |
| | | | | | EDAG-1 |
| Q96HM7 | 0 | BC016154 | 350580 | 0.32162091 | Homo sapiens, clone |
| | | | | | MGC: 13247 |
| | | | | | IMAGE: 4040497, |
| | | | | | mRNA, complete cds |
| HSAC014243 | 0 | AK057730 | 184050 | −0.34785224 | Homo sapiens cDNA |
| | | | | | FLJ25001 fis, clone |
| | | | | | CBL00443 |
| Q9NSU6 | 0 | AL137734 | 149356 | −0.31041517 | Homo sapiens mRNA; |
| | | | | | cDNA |
| | | | | | DKFZp586C0721 (from |
| | | | | | clone |
| | | | | | DKFZp586C0721); |
| | | | | | partial cds |
| Q9UHJ4 | KV8.1 | NM_014379 | 13285 | −0.32573332 | Neuronal potassium |
| | | | | | channel alpha subunit |
| SPSY_HUMAN | SMS | NM_004595 | 89718 | 0.30387323 | Spermine synthase |
| HSAC003848 | 0 | AL050204 | 28540 | −0.32209004 | Homo sapiens mRNA; |
| | | | | | cDNA |
| | | | | | DKFZp586F1223 (from |
| | | | | | clone |
| | | | | | DKFZp586F1223) |
| Q8NG17 | 0 | AK057423 | 344530 | −0.32147722 | Homo sapiens cDNA |
| | | | | | FLJ32861 fis, clone |
| | | | | | TESTI2003589 |
| Q9Y6H1 | LOC51142 | NM_016139 | 180859 | 0.28850691 | 16.7 Kd protein |
| Q8TCZ2 | DKFZP761H2024 | NM_031462 | 169388 | 0.29944756 | Hypothetical protein |
| | | | | | DKFZp761H2024 |
| MAT3_HUMAN | MATR3 | NM_018834 | 78825 | 0.33085933 | Matrin 3 |
| TG37_HUMAN | TG737 | NM_006531 | 2291 | −0.31723327 | Probe hTg737 |
| | | | | | (polycystic kidney |
| | | | | | disease, autosomal |
| | | | | | recessive, in) |
| TRIO_HUMAN | TRIO | NM_007118 | 171957 | 0.3112166 | Triple functional |
| | | | | | domain (PTPRF |
| | | | | | interacting) |
| PIMT_HUMAN | PCMT1 | NM_005389 | 79137 | 0.30318248 | Protein-L-isoaspartate |
| | | | | | (D-aspartate) O- |
| | | | | | methyltransferase |
| HSAC018541 | 0 | AK025055 | 306756 | 0.29732767 | Homo sapiens cDNA: |
| | | | | | FLJ21402 fis, clone |
| | | | | | COL03734 |
| FXO4_HUMAN | MLLT7 | NM_005938 | 239663 | −0.3275796 | Myeloid/lymphoid or |
| | | | | | mixed-lineage |
| | | | | | leukemia (trithorax |
| | | | | | homolog, Drosophila); |
| | | | | | translocated to, 7 |
| HSAC005508 | LOC57099 | NM_020371 | 63168 | −0.31221717 | Cell death regulator |
| | | | | | aven |
| HSAC013183 | 0 | AL049328 | 135642 | −0.30015385 | Homo sapiens mRNA; |
| | | | | | cDNA DKFZp564E026 |
| | | | | | (from clone |
| | | | | | DKFZp564E026) |
| JAK1_HUMAN | JAK1 | NM_002227 | 50651 | −0.32933441 | Janus kinase 1 (a |
| | | | | | protein tyrosine kinase) |
| HSAC018490 | 0 | AK024712 | 306702 | −0.32269088 | Homo sapiens cDNA: |
| | | | | | FLJ21059 fis, clone |
| | | | | | CAS00740 |
| NHR1_HUMAN | SLC9A3R1 | NM_004252 | 184276 | −0.32298859 | Solute carrier family 9 |
| | | | | | (sodium/hydrogen |
| | | | | | exchanger), isoform 3 |
| | | | | | regulatory factor 1 |
| HSAC018263 | 0 | AL109730 | 306331 | 0.30249865 | Homo sapiens mRNA |
| | | | | | full length insert cDNA |
| | | | | | clone EUROIMAGE |
| | | | | | 68600 |
| IRK3_HUMAN | KCNJ3 | NM_002239 | 37169 | −0.27575989 | Potassium inwardly- |
| | | | | | rectifying channel, |
| | | | | | subfamily J, member 3 |
| CTR1_HUMAN | 0 | AL050021 | 14846 | −0.29584263 | Homo sapiens mRNA; |
| | | | | | cDNA DKFZp564D016 |
| | | | | | (from clone |
| | | | | | DKFZp564D016) |
| FAN_HUMAN | NSMAF | NM_003580 | 78687 | 0.31613497 | Neutral |
| | | | | | sphingomyelinase (N- |
| | | | | | SMase) activation |
| | | | | | associated factor |
| Q96NA9 | MEG3 | AK055725 | 112844 | 0.25180621 | Maternally expressed 3 |
| HSAC020071 | 0 | AK057520 | 345390 | −0.33442472 | Homo sapiens cDNA |
| | | | | | FLJ32958 fis, clone |
| | | | | | TESTI2008234 |
| EML1_HUMAN | EMAPL | NM_004434 | 12451 | 0.29532372 | Echinoderm |
| | | | | | microtubule-associated |
| | | | | | protein-like |
| O75915 | JWA | NM_006407 | 92384 | 0.3125817 | Vitamin A responsive; |
| | | | | | cytoskeleton related |
| TRKB_HUMAN | 0 | AJ420458 | 351930 | −0.30975194 | Homo sapiens mRNA |
| | | | | | full length insert cDNA |
| | | | | | clone EUROIMAGE |
| | | | | | 1630957 |
| Q9P1S1 | FLJ10116 | NM_018000 | 79741 | −0.30735296 | Hypothetical protein |
| | | | | | FLJ10116 |
| Q96B21 | 0 | BC016153 | 283552 | −0.31888744 | Homo sapiens, Similar |
| | | | | | to hypothetical protein |
| | | | | | FLJ10134, clone |
| | | | | | MGC: 13208 |
| | | | | | IMAGE: 3841102, |
| | | | | | mRNA, complet |
| RB3D_HUMAN | GOV | AF019226 | 8036 | −0.30399168 | Glioblastoma |
| | | | | | overexpressed |
| CGD1_HUMAN | CCND1 | NM_053056 | 82932 | 0.2829885 | Cyclin D1 (PRAD1: |
| | | | | | parathyroid |
| | | | | | adenomatosis 1) |
| NPS1_HUMAN | NIPSNAP1 | NM_003634 | 173878 | 0.29229903 | Nipsnap homolog 1 (C. elegans) |
| COR3_HUMAN | SPRR3 | NM_005416 | 139322 | −0.33687812 | Small proline-rich |
| | | | | | protein 3 |
| LDHA_HUMAN | LDHA | NM_005566 | 2795 | 0.30841977 | Lactate dehydrogenase A |
| KPCI_HUMAN | PRKCI | NM_002740 | 1904 | −0.32314399 | Protein kinase C, iota |
| IF42_HUMAN | EIF4A2 | NM_001967 | 173912 | 0.28965638 | Eukaryotic translation |
| | | | | | initiation factor 4A, |
| | | | | | isoform 2 |
| HSAC009161 | 0 | AK026955 | 287737 | 0.30940785 | Homo sapiens cDNA: |
| | | | | | FLJ23302 fis, clone |
| | | | | | HEP11143 |
| PLE1_HUMAN | PLEC1 | NM_000445 | 79706 | 0.30274257 | Plectin 1, intermediate |
| | | | | | filament binding |
| | | | | | protein, 500 kD |
| HSAC014877 | 0 | BE961032 | 200400 | −0.2926312 | Human DNA sequence |
| | | | | | from BAC 15E1 on |
| | | | | | chromosome 12. |
| | | | | | Contains Cytochrome |
| | | | | | C Oxidase Polypeptide |
| | | | | | VIa-liv |
| O94920 | KIAA0831 | NM_014924 | 103000 | −0.34631714 | KIAA0831 protein |
| ACBP_HUMAN | DBI | NM_020548 | 78888 | −0.31289222 | Diazepam binding |
| | | | | | inhibitor (GABA |
| | | | | | receptor modulator, |
| | | | | | acyl-Coenzyme A |
| | | | | | binding protein) |
| HSAC000440 | HBP17 | NM_005130 | 1690 | −0.28350117 | Heparin-binding growth |
| | | | | | factor binding protein |
| MY10_HUMAN | MYO10 | NM_012334 | 61638 | 0.28091339 | Myosin X |
| CP2B_HUMAN | CYP27B1 | NM_000785 | 199270 | 0.28232127 | Cytochrome P450, |
| | | | | | subfamily XXVIIB (25- |
| | | | | | hydroxyvitamin D-1- |
| | | | | | alpha-hydroxylase), |
| | | | | | polypeptide 1 |
| HSAC007525 | 0 | AL133568 | 161454 | 0.27850204 | Homo sapiens mRNA; |
| | | | | | cDNA DKFZp434N197 |
| | | | | | (from clone |
| | | | | | DKFZp434N197) |
| Q9BV47 | MGC1136 | NM_024025 | 8719 | 0.27658422 | Hypothetical protein |
| | | | | | MGC1136 |
| IHA_HUMAN | INHA | NM_002191 | 1734 | 0.28237705 | Inhibin, alpha |
| COXM_HUMAN | COX7B | NM_001866 | 75752 | −0.29046875 | Cytochrome c oxidase |
| | | | | | subunit VIIb |
| SM4D_HUMAN | SEMA4D | NM_006378 | 79089 | −0.29092767 | Sema domain, |
| | | | | | immunoglobulin |
| | | | | | domain (Ig), |
| | | | | | transmembrane |
| | | | | | domain (TM) and short |
| | | | | | cytoplasmic domain, |
| | | | | | (se |
| SZ06_HUMAN | SCYB6 | NM_002993 | 164021 | 0.25934353 | Small inducible |
| | | | | | cytokine subfamily B |
| | | | | | (Cys-X-Cys), member |
| | | | | | 6 (granulocyte |
| | | | | | chemotactic protein 2) |
| DTNA_HUMAN | 0 | AK054766 | 351299 | 0.25246747 | Homo sapiens cDNA |
| | | | | | FLJ30204 fis, clone |
| | | | | | BRACE2001496 |
| Q9H668 | FLJ22559 | NM_024928 | 273387 | 0.27961608 | Hypothetical protein |
| | | | | | FLJ22559 |
| LDHA_HUMAN | LDHA | NM_005566 | 2795 | 0.30032227 | Lactate dehydrogenase A |
| LDHA_HUMAN | LDHA | NM_005566 | 2795 | 0.30160612 | Lactate dehydrogenase A |
| Q8NAZ8 | MAIL | NM_031419 | 301183 | −0.32420971 | Molecule possessing |
| | | | | | ankyrin repeats |
| | | | | | induced by |
| | | | | | lipopolysaccharide |
| | | | | | (MAIL), homolog of |
| | | | | | mouse |
| CBX3_HUMAN | CBX3 | NM_016587 | 278554 | 0.28827974 | Chromobox homolog 3 |
| | | | | | (HP1 gamma homolog, |
| | | | | | Drosophila) |
| CLF6_HUMAN | FLJ20396 | NM_017801 | 283685 | 0.30443015 | Hypothetical protein |
| | | | | | FLJ20396 |
| Q9Y4H4 | C6orf9 | NM_022107 | 288316 | 0.3070718 | Chromosome 6 open |
| | | | | | reading frame 9 |
| HSAC018947 | FLJ10257 | AK001119 | 321149 | 0.25119607 | Hypothetical protein |
| | | | | | FLJ10257 |
| O60687 | SRPUL | NM_014467 | 126782 | 0.3005592 | Sushi-repeat protein |
| ETV6_HUMAN | ETV6 | NM_001987 | 169081 | 0.28814409 | Ets variant gene 6 |
| | | | | | (TEL oncogene) |
| BIR2_HUMAN | BIRC2 | NM_001166 | 289107 | 0.27998085 | Baculoviral IAP repeat- |
| | | | | | containing 2 |
| IRS2_HUMAN | IRS2 | AF073310 | 143648 | 0.26611452 | Insulin receptor |
| | | | | | substrate 2 |
| L10K_HUMAN | HSPC023 | NM_014047 | 279945 | 0.28479353 | HSPC023 protein |
| HSAC017376 | 0 | BF541376 | 278937 | 0.27795661 | ESTs, Weakly similar |
| | | | | | to FRHUL ferritin light |
| | | | | | chain [H. sapiens] |
| LDHA_HUMAN | LDHA | NM_005566 | 2795 | 0.29743949 | Lactate dehydrogenase A |
| D103_HUMAN | DEFB3 | NM_018661 | 283082 | −0.32396031 | Defensin, beta 3 |
| Q9BUC9 | DAP | NM_004394 | 75189 | 0.30044109 | Death-associated |
| | | | | | protein |
| Q9BSU0 | FLJ22457 | NM_024901 | 238707 | −0.29867151 | Hypothetical protein |
| | | | | | FLJ22457 |
| LDHA_HUMAN | LDHA | NM_005566 | 2795 | 0.29428289 | Lactate dehydrogenase A |
| TPTE_HUMAN | TPTE | NM_013315 | 122986 | 0.29338232 | Transmembrane |
| | | | | | phosphatase with |
| | | | | | tensin homology |
| HSAC018269 | 0 | AL110206 | 306339 | 0.29149163 | Homo sapiens mRNA; |
| | | | | | cDNA |
| | | | | | DKFZp586N2022 (from |
| | | | | | clone |
| | | | | | DKFZp586N2022) |
| Q8WWY3 | PRPF31 | NM_015629 | 183438 | 0.28992597 | PRP31 pre-mRNA |
| | | | | | processing factor 31 |
| | | | | | homolog (yeast) |
| Q9NWG1 | SDCCAG1 | NM_004713 | 54900 | 0.27325851 | Serologically defined |
| | | | | | colon cancer antigen 1 |
| ID1_HUMAN | ID1 | NM_002165 | 75424 | −0.30300715 | Inhibitor of DNA |
| | | | | | binding 1, dominant |
| | | | | | negative helix-loop- |
| | | | | | helix protein |
| HSAC018258 | 0 | AL080208 | 306325 | −0.32407958 | Homo sapiens mRNA; |
| | | | | | cDNA |
| | | | | | DKFZp586C1523 (from |
| | | | | | clone |
| | | | | | DKFZp586C1523) |
| E2F5_HUMAN | E2F5 | NM_001951 | 2331 | −0.32253572 | E2F transcription factor |
| | | | | | 5, p130-binding |
| GPX7_HUMAN | CL683 | NM_015696 | 43728 | 0.27876425 | Weakly similar to |
| | | | | | glutathione peroxidase 2 |
| AMD1_HUMAN | AMPD1 | NM_000036 | 89570 | 0.30648359 | Adenosine |
| | | | | | monophosphate |
| | | | | | deaminase 1 (isoform |
| | | | | | M) |
| SDC1_HUMAN | SDC1 | NM_002997 | 82109 | −0.29681828 | Syndecan 1 |
| HSAC010708 | FLJ10852 | NM_019028 | 95744 | −0.27932774 | Hypothetical protein |
| | | | | | similar to ankyrin |
| | | | | | repeat-containing |
| | | | | | priotein AKR1 |
| HSAC016387 | 0 | AK023404 | 255890 | −0.30644116 | Homo sapiens cDNA |
| | | | | | FLJ13342 fis, clone |
| | | | | | OVARC1001950 |
| MU81_HUMAN | MUS81 | NM_025128 | 288798 | 0.30225775 | MUS81 endonuclease |
| LDHA_HUMAN | LDHA | NM_005566 | 2795 | 0.29433701 | Lactate dehydrogenase A |
| NFC2_HUMAN | NFATC2 | NM_012340 | 248037 | −0.28597821 | Nuclear factor of |
| | | | | | activated T-cells, |
| | | | | | cytoplasmic, |
| | | | | | calcineurin-dependent 2 |
| CAV2_HUMAN | CAV2 | NM_001233 | 139851 | 0.28575252 | Caveolin 2 |
| HSAC012923 | 0 | AK024243 | 130874 | −0.30558024 | Homo sapiens cDNA |
| | | | | | FLJ14181 fis, clone |
| | | | | | NT2RP2004300 |
| RAG2_HUMAN | RAG2 | AF080577 | 159376 | 0.27054284 | Recombination |
| | | | | | activating gene 2 |
| HSAC001602 | TOM1L2 | AK055959 | 8125 | −0.30209679 | Target of myb1-like 2 |
| | | | | | (chicken) |
| HSAC013938 | FLJ11585 | NM_023075 | 154145 | −0.27045129 | Hypothetical protein |
| | | | | | FLJ11585 |
| WNT3_HUMAN | WNT3 | NM_030753 | 224667 | −0.22616384 | Wingless-type MMTV |
| | | | | | integration site family, |
| | | | | | member 3 |
| P02570 | ACTB | NM_001101 | 288061 | 0.30930373 | Actin, beta |
| Y469_HUMAN | KIAA0469 | NM_014851 | 7764 | −0.27875891 | KIAA0469 gene |
| | | | | | product |
| ALDR_HUMAN | AKR1B1 | NM_001628 | 75313 | 0.24791515 | Aldo-keto reductase |
| | | | | | family 1, member B1 |
| | | | | | (aldose reductase) |
| HSAC003731 | 0 | AK023559 | 27091 | −0.30440343 | Homo sapiens cDNA |
| | | | | | FLJ13497 fis, clone |
| | | | | | PLACE1004518 |
| Q9Y605 | PGR1 | NM_033296 | 285902 | 0.23312692 | T-cell activation protein |
| KFP3_HUMAN | KIFAP3 | NM_014970 | 171374 | 0.29438383 | Kinesin-associated |
| | | | | | protein 3 |
| LDHA_HUMAN | LDHA | NM_005566 | 2795 | 0.29008023 | Lactate dehydrogenase A |
| HSAC016288 | 0 | AL080073 | 251414 | −0.27036219 | Homo sapiens mRNA; |
| | | | | | cDNA |
| | | | | | DKFZp564B1462 (from |
| | | | | | clone |
| | | | | | DKFZp564B1462) |
| HSAC003517 | 0 | AL133611 | 25362 | −0.29482254 | Homo sapiens mRNA; |
| | | | | | cDNA |
| | | | | | DKFZp434O1317 (from |
| | | | | | clone |
| | | | | | DKFZp434O1317) |
| CPG2_HUMAN | LOC51137 | NM_016128 | 102950 | −0.27178678 | Coat protein gamma- |
| | | | | | cop |
| TS22_HUMAN | TSC22 | AK027071 | 114360 | 0.2800433 | Transforming growth |
| | | | | | factor beta-stimulated |
| | | | | | protein TSC-22 |
| S108_HUMAN | S100A8 | NM_002964 | 100000 | −0.30596008 | S100 calcium binding |
| | | | | | protein A8 (calgranulin |
| | | | | | A) |
| GLGB_HUMAN | GBE1 | NM_000158 | 1691 | −0.28462395 | Glucan (1,4-alpha-), |
| | | | | | branching enzyme 1 |
| | | | | | (glycogen branching |
| | | | | | enzyme, Andersen |
| | | | | | disease, glycogen |
| | | | | | stora |
| TCPZ_HUMAN | CCT6A | NM_001762 | 82916 | 0.26946642 | Chaperonin containing |
| | | | | | TCP1, subunit 6A (zeta |
| | | | | | 1) |
| NUDM_HUMAN | NDUFA10 | NM_004544 | 198271 | 0.26779732 | NADH dehydrogenase |
| | | | | | (ubiquinone) 1 alpha |
| | | | | | subcomplex, 10 (42 kD) |
| Q9P005 | HSPC159 | NM_014181 | 114771 | −0.29780343 | HSPC159 protein |
| HSAC013665 | 0 | BC009462 | 149596 | −0.30540195 | Homo sapiens, Similar |
| | | | | | to RIKEN cDNA |
| | | | | | 2310038H17 gene, |
| | | | | | clone MGC: 15974 |
| | | | | | IMAGE: 3542748, |
| | | | | | mRNA, complete c |
| LDHA_HUMAN | LDHA | NM_005566 | 2795 | 0.29038588 | Lactate dehydrogenase A |
| Q96B64 | C1orf29 | NM_006820 | 75470 | −0.28141634 | Chromosome 1 open |
| | | | | | reading frame 29 |
| OSB2_HUMAN | OSBP2 | AF288741 | 7740 | −0.2741057 | Oxysterol binding |
| | | | | | protein 2 |
| IMD2_HUMAN | IMPDH2 | NM_000884 | 75432 | 0.25505345 | IMP (inosine |
| | | | | | monophosphate) |
| | | | | | dehydrogenase 2 |
| NID2_HUMAN | NID2 | NM_007361 | 82733 | 0.2691638 | Nidogen 2 |
| HSAC019850 | 0 | AJ420542 | 339283 | −0.30808045 | Homo sapiens mRNA |
| | | | | | full length insert cDNA |
| | | | | | clone EUROIMAGE |
| | | | | | 1090104 |
| Q8WW54 | LOC51326 | NM_016632 | 264509 | 0.27687084 | ARF protein |
| ELM1_HUMAN | ELMO1 | AL136787 | 31463 | −0.27259252 | Engulfment and cell |
| | | | | | motility 1 (ced-12 |
| | | | | | homolog, C. elegans) |
| HSAC014487 | KIAA1733 | AB051520 | 191979 | −0.26781459 | KIAA1733 protein |
| ENOB_HUMAN | ENO3 | NM_001976 | 118804 | 0.26094659 | Enolase 3, (beta, |
| | | | | | muscle) |
| LDHA_HUMAN | LDHA | NM_005566 | 2795 | 0.29558421 | Lactate dehydrogenase A |
| OM07_HUMAN | LOC54543 | AK027539 | 112318 | 0.28422476 | 6.2 kd protein |
| HSAC003197 | 0 | AK025726 | 22867 | −0.28883729 | Homo sapiens cDNA: |
| | | | | | FLJ22073 fis, clone |
| | | | | | HEP11868 |
| Q92922 | SMARCC1 | NM_003074 | 172280 | −0.28726269 | SWI/SNF related, |
| | | | | | matrix associated, actin |
| | | | | | dependent regulator of |
| | | | | | chromatin, subfamily c, |
| | | | | | member 1 |
| Q9Y2F1 | KIAA0942 | NM_015310 | 6763 | −0.28114819 | KIAA0942 protein |
| SIVA_HUMAN | SIVA | NM_006427 | 112058 | 0.2653505 | CD27-binding (Siva) |
| | | | | | protein |
| LMA3_HUMAN | LAMA3 | NM_000227 | 83450 | 0.25310579 | Laminin, alpha 3 |
| | | | | | (nicein (150 kD), kalinin |
| | | | | | (165 kD), BM600 |
| | | | | | (150 kD), epilegrin) |
| HSAC010931 | 0 | AK054565 | 99014 | 0.2461665 | Homo sapiens, clone |
| | | | | | IMAGE: 3632168, |
| | | | | | mRNA |
| ALM1_HUMAN | ABLIM | NM_002313 | 158203 | −0.29500492 | Actin binding LIM |
| | | | | | protein |
| TKNK_HUMAN | TAC3 | NM_013251 | 9730 | 0.2856172 | Tachykinin 3 |
| | | | | | (neuromedin K, |
| | | | | | neurokinin beta) |
| HPS3_HUMAN | HPS3 | AY033141 | 282804 | −0.32252959 | Hermansky-Pudlak |
| | | | | | syndrome 3 |
| HSAC021198 | 0 | AK055550 | 351640 | −0.2982647 | Homo sapiens cDNA |
| | | | | | FLJ30988 fis, clone |
| | | | | | HLUNG1000030 |
| Q9H657 | FLJ22593 | NM_024703 | 101265 | 0.28723669 | Hypothetical protein |
| | | | | | FLJ22593 |
| Q14244 | MAP7 | NM_003980 | 146388 | −0.26283399 | Microtubule-associated |
| | | | | | protein 7 |
| SPB7_HUMAN | SERPINB7 | NM_003784 | 138202 | −0.33063598 | Serine (or cysteine) |
| | | | | | proteinase inhibitor, |
| | | | | | clade B (ovalbumin), |
| | | | | | member 7 |
| TR1A_HUMAN | TNFRSF1A | NM_001065 | 159 | 0.28778288 | Tumor necrosis factor |
| | | | | | receptor superfamily, |
| | | | | | member 1A |
| TPP2_HUMAN | TPP2 | NM_003291 | 1117 | −0.28378443 | Tripeptidyl peptidase II |
| RU17_HUMAN | SNRP70 | NM_003089 | 174051 | 0.30742876 | Small nuclear |
| | | | | | ribonucleoprotein 70 kD |
| | | | | | polypeptide (RNP |
| | | | | | antigen) |
| RB6A_HUMAN | RAB6A | AK057157 | 5636 | −0.3002715 | RAB6A, member RAS |
| | | | | | oncogene family |
| NDKB_HUMAN | NME2 | NM_002512 | 275163 | 0.27328053 | Non-metastatic cells 2, |
| | | | | | protein (NM23B) |
| | | | | | expressed in |
| KU70_HUMAN | G22P1 | NM_001469 | 197345 | 0.27324664 | Thyroid autoantigen |
| | | | | | 70 kD (Ku antigen) |
| Q8IVU3 | FLJ20637 | NM_017912 | 179669 | −0.27130275 | Hypothetical protein |
| | | | | | FLJ20637 |
| TPSN_HUMAN | TAPBP | AF029750 | 179600 | −0.26528001 | TAP binding protein |
| | | | | | (tapasin) |
| ZEP1_HUMAN | HIVEP1 | NM_002114 | 306 | 0.25935225 | Human |
| | | | | | immunodeficiency virus |
| | | | | | type I enhancer binding |
| | | | | | protein 1 |
| Q9H8B3 | 0 | AK023854 | 154751 | −0.28222739 | Homo sapiens cDNA |
| | | | | | FLJ13792 fis, clone |
| | | | | | THYRO1000072, |
| | | | | | weakly similar to |
| | | | | | MYOSIN LIGHT |
| | | | | | CHAIN KINASE, SMO |
| HSAC018635 | 0 | AK026731 | 306873 | −0.26370149 | Homo sapiens cDNA: |
| | | | | | FLJ23078 fis, clone |
| | | | | | LNG05870 |
| HSAC018455 | 0 | AK024098 | 306663 | −0.25828945 | Homo sapiens cDNA |
| | | | | | FLJ14036 fis, clone |
| | | | | | HEMBA1004709 |
| ECG2_HUMAN | ECG2 | NM_032566 | 244569 | −0.36634539 | Esophagus cancer- |
| | | | | | related gene-2 |
| DLK_HUMAN | DLK1 | NM_003836 | 169228 | −0.29226303 | Delta-like 1 homolog |
| | | | | | (Drosophila) |
| LDHA_HUMAN | LDHA | NM_005566 | 2795 | 0.29093325 | Lactate dehydrogenase A |
| CRTC_HUMAN | CALR | NM_004343 | 16488 | 0.26036309 | Calreticulin |
| HSAC013371 | LOC51087 | NM_015982 | 142989 | 0.2559957 | Germ cell specific Y- |
| | | | | | box binding protein |
| HSAC011127 | 0 | AL133645 | 101651 | −0.30011435 | Homo sapiens mRNA; |
| | | | | | cDNA DKFZp434C107 |
| | | | | | (from clone |
| | | | | | DKFZp434C107) |
| BAA09603 | DJ-1 | NM_007262 | 10958 | 0.2951662 | RNA-binding protein |
| | | | | | regulatory subunit |
| ATF7_HUMAN | ATF7 | NM_006856 | 55888 | −0.28947132 | Activating transcription |
| | | | | | factor 7 |
| RHG6_HUMAN | ARHGAP6 | NM_001174 | 250830 | −0.28626769 | Rho GTPase activating |
| | | | | | protein 6 |
| EPOR_HUMAN | EPOR | NM_000121 | 89548 | −0.27351061 | Erythropoietin receptor |
| SN23_HUMAN | SNAP23 | BC003686 | 184376 | 0.26960802 | Synaptosomal- |
| | | | | | associated protein, |
| | | | | | 23 kD |
| HSAC018299 | 0 | AK000293 | 306391 | −0.26323331 | Homo sapiens cDNA |
| | | | | | FLJ20286 fis, clone |
| | | | | | HEP04358 |
| AMD_HUMAN | PAM | NM_000919 | 83920 | 0.2573707 | Peptidylglycine alpha- |
| | | | | | amidating |
| | | | | | monooxygenase |
| MAGC_HUMAN | MAGEA12 | NM_005367 | 169246 | 0.23080255 | Melanoma antigen, |
| | | | | | family A, 12 |
| HSAC011887 | 0 | AK021693 | 113660 | 0.28054849 | Homo sapiens cDNA |
| | | | | | FLJ11631 fis, clone |
| | | | | | HEMBA1004267 |
| HSAC002243 | 0 | U80766 | 13252 | 0.28054079 | Human EST clone |
| | | | | | 22453 mariner |
| | | | | | transposon Hsmar1 |
| | | | | | sequence |
| LDHA_HUMAN | LDHA | NM_005566 | 2795 | 0.28016177 | Lactate dehydrogenase A |
| HSAC015540 | 0 | AK021431 | 235543 | −0.27184922 | Homo sapiens cDNA |
| | | | | | FLJ11369 fis, clone |
| | | | | | HEMBA1000338 |
| VTNC_HUMAN | VTN | NM_000638 | 2257 | 0.26296734 | Vitronectin (serum |
| | | | | | spreading factor, |
| | | | | | somatomedin B, |
| | | | | | complement S-protein) |
| RS4Y_HUMAN | RPS4Y | NM_001008 | 180911 | 0.25349604 | Ribosomal protein S4, |
| | | | | | Y-linked |
| Q9NXV4 | FLJ20038 | AL117436 | 72071 | 0.2341145 | Hypothetical protein |
| | | | | | FLJ20038 |
| LDHA_HUMAN | LDHA | NM_005566 | 2795 | 0.28721692 | Lactate dehydrogenase A |
| RT36_HUMAN | MRPS36 | NM_033281 | 41182 | −0.27224936 | Mitochondrial |
| | | | | | ribosomal protein S36 |
| HSAC016670 | 0 | AF127771 | 269645 | −0.24620011 | Homo sapiens cell-line |
| | | | | | E8CASS clone E24L |
| | | | | | estradiol-induced |
| | | | | | mRNA sequence |
| HS71_HUMAN | HSPA1A | NM_005345 | 8997 | 0.23168516 | Heat shock 70 kD |
| | | | | | protein 1A |
| SPCQ_HUMAN | SPTBN4 | NM_025213 | 32706 | −0.3053758 | Spectrin, beta, non- |
| | | | | | erythrocytic 4 |
| Q00007 | PPP2R2A | NM_002717 | 179574 | −0.29293379 | Protein phosphatase 2 |
| | | | | | (formerly 2A), |
| | | | | | regulatory subunit B |
| | | | | | (PR 52), alpha isoform |
| P02570 | ACTB | NM_001101 | 288061 | 0.28900489 | Actin, beta |
| PSS1_HUMAN | PTDSS1 | NM_014754 | 77329 | 0.27454166 | Phosphatidylserine |
| | | | | | synthase 1 |
| RBP1_HUMAN | RALBP1 | NM_006788 | 75447 | −0.26200162 | RalA binding protein 1 |
| Q86WL8 | 0 | AK055310 | 65771 | −0.25500063 | Homo sapiens cDNA |
| | | | | | FLJ30748 fis, clone |
| | | | | | FEBRA2000399, |
| | | | | | moderately similar to |
| | | | | | ZINC FINGER |
| | | | | | PROTEIN 45 |
| HSAC017300 | 0 | AL137712 | 278565 | 0.24979685 | Homo sapiens mRNA; |
| | | | | | cDNA |
| | | | | | DKFZp434H0923 (from |
| | | | | | clone |
| | | | | | DKFZp434H0923) |
| SPL1_HUMAN | SPARCL1 | NM_004684 | 75445 | 0.26009914 | SPARC-like 1 (mast9, |
| | | | | | hevin) |
| Q13018 | PLA2R1 | U17033 | 171945 | −0.25693813 | Phospholipase A2 |
| | | | | | receptor 1, 180 kD |
| Q9NPE2 | NEUGRIN | NM_016645 | 323467 | 0.23781654 | Mesenchymal stem cell |
| | | | | | protein DSC92 |
| Q9H652 | MGC4171 | NM_024307 | 289015 | −0.32024804 | Hypothetical protein |
| | | | | | MGC4171 |
| K22E_HUMAN | KRT2A | NM_000423 | 707 | −0.2888545 | Keratin 2A (epidermal |
| | | | | | ichthyosis bullosa of |
| | | | | | Siemens) |
| KPT2_HUMAN | PCTK2 | NM_002595 | 183302 | −0.28303243 | PCTAIRE protein |
| | | | | | kinase 2 |
| Q96B23 | 0 | BC016149 | 33862 | −0.27418789 | Homo sapiens, clone |
| | | | | | MGC: 12909 |
| | | | | | IMAGE: 4040087, |
| | | | | | mRNA, complete cds |
| DAF_HUMAN | DAF | NM_000574 | 1369 | −0.27270806 | Decay accelerating |
| | | | | | factor for complement |
| | | | | | (CD55, Cromer blood |
| | | | | | group system) |
| Q09753 | DEFB1 | NM_005218 | 32949 | −0.26188185 | Defensin, beta 1 |
| HSAC001158 | 0 | AK026673 | 6127 | 0.25225961 | Homo sapiens cDNA: |
| | | | | | FLJ23020 fis, clone |
| | | | | | LNG00943 |
| DECR_HUMAN | DECR1 | NM_001359 | 81548 | 0.24937384 | 2,4-dienoyl CoA |
| | | | | | reductase 1, |
| | | | | | mitochondrial |
| CIT2_HUMAN | CITED2 | AF109161 | 82071 | −0.24627852 | Cbp/p300-interacting |
| | | | | | transactivator, with |
| | | | | | Glu/Asp-rich carboxy- |
| | | | | | terminal domain, 2 |
| IPO4_HUMAN | FLJ23338 | NM_024658 | 61790 | 0.21814335 | Hypothetical protein |
| | | | | | FLJ23338 |
| HSAC009416 | 0 | AK023616 | 288949 | 0.27291917 | Homo sapiens cDNA |
| | | | | | FLJ13554 fis, clone |
| | | | | | PLACE1007478 |
| SPEE_HUMAN | SRM | NM_003132 | 76244 | 0.26205253 | Spermidine synthase |
| GLT2_HUMAN | GALNT2 | NM_004481 | 130181 | 0.25166558 | UDP-N-acetyl-alpha-D- |
| | | | | | galactosamine:polypeptide |
| | | | | | N- |
| | | | | | acetylgalactosaminyltransferase |
| | | | | | 2 (GalNAc- |
| | | | | | T2) |
| EF1G_HUMAN | EEF1G | BC004215 | 2186 | 0.24251596 | Eukaryotic translation |
| | | | | | elongation factor 1 |
| | | | | | gamma |
| IF42_HUMAN | EIF4A2 | NM_001967 | 173912 | 0.22487428 | Eukaryotic translation |
| | | | | | initiation factor 4A, |
| | | | | | isoform 2 |
| Q9P221 | FLJ20654 | AB040940 | 5131 | −0.21966498 | Hypothetical protein |
| | | | | | FLJ20654 |
| HSAC005138 | KIAA0303 | AB002301 | 54985 | −0.27897249 | KIAA0303 protein |
| KLK7_HUMAN | KLK7 | NM_005046 | 151254 | −0.27760264 | Kallikrein 7 |
| | | | | | (chymotryptic, stratum |
| | | | | | corneum) |
| ITP1_HUMAN | ICAP-1A | NM_004763 | 173274 | 0.24572607 | Integrin cytoplasmic |
| | | | | | domain-associated |
| | | | | | protein 1 |
| CT26_HUMAN | C20orf26 | AL117439 | 302122 | −0.30839956 | Chromosome 20 open |
| | | | | | reading frame 26 |
| MK06_HUMAN | MAPK6 | NM_002748 | 271980 | −0.26984777 | Mitogen-activated |
| | | | | | protein kinase 6 |
| HSAC004393 | 0 | AF144233 | 37372 | −0.26241142 | Homo sapiens DNA |
| | | | | | binding peptide mRNA, |
| | | | | | partial cds |
| UBC3_HUMAN | CDC34 | NM_004359 | 76932 | 0.25377891 | Cell division cycle 34 |
| HSAC014234 | 0 | AK056507 | 183953 | −0.24069092 | Homo sapiens cDNA |
| | | | | | FLJ31945 fis, clone |
| | | | | | NT2RP7006980 |
| BRAF_HUMAN | BRAF | NM_004333 | 622 | 0.23815317 | V-raf murine sarcoma |
| | | | | | viral oncogene |
| | | | | | homolog B1 |
| HSAC019969 | COE2 | AK001144 | 343814 | 0.23670517 | Similar to |
| | | | | | TRANSCRIPTION |
| | | | | | FACTOR COE2 |
| | | | | | (EARLY B-CELL |
| | | | | | FACTOR 2) (EBF-2) |
| | | | | | (OLF-1/EBF-LIKE 3) |
| | | | | | (OE-3) (O/E- |
| HSAC019505 | 0 | AY039026 | 334395 | −0.18416075 | Homo sapiens |
| | | | | | immunoglobulin mu |
| | | | | | chain antibody MO30 |
| | | | | | (IgM) mRNA, complete |
| | | | | | cds |
| EMP1_HUMAN | EMP1 | NM_001423 | 79368 | −0.30522881 | Epithelial membrane |
| | | | | | protein 1 |
| Q9NRI6 | PYY2 | NM_021093 | 157195 | −0.28539523 | Peptide YY, 2 |
| | | | | | (seminalplasmin) |
| HSAC008290 | MGC5618 | BC016015 | 177781 | 0.28148678 | Hypothetical protein |
| | | | | | MGC5618 |
| O94927 | KIAA0841 | AB020648 | 7426 | −0.2681492 | KIAA0841 protein |
| PRL1_HUMAN | PROL1 | NM_021225 | 87198 | −0.26726353 | Proline-rich 1 |
| Q9NWQ8 | PAG | NM_018440 | 266175 | 0.26596376 | Phosphoprotein |
| | | | | | associated with |
| | | | | | glycosphingolipid- |
| | | | | | enriched microdomains |
| HSAC009739 | 0 | AK021991 | 296675 | 0.26376015 | Homo sapiens cDNA |
| | | | | | FLJ11929 fis, clone |
| | | | | | HEMBB1000434 |
| TAP4_HUMAN | TFAP4 | NM_003223 | 3005 | −0.24676795 | Transcription factor |
| | | | | | AP-4 (activating |
| | | | | | enhancer binding |
| | | | | | protein 4) |
| CSR3_HUMAN | CSRP3 | NM_003476 | 83577 | 0.22957104 | Cysteine and glycine- |
| | | | | | rich protein 3 (cardiac |
| | | | | | LIM protein) |
| MYH7_HUMAN | MYH7 | NM_000257 | 929 | 0.22417182 | Myosin, heavy |
| | | | | | polypeptide 7, cardiac |
| | | | | | muscle, beta |
| Q13513 | HSU47926 | NM_014262 | 46458 | 0.2822699 | Hypothetical protein B |
| CU97_HUMAN | FLJ21324 | AL161960 | 4746 | −0.27189524 | Hypothetical protein |
| | | | | | FLJ21324 |
| Q96EL2 | MRPS24 | NM_032014 | 284286 | 0.26906488 | Mitochondrial |
| | | | | | ribosomal protein S24 |
| PYR1_HUMAN | CAD | NM_004341 | 154868 | 0.26618776 | Carbamoyl-phosphate |
| | | | | | synthetase 2, aspartate |
| | | | | | transcarbamylase, and |
| | | | | | dihydroorotase |
| SPS2_HUMAN | SPS2 | NM_012248 | 118725 | 0.23578101 | Selenophosphate |
| | | | | | synthetase 2 |
| HSAC005553 | KIAA0483 | AB007952 | 64691 | −0.23107771 | KIAA0483 protein |
| Q8NC10 | FLJ20258 | NM_017729 | 28907 | −0.29044408 | Hypothetical protein |
| | | | | | FLJ20258 |
| HSAC007558 | 0 | AK024653 | 163440 | −0.28942837 | Homo sapiens cDNA: |
| | | | | | FLJ21000 fis, clone |
| | | | | | CAE03359 |
| Q96HT2 | 0 | BC008122 | 334931 | −0.28388928 | Homo sapiens, clone |
| | | | | | MGC: 18053 |
| | | | | | IMAGE: 4148889, |
| | | | | | mRNA, complete cds |
| P02570 | ACTB | NM_001101 | 288061 | 0.28095417 | Actin, beta |
| Q96HG1 | 0 | BC008642 | 42239 | −0.26271036 | Homo sapiens, clone |
| | | | | | IMAGE: 3868989, |
| | | | | | mRNA, partial cds |
| Q9H5C5 | FLJ23584 | NM_024588 | 22195 | −0.25933423 | Hypothetical protein |
| | | | | | FLJ23584 |
| HSAC014930 | 0 | AK022228 | 202577 | −0.25403533 | Homo sapiens cDNA |
| | | | | | FLJ12166 fis, clone |
| | | | | | MAMMA1000616 |
| Q8ND68 | PLVAP | NM_031310 | 107125 | 0.24973622 | Plasmalemma vesicle |
| | | | | | associated protein |
| Q9NPW0 | DKFZp547M236 | NM_018713 | 20981 | −0.24604121 | Hypothetical protein |
| | | | | | DKFZp547M236 |
| HSAC001572 | 0 | AK055659 | 8037 | 0.24451604 | Homo sapiens cDNA |
| | | | | | FLJ31097 fis, clone |
| | | | | | IMR321000210 |
| PARB_HUMAN | PARVB | NM_013327 | 8836 | −0.2429299 | Parvin beta |
| HSAC009747 | 0 | AK022065 | 296683 | −0.23257064 | Homo sapiens cDNA |
| | | | | | FLJ12003 fis, clone |
| | | | | | HEMBB1001537 |
| RL3_HUMAN | RPL3 | BC011860 | 119598 | 0.22612107 | Ribosomal protein L3 |
| CA1B_HUMAN | COL11A1 | NM_001854 | 82772 | 0.22373492 | Collagen, type XI, |
| | | | | | alpha 1 |
| STAT_HUMAN | STATH | NM_003154 | 37048 | −0.18441286 | Statherin |
| HSAC015960 | 0 | AF153502 | 247887 | 0.27007396 | Homo sapiens SNAI1P |
| | | | | | pseudogene |
| T1L1_HUMAN | TOM1L1 | NM_005486 | 153504 | −0.25974431 | Target of myb1-like 1 |
| | | | | | (chicken) |
| Q8WV53 | DKFZP564A2416 | NM_015535 | 5297 | 0.25296148 | DKFZP564A2416 |
| | | | | | protein |
| HS9A_HUMAN | HSPCA | AK056446 | 289088 | 0.25051774 | Heat shock 90 kD |
| | | | | | protein 1, alpha |
| BM88_HUMAN | BM88 | NM_016564 | 22140 | −0.24739798 | BM88 antigen |
| HSAC021209 | 0 | AK021479 | 351685 | −0.24183743 | Homo sapiens cDNA |
| | | | | | FLJ11417 fis, clone |
| | | | | | HEMBA1000960 |
| TIG2_HUMAN | RARRES2 | NM_002889 | 37682 | 0.24052315 | Retinoic acid receptor |
| | | | | | responder (tazarotene |
| | | | | | induced) 2 |
| ABC3_HUMAN | ABCA3 | NM_001089 | 26630 | −0.23736913 | ATP-binding cassette, |
| | | | | | sub-family A (ABC1), |
| | | | | | member 3 |
| Q96SG6 | 0 | BC014899 | 72087 | −0.23557118 | Homo sapiens, clone |
| | | | | | IMAGE: 3912307, |
| | | | | | mRNA |
| COP1_HUMAN | 0 | AK056926 | 350222 | 0.23440455 | Homo sapiens cDNA |
| | | | | | FLJ32364 fis, clone |
| | | | | | PUAEN1000147 |
| HSAC005034 | 0 | AF070602 | 51649 | −0.22614962 | Homo sapiens clone |
| | | | | | 24504 mRNA |
| | | | | | sequence |
| Q9BV20 | MGC3207 | AK026666 | 13075 | 0.21874807 | Hypothetical protein |
| | | | | | MGC3207 |
| IL8_HUMAN | IL8 | NM_000584 | 624 | 0.21295363 | Interleukin 8 |
| TGM3_HUMAN | TGM3 | NM_003245 | 2022 | −0.30559572 | Transglutaminase 3 (E |
| | | | | | polypeptide, protein- |
| | | | | | glutamine-gamma- |
| | | | | | glutamyltransferase) |
| FILA_HUMAN | 0 | BE551792 | 251440 | −0.2882993 | EST, Highly similar to |
| | | | | | A48118 major |
| | | | | | epidermal calcium- |
| | | | | | binding protein |
| | | | | | profilaggrin [H. sapiens] |
| Q96DP9 | 0 | AK055428 | 351007 | −0.28316024 | Homo sapiens cDNA |
| | | | | | FLJ30866 fis, clone |
| | | | | | FEBRA2004110, highly |
| | | | | | similar to |
| | | | | | PHOSPHOLIPASE |
| | | | | | ADRAB-B PRECURSO |
| Q9C0G8 | KIAA1695 | AB051482 | 288841 | 0.27341074 | Hypothetical protein |
| | | | | | FLJ22297 |
| PPIG_HUMAN | PPIG | NM_004792 | 77965 | −0.26853004 | Peptidyl-prolyl |
| | | | | | isomerase G |
| | | | | | (cyclophilin G) |
| ANX1_HUMAN | ANXA1 | NM_000700 | 78225 | −0.26467494 | Annexin A1 |
| Q9H8H3 | DKFZP586A0522 | NM_014033 | 288771 | −0.26179709 | DKFZP586A0522 |
| | | | | | protein |
| ENOA_HUMAN | ENO1 | NM_001428 | 254105 | 0.25723939 | Enolase 1, (alpha) |
| SOX5_HUMAN | SOX5 | NM_006940 | 87224 | −0.25633005 | SRY (sex determining |
| | | | | | region Y)-box 5 |
| MSX1_HUMAN | MSX1 | NM_002448 | 1494 | 0.2563254 | Msh homeo box |
| | | | | | homolog 1 (Drosophila) |
| HSAC019559 | 0 | AK021983 | 334535 | −0.25479699 | Homo sapiens cDNA |
| | | | | | FLJ11921 fis, clone |
| | | | | | HEMBB1000318 |
| HSAC016142 | 0 | X76785 | 249131 | 0.24660385 | H. sapiens genomic |
| | | | | | DNA, integration site |
| | | | | | for Epstein-Barr virus |
| Q9HBZ7 | FLJ11305 | AK024478 | 7049 | 0.24408926 | Hypothetical protein |
| | | | | | FLJ11305 |
| CMST_HUMAN | SLC35A1 | NM_006416 | 82921 | −0.23742472 | Solute carrier family 35 |
| | | | | | (CMP-sialic acid |
| | | | | | transporter), member 1 |
| SIM1_HUMAN | SIM1 | NM_005068 | 105925 | −0.23141341 | Single-minded |
| | | | | | homolog 1 (Drosophila) |
| Q8IXU2 | FLJ20190 | NM_017705 | 257511 | 0.21886983 | Hypothetical protein |
| | | | | | FLJ20190 |
| C166_HUMAN | ALCAM | Y10183 | 10247 | 0.19097406 | Activated leucocyte cell |
| | | | | | adhesion molecule |
| Q96AP0 | 24432 | NM_022914 | 78019 | 0.1866136 | Hypothetical protein |
| | | | | | 24432 |
| HSAC018926 | 0 | AK000989 | 319540 | −0.1726173 | Homo sapiens cDNA |
| | | | | | FLJ10127 fis, clone |
| | | | | | HEMBA1002973, |
| | | | | | moderately similar to |
| | | | | | CAMP-DEPENDENT |
| | | | | | 3′,5′-CYCLI |
| HSAC019510 | H2AFKP | Z80777 | 334456 | −0.2885027 | H2A histone family, |
| | | | | | member K, |
| | | | | | pseudogene |
| HSAC021283 | 0 | BC018033 | 351860 | −0.27842193 | Homo sapiens, clone |
| | | | | | IMAGE: 4800052, |
| | | | | | mRNA, partial cds |
| Q9NQ25 | CRACC | AB027233 | 132906 | −0.25159742 | 19A24 protein |
| MGP_HUMAN | MGP | NM_000900 | 279009 | 0.25075401 | Matrix Gla protein |
| HSAC018514 | 0 | AK024924 | 306729 | −0.24315744 | Homo sapiens cDNA: |
| | | | | | FLJ21271 fis, clone |
| | | | | | COL01751 |
| ABC6_HUMAN | ABCB6 | NM_005689 | 107911 | 0.24312557 | ATP-binding cassette, |
| | | | | | sub-family B |
| | | | | | (MDR/TAP), member 6 |
| HSAC016919 | HEP27 | NM_005794 | 272499 | 0.23808329 | Short-chain alcohol |
| | | | | | dehydrogenase family |
| | | | | | member |
| CD45_HUMAN | PTPRC | NM_002838 | 170121 | −0.23455219 | Protein tyrosine |
| | | | | | phosphatase, receptor |
| | | | | | type, C |
| Q9ULT2 | KIAA1138 | AB032964 | 115726 | −0.22789488 | KIAA1138 protein |
| SY3L_HUMAN | SCYA3 | NM_002983 | 73817 | 0.22252932 | Small inducible |
| | | | | | cytokine A3 |
| MM26_HUMAN | MMP26 | NM_021801 | 204732 | 0.18951291 | Matrix |
| | | | | | metalloproteinase 26 |
| PRL4_HUMAN | PROL4 | NM_007244 | 45033 | −0.01191859 | Proline rich 4 (lacrimal) |
| P78545 | ELF3 | NM_004433 | 166096 | −0.29828398 | E74-like factor 3 (ets |
| | | | | | domain transcription |
| | | | | | factor, epithelial- |
| | | | | | specific) |
| NIC1_HUMAN | NICE-1 | NM_019060 | 110196 | −0.2971944 | NICE-1 protein |
| CLD7_HUMAN | CLDN7 | NM_001307 | 278562 | −0.28206037 | Claudin 7 |
| KLKA_HUMAN | 0 | AK026045 | 275464 | −0.28180039 | Homo sapiens cDNA: |
| | | | | | FLJ22392 fis, clone |
| | | | | | HRC07868 |
| Q9P0A7 | C20orf30 | NM_014145 | 3576 | 0.27874372 | Chromosome 20 open |
| | | | | | reading frame 30 |
| HSAC019283 | 0 | AK026112 | 330418 | −0.27010085 | Homo sapiens cDNA: |
| | | | | | FLJ22459 fis, clone |
| | | | | | HRC10045 |
| Q9NYJ6 | LOC51328 | NM_016637 | 272413 | −0.2695095 | Ncaml |
| Q96HJ4 | NUF2R | NM_031423 | 234545 | −0.2663022 | Hypothetical protein |
| | | | | | NUF2R |
| PAB4_HUMAN | PABPC4 | NM_003819 | 169900 | 0.25652471 | Poly(A) binding protein, |
| | | | | | cytoplasmic 4 |
| | | | | | (inducible form) |
| HSAC012638 | FLJ12994 | NM_022841 | 126908 | −0.25215608 | Hypothetical protein |
| | | | | | FLJ12994 |
| HS47_HUMAN | SERPINH2 | NM_001235 | 9930 | 0.2460832 | Serine (or cysteine) |
| | | | | | proteinase inhibitor, |
| | | | | | clade H (heat shock |
| | | | | | protein 47), member 2 |
| Q9ULL9 | KIAA1201 | AB033027 | 251278 | −0.24435799 | KIAA1201 protein |
| TYY1_HUMAN | YY1 | NM_003403 | 97496 | 0.23844943 | YY1 transcription factor |
| HSAC016540 | ENPP3 | NM_005021 | 264750 | 0.23341437 | Ectonucleotide |
| | | | | | pyrophosphatase/phosphodiesterase 3 |
| IP3K_HUMAN | ITPKA | NM_002220 | 2722 | 0.231694 | Inositol 1,4,5- |
| | | | | | trisphosphate 3-kinase A |
| HSAC009930 | MGC17528 | BC010541 | 300893 | −0.23039734 | Hypothetical protein |
| | | | | | MGC17528 |
| CRE3_HUMAN | CREB3 | NM_006368 | 287921 | 0.22584512 | CAMP responsive |
| | | | | | element binding protein |
| | | | | | 3 (luman) |
| HSAC016682 | 0 | AK057367 | 270002 | −0.22515872 | Homo sapiens cDNA |
| | | | | | FLJ32805 fis, clone |
| | | | | | TESTI2002690 |
| TFE2_HUMAN | TCF3 | M31523 | 101047 | 0.21777199 | Transcription factor 3 |
| | | | | | (E2A immunoglobulin |
| | | | | | enhancer binding |
| | | | | | factors E12/E47) |
| BIN1_HUMAN | BIN1 | NM_004305 | 193163 | 0.2155171 | Bridging integrator 1 |
| HSAC012959 | 0 | AK023520 | 131798 | −0.21368949 | Homo sapiens cDNA |
| | | | | | FLJ13458 fis, clone |
| | | | | | PLACE1003361 |
| Q64320 | STXBP1 | NM_003165 | 239356 | −0.19357344 | Syntaxin binding |
| | | | | | protein 1 |
| 3BH1_HUMAN | HSD3B1 | NM_000862 | 38586 | −0.19079495 | Hydroxy-delta-5-steroid |
| | | | | | dehydrogenase, 3 |
| | | | | | beta- and steroid delta- |
| | | | | | isomerase 1 |
| P04270 | ACTA1 | NM_001100 | 1288 | 0.17079596 | Actin, alpha 1, skeletal |
| | | | | | muscle |
| GLPB_HUMAN | GYPA | NM_002099 | 108694 | −0.2797532 | Glycophorin A |
| | | | | | (includes MN blood |
| | | | | | group) |
| TIG3_HUMAN | RARRES3 | NM_004585 | 17466 | −0.26355336 | Retinoic acid receptor |
| | | | | | responder (tazarotene |
| | | | | | induced) 3 |
| Q9HAE0 | FLJ11783 | NM_024891 | 232167 | −0.26191261 | Hypothetical protein |
| | | | | | FLJ11783 |
| OFU1_HUMAN | POFUT1 | AF375884 | 178292 | 0.25365544 | Protein O- |
| | | | | | fucosyltransferase 1 |
| HSAC009500 | MMPL1 | NM_004142 | 290222 | −0.25346089 | Matrix |
| | | | | | metalloproteinase-like 1 |
| COXK_HUMAN | COX7A1 | NM_001864 | 114346 | 0.25190367 | Cytochrome c oxidase |
| | | | | | subunit VIIa |
| | | | | | polypeptide 1 (muscle) |
| BTE1_HUMAN | BTEB1 | NM_001206 | 150557 | 0.24546012 | Basic transcription |
| | | | | | element binding protein |
| OPSD_HUMAN | RHO | NM_000539 | 247565 | 0.24293319 | Rhodopsin (opsin 2, |
| | | | | | rod pigment) (retinitis |
| | | | | | pigmentosa 4, |
| | | | | | autosomal dominant) |
| PLCB_HUMAN | AGPAT2 | NM_006412 | 209119 | 0.24123053 | 1-acylglycerol-3- |
| | | | | | phosphate O- |
| | | | | | acyltransferase 2 |
| | | | | | (lysophosphatidic acid |
| | | | | | acyltransferase, beta) |
| Q15668 | NPC2 | NM_006432 | 119529 | 0.22752332 | Niemann-Pick disease, |
| | | | | | type C2 |
| Q9UBI4 | STOML1 | NM_004809 | 194816 | 0.21956228 | Stomatin (EBP72)-like 1 |
| AAP04413 | 0 | AL133654 | 201603 | 0.21881691 | Homo sapiens mRNA; |
| | | | | | cDNA |
| | | | | | DKFZp434D0917 (from |
| | | | | | clone |
| | | | | | DKFZp434D0917) |
| Q8WWF8 | 0 | BC017586 | 55150 | −0.19756688 | Homo sapiens, Similar |
| | | | | | to RIKEN cDNA |
| | | | | | 1700028N11 gene, |
| | | | | | clone MGC: 26610 |
| | | | | | IMAGE: 4837506, |
| | | | | | mRNA, complete c |
| HSAC018492 | 0 | AK024800 | 306704 | −0.28750526 | Homo sapiens cDNA: |
| | | | | | FLJ21147 fis, clone |
| | | | | | CAS09371 |
| Q9C0D8 | 0 | BC008941 | 207024 | −0.28684225 | Homo sapiens, Similar |
| | | | | | to hypothetical protein |
| | | | | | FLJ20515, clone |
| | | | | | MGC: 2696 |
| | | | | | IMAGE: 2820596, |
| | | | | | mRNA, complete |
| DDC_HUMAN | DDC | NM_000790 | 150403 | −0.2843375 | Dopa decarboxylase |
| | | | | | (aromatic L-amino acid |
| | | | | | decarboxylase) |
| FBW4_HUMAN | SHFM3 | AK056917 | 24307 | 0.28026937 | Split hand/foot |
| | | | | | malformation |
| | | | | | (ectrodactyly) type 3 |
| QCAA000051 | 0 | 0 | 0 | −0.26836294 | Randomized negative |
| | | | | | control |
| Q9UM77 | OR1E3P | U53583 | 248182 | −0.2620377 | Olfactory receptor, |
| | | | | | family 1, subfamily E, |
| | | | | | member 3 pseudogene |
| TYBP_HUMAN | TYROBP | NM_003332 | 9963 | −0.25688511 | TYRO protein tyrosine |
| | | | | | kinase binding protein |
| SCP1_HUMAN | SYCP1 | NM_003176 | 112743 | −0.25216102 | Synaptonemal complex |
| | | | | | protein 1 |
| Q9HCH7 | KIAA1595 | AB046815 | 286173 | 0.25125685 | KIAA1595 protein |
| HSAC012142 | ERH | NM_004450 | 118757 | 0.24637661 | Enhancer of |
| | | | | | rudimentary homolog |
| | | | | | (Drosophila) |
| ACLY_HUMAN | ACLY | NM_001096 | 174140 | 0.24619185 | ATP citrate lyase |
| S3B2_HUMAN | SF3B2 | NM_006842 | 75916 | 0.24414637 | Splicing factor 3b, |
| | | | | | subunit 2, 145 kD |
| O14731 | PKMYT1 | NM_004203 | 77783 | 0.24202938 | Membrane-associated |
| | | | | | tyrosine- and |
| | | | | | threonine-specific |
| | | | | | cdc2-inhibitory kinase |
| HSAC003854 | 0 | AK025794 | 28631 | −0.24081021 | Homo sapiens cDNA: |
| | | | | | FLJ22141 fis, clone |
| | | | | | HEP21327 |
| HSAC020709 | 0 | AK057203 | 350710 | −0.23982763 | Homo sapiens cDNA |
| | | | | | FLJ32641 fis, clone |
| | | | | | SYNOV2001035 |
| UNRI_HUMAN | UNRIP | NM_007178 | 3727 | 0.23894091 | Unr-interacting protein |
| PGTA_HUMAN | RABGGTA | NM_004581 | 78920 | −0.23645356 | Rab |
| | | | | | geranylgeranyltransferase, |
| | | | | | alpha subunit |
| HSAC019191 | 0 | AL080233 | 326580 | −0.23022427 | Homo sapiens mRNA; |
| | | | | | cDNA DKFZp586L111 |
| | | | | | (from clone |
| | | | | | DKFZp586L111) |
| SDB2_HUMAN | SDCBP2 | NM_015685 | 64179 | −0.22859025 | Syndecan binding |
| | | | | | protein (syntenin) 2 |
| CTC9_HUMAN | C20orf129 | AK055793 | 70704 | −0.22775703 | Chromosome 20 open |
| | | | | | reading frame 129 |
| PCN2_HUMAN | PCNT2 | NM_006031 | 15896 | −0.22667113 | Pericentrin 2 (kendrin) |
| IF42_HUMAN | EIF4A2 | NM_001967 | 173912 | 0.22657769 | Eukaryotic translation |
| | | | | | initiation factor 4A, |
| | | | | | isoform 2 |
| HSAC018456 | 0 | AK024123 | 306664 | −0.22504349 | Homo sapiens cDNA |
| | | | | | FLJ14061 fis, clone |
| | | | | | HEMBB1000749 |
| HSAC018632 | 0 | AK026722 | 306870 | 0.22009923 | Homo sapiens cDNA: |
| | | | | | FLJ23069 fis, clone |
| | | | | | LNG05603 |
| ATS1_HUMAN | ADAMTS1 | NM_006988 | 8230 | 0.20713748 | A disintegrin-like and |
| | | | | | metalloprotease |
| | | | | | (reprolysin type) with |
| | | | | | thrombospondin type 1 |
| | | | | | motif, 1 |
| Q96DP3 | WAC | AB058747 | 70333 | 0.20422375 | WW domain-containing |
| | | | | | adapter with a coiled- |
| | | | | | coil region |
| HSAC002790 | DKFZp667O2416 | AK056427 | 19066 | −0.19264477 | Hypothetical protein |
| | | | | | DKFZp667O2416 |
| AN11_HUMAN | ANAPC11 | NM_016476 | 183180 | 0.18856896 | APC11 anaphase |
| | | | | | promoting complex |
| | | | | | subunit 11 homolog |
| | | | | | (yeast) |
| Q96M94 | KIAA1677 | AB051464 | 61603 | 0.17363087 | KIAA1677 |
| HSAC017386 | HSPC073 | NM_014163 | 278948 | 0.15730714 | HSPC073 protein |
| HSAC006154 | CD14 | NM_000591 | 75627 | 0.27102209 | CD14 antigen |
| PPIF_HUMAN | PPIF | NM_005729 | 173125 | −0.26443857 | Peptidylprolyl |
| | | | | | isomerase F |
| | | | | | (cyclophilin F) |
| Q9NT21 | 0 | AL137581 | 112589 | −0.26076922 | Homo sapiens mRNA; |
| | | | | | cDNA |
| | | | | | DKFZp434B0610 (from |
| | | | | | clone |
| | | | | | DKFZp434B0610); |
| | | | | | partial cds |
| Q9H6B9 | FLJ22408 | NM_024794 | 156457 | −0.25752275 | Hypothetical protein |
| | | | | | FLJ22408 |
| LDHA_HUMAN | LDHA | NM_005566 | 2795 | 0.25289918 | Lactate dehydrogenase A |
| H2AY_HUMAN | H2AFY | NM_004893 | 75258 | −0.2518442 | H2A histone family, |
| | | | | | member Y |
| HSAC009983 | 0 | AK022320 | 301232 | −0.2470859 | Homo sapiens cDNA |
| | | | | | FLJ12258 fis, clone |
| | | | | | MAMMA1001510 |
| HSAC014390 | 0 | AK001865 | 188228 | 0.24557304 | Homo sapiens cDNA |
| | | | | | FLJ11003 fis, clone |
| | | | | | PLACE1002851 |
| GT4R_HUMAN | SLC2A4RG | NM_020062 | 170088 | 0.24473836 | SLC2A4 regulator |
| FOS_HUMAN | FOS | NM_005252 | 25647 | −0.24335376 | V-fos FBJ murine |
| | | | | | osteosarcoma viral |
| | | | | | oncogene homolog |
| Q9BWL3 | DKFZP586G1722 | NM_015449 | 31989 | 0.24290984 | DKFZP586G1722 |
| | | | | | protein |
| CY1_HUMAN | CYC1 | NM_001916 | 289271 | 0.242676 | Cytochrome c-1 |
| Q8IX90 | 0 | BC013418 | 88523 | 0.24027868 | Homo sapiens, clone |
| | | | | | MGC: 4832 |
| | | | | | IMAGE: 3604003, |
| | | | | | mRNA, complete cds |
| GALA_HUMAN | GAL | M77140 | 1907 | 0.23487288 | Galanin |
| P02593 | CALM2 | AK055130 | 182278 | 0.23450179 | Calmodulin 2 |
| | | | | | (phosphorylase kinase, |
| | | | | | delta) |
| HSAC020657 | 0 | AK057533 | 350657 | 0.2332272 | Homo sapiens cDNA |
| | | | | | FLJ32971 fis, clone |
| | | | | | TESTI2008847 |
| Q9H125 | C20orf118 | AL079335 | 287784 | 0.22896758 | Chromosome 20 open |
| | | | | | reading frame 118 |
| Q8N8N8 | DKFZP434G145 | BC007359 | 31931 | 0.22106691 | DKFZP434G145 |
| | | | | | protein |
| GBAP_HUMAN | GABARAP | NM_007278 | 7719 | −0.22052485 | GABA(A) receptor- |
| | | | | | associated protein |
| DYI4_HUMAN | DNAI2 | NM_023036 | 147472 | 0.2163783 | Dynein intermediate |
| | | | | | chain 2 |
| HSAC017486 | HDAC3 | AF130111 | 279789 | −0.21279266 | Histone deacetylase 3 |
| NUBM_HUMAN | 0 | AK055875 | 324151 | 0.21264326 | Homo sapiens cDNA |
| | | | | | FLJ31313 fis, clone |
| | | | | | LIVER1000230, highly |
| | | | | | similar to NADH- |
| | | | | | UBIQUINONE |
| | | | | | OXIDOREDUCTASE |
| NRCA_HUMAN | NRCAM | NM_005010 | 7912 | −0.20902953 | Neuronal cell adhesion |
| | | | | | molecule |
| IF42_HUMAN | EIF4A2 | NM_001967 | 173912 | 0.20860474 | Eukaryotic translation |
| | | | | | initiation factor 4A, |
| | | | | | isoform 2 |
| CATB_HUMAN | CTSB | NM_001908 | 297939 | 0.20693544 | Cathepsin B |
| HSAC009142 | 0 | AK026499 | 287713 | −0.20623884 | Homo sapiens cDNA: |
| | | | | | FLJ22752 fis, clone |
| | | | | | KAIA0555 |
| NGAP_HUMAN | RASAL2 | NM_004841 | 227806 | 0.18773486 | RAS protein activator |
| | | | | | like 2 |
| H10_HUMAN | H1F0 | BC000145 | 226117 | 0.18021445 | H1 histone family, |
| | | | | | member 0 |
| RNP_HUMAN | RNASE1 | NM_002933 | 78224 | 0.17681221 | Ribonuclease, RNase |
| | | | | | A family, 1 (pancreatic) |
| HSAC021160 | 0 | AK054984 | 351562 | 0.13703193 | Homo sapiens cDNA |
| | | | | | FLJ30422 fis, clone |
| | | | | | BRACE2008861 |
| Q9BYE3 | LEP16 | NM_032563 | 244349 | −0.28446955 | Epidermal |
| | | | | | differentiation complex |
| | | | | | protein like protein |
| O75042 | 0 | AK024906 | 160613 | −0.26547427 | Homo sapiens cDNA: |
| | | | | | FLJ21253 fis, clone |
| | | | | | COL01316 |
| HSAC017211 | 0 | AK021887 | 277001 | −0.26476404 | Homo sapiens cDNA |
| | | | | | FLJ11825 fis, clone |
| | | | | | HEMBA1006494 |
| IDI1_HUMAN | IDI1 | NM_004508 | 76038 | −0.25999498 | Isopentenyl- |
| | | | | | diphosphate delta |
| | | | | | isomerase |
| HSAC006238 | TMSB4X | AK055976 | 75968 | 0.25632509 | Thymosin, beta 4, X |
| | | | | | chromosome |
| ETS2_HUMAN | ETS2 | NM_005239 | 85146 | −0.2516845 | V-ets erythroblastosis |
| | | | | | virus E26 oncogene |
| | | | | | homolog 2 (avian) |
| SSB4_HUMAN | MGC3181 | NM_032627 | 324618 | 0.24973691 | Hypothetical protein |
| | | | | | MGC3181 |
| O60426 | FADS3 | NM_021727 | 21765 | 0.24927285 | Fatty acid desaturase 3 |
| LDHA_HUMAN | LDHA | NM_005566 | 2795 | 0.24753928 | Lactate dehydrogenase A |
| KB15_HUMAN | KIAA1115 | NM_014931 | 72172 | −0.24331719 | KIAA1115 protein |
| HSAC014795 | PLSCR1 | NM_021105 | 198282 | −0.24294281 | Phospholipid |
| | | | | | scramblase 1 |
| CO1A_HUMAN | CORO1A | NM_007074 | 109606 | −0.24222576 | Coronin, actin binding |
| | | | | | protein, 1A |
| K1CP_HUMAN | KRT16 | NM_005557 | 115947 | −0.23828098 | Keratin 16 (focal non- |
| | | | | | epidermolytic |
| | | | | | palmoplantar |
| | | | | | keratoderma) |
| CT11_HUMAN | C20orf11 | NM_017896 | 103808 | 0.23722689 | Chromosome 20 open |
| | | | | | reading frame 11 |
| HSAC016239 | 0 | AL049369 | 250724 | −0.23637937 | Homo sapiens mRNA; |
| | | | | | cDNA |
| | | | | | DKFZp586D0518 (from |
| | | | | | clone |
| | | | | | DKFZp586D0518) |
| LDHA_HUMAN | LDHA | NM_005566 | 2795 | 0.23548608 | Lactate dehydrogenase A |
| ST24_HUMAN | STK24 | NM_003576 | 168913 | −0.23478567 | Serine/threonine |
| | | | | | kinase 24 (STE20 |
| | | | | | homolog, yeast) |
| O60527 | SDCCAG8 | AF039690 | 300642 | −0.23195532 | Serologically defined |
| | | | | | colon cancer antigen 8 |
| HSAC018030 | 0 | AL049275 | 302051 | −0.23020458 | Homo sapiens mRNA; |
| | | | | | cDNA DKFZp564H213 |
| | | | | | (from clone |
| | | | | | DKFZp564H213) |
| HSAC018565 | 0 | AK025194 | 306784 | 0.22999594 | Homo sapiens cDNA: |
| | | | | | FLJ21541 fis, clone |
| | | | | | COL06166 |
| PO43_HUMAN | POU4F3 | NM_002700 | 248019 | −0.22881509 | POU domain, class 4, |
| | | | | | transcription factor 3 |
| HSAC015174 | 0 | AC006328 | 213956 | 0.22863598 | Homo sapiens BAC |
| | | | | | clone RP11-102O5 |
| | | | | | from Y |
| Q9H0N3 | DKFZP566M1046 | NM_032127 | 8039 | 0.22863435 | Hypothetical protein |
| | | | | | DKFZp566M1046 |
| 7B2_HUMAN | SGNE1 | NM_003020 | 2265 | −0.22641912 | Secretory granule, |
| | | | | | neuroendocrine protein |
| | | | | | 1 (7B2 protein) |
| IP3S_HUMAN | ITPR2 | NM_002223 | 238272 | −0.2261709 | Inositol 1,4,5- |
| | | | | | triphosphate receptor, |
| | | | | | type 2 |
| Q9UEF2 | UBC | M26880 | 183704 | −0.22356958 | Ubiquitin C |
| HSAC018590 | 0 | AK025451 | 306812 | −0.22302005 | Homo sapiens cDNA: |
| | | | | | FLJ21798 fis, clone |
| | | | | | HEP00573 |
| Q8WY82 | 0 | BC006284 | 333388 | −0.22271485 | Homo sapiens, clone |
| | | | | | IMAGE: 3957135, |
| | | | | | mRNA, partial cds |
| TSP4_HUMAN | THBS4 | NM_003248 | 75774 | 0.22139735 | Thrombospondin 4 |
| HSAC002075 | LOC51170 | NM_016245 | 12150 | 0.22091464 | Retinal short-chain |
| | | | | | dehydrogenase/reductase |
| | | | | | retSDR2 |
| HSAC009969 | 0 | AK021935 | 301153 | 0.21899011 | Homo sapiens cDNA |
| | | | | | FLJ11873 fis, clone |
| | | | | | HEMBA1007066 |
| HSAC007149 | 0 | AL080078 | 85335 | −0.21515156 | Homo sapiens mRNA; |
| | | | | | cDNA |
| | | | | | DKFZp564D1462 (from |
| | | | | | clone |
| | | | | | DKFZp564D1462) |
| MK01_HUMAN | MAPK1 | AL157438 | 324473 | 0.21202447 | Mitogen-activated |
| | | | | | protein kinase 1 |
| 9KD_HUMAN | MGC10471 | NM_030818 | 24998 | −0.21138421 | Hypothetical protein |
| | | | | | MGC10471 |
| HSAC018712 | KAP4.14 | NM_033059 | 307015 | −0.21048412 | Keratin associated |
| | | | | | protein 4.14 |
| TYPH_HUMAN | ECGF1 | NM_001953 | 73946 | −0.20356611 | Endothelial cell growth |
| | | | | | factor 1 (platelet- |
| | | | | | derived) |
| Q9UBF4 | KIAA1036 | NM_014909 | 155182 | 0.20323707 | KIAA1036 protein |
| CIQ5_HUMAN | KCNQ5 | NM_019842 | 283644 | 0.20086104 | Potassium voltage- |
| | | | | | gated channel, KQT- |
| | | | | | like subfamily, member 5 |
| P39028 | RPS23 | NM_001025 | 3463 | 0.19967718 | Ribosomal protein S23 |
| NF3L_HUMAN | NIF3L1 | NM_021824 | 21943 | −0.18719016 | NIF3 NGG1 interacting |
| | | | | | factor 3-like 1 (S. pombe) |
| GTO1_HUMAN | GSTTLp28 | NM_004832 | 11465 | 0.17999128 | Glutathione-S- |
| | | | | | transferase like; |
| | | | | | glutathione transferase |
| | | | | | omega |
| RAC2_HUMAN | HSPC022 | NM_014029 | 301175 | 0.17873117 | HSPC022 protein |
| Q9HAC8 | FLJ11807 | NM_024954 | 285813 | 0.17686817 | Hypothetical protein |
| | | | | | FLJ11807 |
| RALA_HUMAN | 0 | AK026850 | 6906 | 0.17343054 | Homo sapiens cDNA: |
| | | | | | FLJ23197 fis, clone |
| | | | | | REC00917 |
| CSR2_HUMAN | CSRP2 | NM_001321 | 10526 | 0.17334591 | Cysteine and glycine- |
| | | | | | rich protein 2 |
| HSAC019311 | 0 | BC005220 | 332008 | −0.17218654 | Homo sapiens, Similar |
| | | | | | to chaperonin |
| | | | | | containing TCP1, |
| | | | | | subunit 8 (theta), clone |
| | | | | | MGC: 12240 |
| | | | | | IMAGE: 393033 |
| MNK2_HUMAN | GPRK7 | NM_017572 | 261828 | −0.16694331 | G protein-coupled |
| | | | | | receptor kinase 7 |
| HSAC014448 | FLJ13385 | NM_024853 | 190279 | −0.16552904 | Hypothetical protein |
| | | | | | FLJ13385 |
| 143F_HUMAN | YWHAH | NM_003405 | 349530 | 0.14759809 | Tyrosine 3- |
| | | | | | monooxygenase/tryptophan |
| | | | | | 5- |
| | | | | | monooxygenase |
| | | | | | activation protein, eta |
| | | | | | polypeptide |
| Q96MZ7 | 0 | AK056203 | 323813 | 0.1170698 | Homo sapiens, clone |
| | | | | | MGC: 2867 |
| | | | | | IMAGE: 2988664, |
| | | | | | mRNA, complete cds |
| LOXR_HUMAN | ALOX12B | NM_001139 | 136574 | −0.33086759 | Arachidonate 12- |
| | | | | | lipoxygenase, 12R type |
| BD02_HUMAN | DEFB2 | NM_004942 | 105924 | −0.30510738 | Defensin, beta 2 |
| HSAC016948 | FLJ20433 | NM_017820 | 272799 | −0.29024904 | Hypothetical protein |
| | | | | | FLJ20433 |
| S112_HUMAN | S100A12 | NM_005621 | 19413 | −0.28725669 | S100 calcium binding |
| | | | | | protein A12 |
| | | | | | (calgranulin C) |
| HSAC018084 | 0 | AF130062 | 302146 | −0.25929293 | Homo sapiens clone |
| | | | | | FLB7715 PRO2051 |
| | | | | | mRNA, complete cds |
| Q9NPA8 | DC6 | NM_020189 | 283740 | 0.25384995 | DC6 protein |
| HSAC004275 | 0 | AK001638 | 34790 | −0.24604722 | Homo sapiens cDNA |
| | | | | | FLJ10776 fis, clone |
| | | | | | NT2RP4000323 |
| HSAC018560 | 0 | AK025177 | 306778 | −0.24446302 | Homo sapiens cDNA: |
| | | | | | FLJ21524 fis, clone |
| | | | | | COL05921 |
| HSAC020812 | 0 | AK055957 | 350815 | −0.24373933 | Homo sapiens cDNA |
| | | | | | FLJ31395 fis, clone |
| | | | | | NT2NE1000122 |
| HSAC018421 | 0 | AK022022 | 306620 | −0.24043929 | Homo sapiens cDNA |
| | | | | | FLJ11960 fis, clone |
| | | | | | HEMBB1001008 |
| O95204 | MP1 | NM_014889 | 260116 | −0.23934663 | Metalloprotease 1 |
| | | | | | (pitrilysin family) |
| Q96IC1 | AGRN | AF016903 | 273330 | 0.23808476 | Agrin |
| AKBA_HUMAN | AKR1B10 | NM_020299 | 116724 | −0.23802389 | Aldo-keto reductase |
| | | | | | family 1, member B10 |
| | | | | | (aldose reductase) |
| LDHA_HUMAN | LDHA | NM_005566 | 2795 | 0.23795266 | Lactate dehydrogenase A |
| PTK6_HUMAN | PTK6 | NM_005975 | 51133 | −0.23581278 | PTK6 protein tyrosine |
| | | | | | kinase 6 |
| NUOM_HUMAN | NDUFV3 | NM_021075 | 351217 | −0.23366493 | NADH dehydrogenase |
| | | | | | (ubiquinone) |
| | | | | | flavoprotein 3 (10 kD) |
| GBP2_HUMAN | GBP2 | NM_004120 | 171862 | −0.23313126 | Guanylate binding |
| | | | | | protein 2, interferon- |
| | | | | | inducible |
| Q9Y2I3 | KIAA0977 | NM_014900 | 300855 | −0.23204872 | KIAA0977 protein |
| ATS2_HUMAN | ADAMTS2 | NM_014244 | 120330 | 0.2317392 | A disintegrin-like and |
| | | | | | metalloprotease |
| | | | | | (reprolysin type) with |
| | | | | | thrombospondin type 1 |
| | | | | | motif, 2 |
| Q9P1G3 | PRO1853 | NM_018607 | 52891 | −0.23061723 | Hypothetical protein |
| | | | | | PRO1853 |
| Q92616 | GCN1L1 | D86973 | 75354 | 0.22882625 | GCN1 general control |
| | | | | | of amino-acid synthesis |
| | | | | | 1-like 1 (yeast) |
| SLI1_HUMAN | FHL1 | NM_001449 | 239069 | 0.22601495 | Four and a half LIM |
| | | | | | domains 1 |
| HSAC010091 | R33729_1 | Z78330 | 10927 | −0.22537096 | Hypothetical protein |
| | | | | | R33729_1 |
| S6AC_HUMAN | SLC6A12 | U27699 | 82535 | −0.22507903 | Solute carrier family 6 |
| | | | | | (neurotransmitter |
| | | | | | transporter, |
| | | | | | betaine/GABA), |
| | | | | | member 12 |
| Y110_HUMAN | KIAA0110 | NM_014628 | 124 | 0.22461245 | Gene predicted from |
| | | | | | cDNA with a complete |
| | | | | | coding sequence |
| HSAC013952 | SP3 | X68560 | 154295 | −0.22445276 | Sp3 transcription factor |
| SYD_HUMAN | DARS | NM_001349 | 80758 | 0.22434629 | Aspartyl-tRNA |
| | | | | | synthetase |
| RL7_HUMAN | RPL7 | NM_000971 | 153 | 0.22410736 | Ribosomal protein L7 |
| SNK_HUMAN | SNK | NM_006622 | 3838 | −0.22350301 | Serum-inducible kinase |
| ID3_HUMAN | ID3 | NM_002167 | 76884 | −0.22236535 | Inhibitor of DNA |
| | | | | | binding 3, dominant |
| | | | | | negative helix-loop- |
| | | | | | helix protein |
| RMP1_HUMAN | RAMP1 | NM_005855 | 32989 | 0.22176553 | Receptor (calcitonin) |
| | | | | | activity modifying |
| | | | | | protein 1 |
| FK26_HUMAN | KIAA1049 | NM_014972 | 227835 | 0.22124994 | KIAA1049 protein |
| IEX1_HUMAN | IER3 | NM_052815 | 76095 | 0.22091401 | Immediate early |
| | | | | | response 3 |
| PTPK_HUMAN | PTPRK | NM_002844 | 79005 | 0.22006385 | Protein tyrosine |
| | | | | | phosphatase, receptor |
| | | | | | type, K |
| HPS4_HUMAN | 0 | AK057648 | 350640 | 0.21953415 | Homo sapiens cDNA |
| | | | | | FLJ33086 fis, clone |
| | | | | | TRACH2000461 |
| LDHB_HUMAN | LDHB | BC008952 | 234489 | 0.21684934 | Lactate dehydrogenase B |
| Q8NDB6 | PRO0659 | AK054721 | 6451 | 0.21637177 | PRO0659 protein |
| N7BM_HUMAN | DAP13 | NM_018838 | 44163 | −0.21579024 | 13 kDa differentiation- |
| | | | | | associated protein |
| Q9H7Z7 | C9orf15 | NM_025072 | 288102 | −0.21525058 | Chromosome 9 open |
| | | | | | reading frame 15 |
| TM22_HUMAN | TRIM22 | NM_006074 | 318501 | −0.21520167 | Tripartite motif- |
| | | | | | containing 22 |
| Q9H749 | DKFZP586D0919 | AL050100 | 49378 | 0.21169944 | DKFZP586D0919 |
| | | | | | protein |
| Q969K7 | CAC-1 | NM_033504 | 343912 | −0.21091675 | CAC-1 |
| OAS3_HUMAN | OAS3 | NM_006187 | 56009 | −0.20966638 | 2′-5′-oligoadenylate |
| | | | | | synthetase 3 (100 kD) |
| PAN2_HUMAN | PANX2 | NM_052839 | 343259 | 0.20929628 | Pannexin 2 |
| IFT1_HUMAN | IFIT1 | NM_001548 | 20315 | −0.20924493 | Interferon-induced |
| | | | | | protein with |
| | | | | | tetratricopeptide |
| | | | | | repeats 1 |
| Q96HK5 | LOC51030 | NM_016078 | 6776 | −0.20809385 | CGI-148 protein |
| DDX3_HUMAN | DDX3 | NM_001356 | 147916 | −0.20803591 | DEAD/H (Asp-Glu-Ala- |
| | | | | | Asp/His) box |
| | | | | | polypeptide 3 |
| IF42_HUMAN | EIF4A2 | NM_001967 | 173912 | 0.20756638 | Eukaryotic translation |
| | | | | | initiation factor 4A, |
| | | | | | isoform 2 |
| Q9NPI0 | 0 | AK027724 | 334557 | −0.20655815 | Homo sapiens cDNA |
| | | | | | FLJ14818 fis, clone |
| | | | | | OVARC1000168 |
| P11082 | PPP2CB | NM_004156 | 80350 | −0.20633072 | Protein phosphatase 2 |
| | | | | | (formerly 2A), catalytic |
| | | | | | subunit, beta isoform |
| HSAC004729 | 0 | AK021604 | 44787 | −0.20569729 | Homo sapiens mRNA; |
| | | | | | cDNA |
| | | | | | DKFZp434O0227 (from |
| | | | | | clone |
| | | | | | DKFZp434O0227) |
| THTR_HUMAN | TST | NM_003312 | 351863 | −0.20339936 | Thiosulfate |
| | | | | | sulfurtransferase |
| | | | | | (rhodanese) |
| APL1_HUMAN | APOL1 | AF323540 | 114309 | −0.2014645 | Apolipoprotein L, 1 |
| Q96IP9 | H41 | AF103803 | 283690 | −0.19892848 | Hypothetical protein |
| PSB5_HUMAN | PSMB5 | NM_002797 | 78596 | 0.19884452 | Proteasome (prosome, |
| | | | | | macropain) subunit, |
| | | | | | beta type, 5 |
| Q96MH6 | 0 | AK056932 | 280858 | −0.19855951 | Homo sapiens cDNA |
| | | | | | FLJ32370 fis, clone |
| | | | | | PUAEN1000322 |
| Q96DR8 | LOC118430 | NM_058173 | 348419 | −0.19728271 | Small breast epithelial |
| | | | | | mucin |
| HSAC020134 | 0 | BG912466 | 347715 | −0.19659403 | Homo sapiens mRNA |
| | | | | | full length insert cDNA |
| | | | | | clone EUROIMAGE |
| | | | | | 152428 |
| APA1_HUMAN | APOA1 | NM_000039 | 93194 | 0.19456431 | Apolipoprotein A-I |
| IKBA_HUMAN | NFKBIA | NM_020529 | 81328 | 0.19434039 | Nuclear factor of kappa |
| | | | | | light polypeptide gene |
| | | | | | enhancer in B-cells |
| | | | | | inhibitor, alpha |
| S61G_HUMAN | SEC61G | NM_014302 | 9950 | 0.19385443 | Sec61 gamma |
| Q8TB66 | NIFK | NM_032390 | 142838 | 0.19235852 | Nucleolar protein |
| | | | | | interacting with the |
| | | | | | FHA domain of pKi-67 |
| FXLA_HUMAN | PCCX2 | AB031230 | 199009 | −0.19227016 | Protein containing |
| | | | | | CXXC domain 2 |
| HSAC005389 | PRO2086 | NM_014111 | 60082 | −0.19183233 | PRO2086 protein |
| TGDS_HUMAN | TDPGD | NM_014305 | 12393 | 0.19120788 | DTDP-D-glucose 4,6- |
| | | | | | dehydratase |
| Q9NYL1 | PTOV1 | NM_017432 | 19555 | 0.18942731 | Prostate tumor over |
| | | | | | expressed gene 1 |
| RET7_HUMAN | RBP7 | NM_052960 | 292718 | 0.18902609 | Retinoid binding |
| | | | | | protein 7 |
| Q9H5V9 | FLJ22965 | NM_022101 | 248572 | 0.18655842 | Hypothetical protein |
| | | | | | FLJ22965 |
| O94911 | ABCA8 | NM_007168 | 38095 | −0.18602829 | ATP-binding cassette, |
| | | | | | sub-family A (ABC1), |
| | | | | | member 8 |
| AAH50307 | MORC | NM_014429 | 278908 | −0.18567819 | Microrchidia homolog |
| | | | | | (mouse) |
| TR1B_HUMAN | TNFRSF1B | NM_001066 | 256278 | 0.18560882 | Tumor necrosis factor |
| | | | | | receptor superfamily, |
| | | | | | member 1B |
| HS71_HUMAN | HSPA1A | NM_005345 | 8997 | 0.18329446 | Heat shock 70 kD |
| | | | | | protein 1A |
| Q9C086 | PAPA-1 | NM_031288 | 118282 | −0.18173371 | PAP-1 binding protein |
| HSAC005625 | 0 | AL050367 | 66762 | 0.17583034 | Homo sapiens mRNA; |
| | | | | | cDNA DKFZp564A026 |
| | | | | | (from clone |
| | | | | | DKFZp564A026) |
| HSAC000871 | 0 | AK024270 | 4094 | 0.17415618 | Homo sapiens cDNA |
| | | | | | FLJ14208 fis, clone |
| | | | | | NT2RP3003264 |
| Q9H655 | FLJ22595 | NM_025047 | 287702 | −0.17240792 | Hypothetical protein |
| | | | | | FLJ22595 |
| HSAC018574 | 0 | AK025291 | 306794 | −0.16937687 | Homo sapiens cDNA: |
| | | | | | FLJ21638 fis, clone |
| | | | | | COL08269 |
| Q9UK76 | HN1 | NM_016185 | 109706 | 0.16129563 | Hematological and |
| | | | | | neurological expressed 1 |
| INI2_HUMAN | G1P3 | NM_022873 | 265827 | −0.15984033 | Interferon, alpha- |
| | | | | | inducible protein (clone |
| | | | | | IFI-6-16) |
| HRG_HUMAN | HRG | NM_000412 | 1498 | 0.15777333 | Histidine-rich |
| | | | | | glycoprotein |
| Q9ULD2 | ATIP1 | AB033114 | 7946 | −0.15367977 | AT2 receptor- |
| | | | | | interacting protein 1 |
| TRBM_HUMAN | THBD | NM_000361 | 2030 | −0.1417714 | Thrombomodulin |
| HSAC015082 | 0 | AK023326 | 210844 | −0.13617964 | Homo sapiens cDNA |
| | | | | | FLJ13264 fis, clone |
| | | | | | OVARC1000936, |
| | | | | | weakly similar to COAT |
| | | | | | PROTEIN GP37 |
| HSAC002372 | 0 | AK023269 | 14355 | 0.13539305 | Homo sapiens cDNA |
| | | | | | FLJ13207 fis, clone |
| | | | | | NT2RP4000023 |
| ITB4_HUMAN | ITGB4 | NM_000213 | 85266 | 0.12885959 | Integrin, beta 4 |
| HSAC013491 | 0 | AL157491 | 145211 | −0.11058947 | Homo sapiens mRNA; |
| | | | | | cDNA |
| | | | | | DKFZp434K1111 (from |
| | | | | | clone |
| | | | | | DKFZp434K1111) |
| Q8TAD5 | SPANXB1 | NM_032461 | 293266 | 0.08476128 | SPANX family, |
| | | | | | member B1 |
| Q96SV0 | FLJ14621 | NM_032811 | 10056 | −0.08227502 | Hypothetical protein |
| | | | | | FLJ14621 |
| Q9ULE7 | KIAA1273 | AB033099 | 23413 | −0.05093065 | KIAA1273 protein |

TABLE 4
List of 179 genes with strong predictive value

| | | | | N+ | |
|---|---|---|---|---|---|
| UniProt | Gene_symbol | GB_accession | UniGene_ID | correlation | Gene |
| Q9NZQ6 | COL5A3 | NM_015719 | 235368 | 0.57481098 | Collagen, type V, alpha 3 |
| FINC_HUMAN | FN1 | NM_002026 | 287820 | 0.55126846 | Fibronectin 1 |
| FSL1_HUMAN | FSTL1 | NM_007085 | 296267 | 0.53452734 | Follistatin-like 1 |
| ER22_HUMAN | KDELR2 | NM_006854 | 118778 | 0.52986731 | KDEL (Lys-Asp-Glu- |
| | | | | | Leu) endoplasmic |
| | | | | | reticulum protein |
| | | | | | retention receptor 2 |
| PCO1_HUMAN | PCOLCE | NM_002593 | 202097 | 0.52672595 | Procollagen C- |
| | | | | | endopeptidase |
| | | | | | enhancer |
| SPRC_HUMAN | SPARC | NM_003118 | 111779 | 0.51922531 | Secreted protein, |
| | | | | | acidic, cysteine-rich |
| | | | | | (osteonectin) |
| NC5R_HUMAN | DIA1 | NM_007326 | 274464 | 0.51482855 | Diaphorase (NADH) |
| | | | | | (cytochrome b-5 |
| | | | | | reductase) |
| SEPR_HUMAN | FAP | NM_004460 | 418 | 0.51466336 | Fibroblast activation |
| | | | | | protein, alpha |
| HSAC013564 | COL5A1 | NM_000093 | 146428 | 0.51225806 | Collagen, type V, alpha |
| | | | | | 1 |
| HSAC009848 | ZFP93 | NM_004234 | 298089 | 0.50989391 | Zinc finger protein 93 |
| | | | | | homolog (mouse) |
| PGCV_HUMAN | CSPG2 | U16306 | 81800 | 0.50906177 | Chondroitin sulfate |
| | | | | | proteoglycan 2 |
| | | | | | (versican) |
| Q14521 | LLGL2 | NM_004524 | 3123 | −0.50695888 | Lethal giant larvae |
| | | | | | homolog 2 (Drosophila) |
| CA14_HUMAN | COL4A1 | NM_001845 | 119129 | 0.50664753 | Collagen, type IV, |
| | | | | | alpha 1 |
| HSAC014709 | TEM1 | NM_020404 | 195727 | 0.50647843 | Tumor endothelial |
| | | | | | marker 1 precursor |
| UROK_HUMAN | PLAU | NM_002658 | 77274 | 0.50439834 | Plasminogen activator, |
| | | | | | urokinase |
| Q8N6P7 | IL22R | NM_021258 | 110915 | −0.49928496 | Interleukin 22 receptor |
| LEG1_HUMAN | LGALS1 | NM_002305 | 227751 | 0.48740219 | Lectin, galactoside- |
| | | | | | binding, soluble, 1 |
| | | | | | (galectin 1) |
| CA25_HUMAN | COL5A2 | NM_000393 | 82985 | 0.48675513 | Collagen, type V, alpha |
| | | | | | 2 |
| CA13_HUMAN | COL3A1 | NM_000090 | 119571 | 0.48459913 | Collagen, type III, |
| | | | | | alpha 1 (Ehlers-Danlos |
| | | | | | syndrome type IV, |
| | | | | | autosomal dominant) |
| Q9BYD5 | LOC84518 | NM_032488 | 148590 | −0.48449285 | Protein related with |
| | | | | | psoriasis |
| BGH3_HUMAN | TGFBI | NM_000358 | 118787 | 0.48290139 | Transforming growth |
| | | | | | factor, beta-induced, |
| | | | | | 68 kD |
| CQT6_HUMAN | CTRP6 | NM_031910 | 22011 | 0.47978614 | Complement-c1q tumor |
| | | | | | necrosis factor-related |
| | | | | | protein 6 |
| AD12_HUMAN | ADAM12 | NM_003474 | 8850 | 0.47905457 | A disintegrin and |
| | | | | | metalloproteinase |
| | | | | | domain 12 (meltrin |
| | | | | | alpha) |
| Q8N3N2 | FLJ11196 | NM_018357 | 6166 | 0.47772636 | Hypothetical protein |
| | | | | | FLJ11196 |
| CATK_HUMAN | CTSK | NM_000396 | 83942 | 0.47734967 | Cathepsin K |
| | | | | | (pycnodysostosis) |
| Q96DR2 | 0 | AK055031 | 44289 | −0.47659518 | Homo sapiens cDNA |
| | | | | | FLJ30469 fis, clone |
| | | | | | BRAWH1000037, |
| | | | | | weakly similar to |
| | | | | | UROKINASE |
| | | | | | PLASMINOGEN |
| | | | | | ACTIVATO |
| P03996 | ACTA2 | NM_001613 | 195851 | 0.47560995 | Actin, alpha 2, smooth |
| | | | | | muscle, aorta |
| K6A2_HUMAN | 0 | AK027727 | 184581 | 0.47396182 | Homo sapiens cDNA |
| | | | | | FLJ14821 fis, clone |
| | | | | | OVARC1000556, |
| | | | | | highly similar to |
| | | | | | RIBOSOMAL |
| | | | | | PROTEIN S6 KINASE |
| | | | | | II |
| Q9HBB0 | THY1 | AK057865 | 125359 | 0.4727028 | Thy-1 cell surface |
| | | | | | antigen |
| TM29_HUMAN | TRIM29 | NM_012101 | 82237 | −0.47250994 | Tripartite motif- |
| | | | | | containing 29 |
| TIM2_HUMAN | 0 | AL110197 | 6441 | 0.47199573 | Homo sapiens mRNA; |
| | | | | | cDNA DKFZp586J021 |
| | | | | | (from clone |
| | | | | | DKFZp586J021) |
| MM02_HUMAN | MMP2 | NM_004530 | 111301 | 0.47026319 | Matrix |
| | | | | | metalloproteinase 2 |
| | | | | | (gelatinase A, 72 kD |
| | | | | | gelatinase, 72 kD type |
| | | | | | IV collagenase) |
| MCA2_HUMAN | JTV1 | NM_006303 | 301613 | 0.46964142 | JTV1 gene |
| CA16_HUMAN | COL6A1 | NM_001848 | 108885 | 0.46942561 | Collagen, type VI, |
| | | | | | alpha 1 |
| EVA1_HUMAN | EVA1 | AF275945 | 116651 | −0.4689165 | Epithelial V-like antigen |
| | | | | | 1 |
| CA21_HUMAN | COL1A2 | NM_000089 | 179573 | 0.46739585 | Collagen, type I, alpha |
| | | | | | 2 |
| CA36_HUMAN | COL6A3 | NM_004369 | 80988 | 0.46507048 | Collagen, type VI, |
| | | | | | alpha 3 |
| OPN3_HUMAN | OPN3 | NM_014322 | 279926 | 0.46101056 | Opsin 3 |
| | | | | | (encephalopsin, |
| | | | | | panopsin) |
| Q9UBG0 | KIAA0709 | NM_006039 | 7835 | 0.46012143 | Endocytic receptor |
| | | | | | (macrophage mannose |
| | | | | | receptor family) |
| TPM2_HUMAN | TPM2 | NM_003289 | 300772 | 0.46003075 | Tropomyosin 2 (beta) |
| INVO_HUMAN | IVL | NM_005547 | 157091 | −0.45860578 | Involucrin |
| O88386 | RAB10 | NM_016131 | 236494 | −0.45006586 | RAB10, member RAS |
| | | | | | oncogene family |
| PEPL_HUMAN | PPL | NM_002705 | 74304 | −0.44874989 | Periplakin |
| HSAC002603 | FLJ11036 | NM_018306 | 16740 | −0.44841185 | Hypothetical protein |
| | | | | | FLJ11036 |
| TNR5_HUMAN | TNFRSF5 | NM_001250 | 25648 | −0.44547341 | Tumor necrosis factor |
| | | | | | receptor superfamily, |
| | | | | | member 5 |
| FRIH_HUMAN | FTH1 | AK054816 | 62954 | 0.4396982 | Ferritin, heavy |
| | | | | | polypeptide 1 |
| P4H2_HUMAN | P4HA2 | NM_004199 | 3622 | 0.42478412 | Procollagen-proline, 2- |
| | | | | | oxoglutarate 4- |
| | | | | | dioxygenase (proline 4- |
| | | | | | hydroxylase), alpha |
| | | | | | polypeptide II |
| P09526 | RAP1B | NM_015646 | 156764 | 0.42234208 | RAP1B, member of |
| | | | | | RAS oncogene family |
| PS23_HUMAN | SPUVE | NM_007173 | 25338 | 0.42080095 | Protease, serine, 23 |
| HSAC011159 | 0 | AF009267 | 102238 | −0.46560322 | Homo sapiens clone |
| | | | | | FBA1 Cri-du-chat |
| | | | | | region mRNA |
| SDC2_HUMAN | SDC2 | J04621 | 1501 | 0.45455196 | Syndecan 2 (heparan |
| | | | | | sulfate proteoglycan 1, |
| | | | | | cell surface-associated, |
| | | | | | fibroglycan) |
| HSAC013320 | 0 | AL162069 | 140978 | −0.44362782 | Homo sapiens mRNA; |
| | | | | | cDNA DKFZp762H106 |
| | | | | | (from clone |
| | | | | | DKFZp762H106) |
| TAGL_HUMAN | TAGLN | NM_003186 | 75777 | 0.4376112 | Transgelin |
| MM01_HUMAN | MMP1 | NM_002421 | 83169 | 0.4326825 | Matrix |
| | | | | | metalloproteinase 1 |
| | | | | | (interstitial |
| | | | | | collagenase) |
| P05209 | K-ALPHA-1 | NM_006082 | 334842 | 0.42236541 | Tubulin, alpha, |
| | | | | | ubiquitous |
| TSP2_HUMAN | THBS2 | NM_003247 | 108623 | 0.46791583 | Thrombospondin 2 |
| Q8N789 | DKFZP4 | AL137589 | 152149 | −0.43787021 | Hypothetical protein |
| | 34K0410 | | | | DKFZp434K0410 |
| O60335 | KIAA0594 | AB011166 | 103283 | −0.42578156 | KIAA0594 protein |
| P05209 | K-ALPHA-1 | NM_006082 | 334842 | 0.42277793 | Tubulin, alpha, |
| | | | | | ubiquitous |
| TNI3_HUMAN | TNFAIP3 | NM_006290 | 211600 | 0.408128 | Tumor necrosis factor, |
| | | | | | alpha-induced protein 3 |
| FGR1_HUMAN | FGFR1 | NM_023109 | 748 | 0.43553561 | Fibroblast growth factor |
| | | | | | receptor 1 (fms-related |
| | | | | | tyrosine kinase 2, |
| | | | | | Pfeiffer syndrome) |
| CAD2_HUMAN | CDH2 | NM_001792 | 161 | 0.3922152 | Cadherin 2, type 1, N- |
| | | | | | cadherin (neuronal) |
| TCOF_HUMAN | TCOF1 | NM_000356 | 301266 | 0.38956707 | Treacher Collins- |
| | | | | | Franceschetti |
| | | | | | syndrome 1 |
| O14635 | 0 | AF005082 | 113261 | −0.45637623 | Homo sapiens skin- |
| | | | | | specific protein (xp33) |
| | | | | | mRNA, partial cds |
| GLSK_HUMAN | GLS | NM_014905 | 239189 | 0.43214995 | Glutaminase |
| Q9BRJ6 | MGC11257 | NM_032350 | 334368 | 0.42954566 | Hypothetical protein |
| | | | | | MGC11257 |
| ALK1_HUMAN | SLPI | NM_003064 | 251754 | −0.41872706 | Secretory leukocyte |
| | | | | | protease inhibitor |
| | | | | | (antileukoproteinase) |
| AQP3_HUMAN | AQP3 | NM_004925 | 234642 | −0.42891866 | Aquaporin 3 |
| SPIB_HUMAN | SPIB | NM_003121 | 192861 | −0.41021146 | Spi-B transcription |
| | | | | | factor (Spi-1/PU.1 |
| | | | | | related) |
| P05209 | K-ALPHA-1 | NM_006082 | 334842 | 0.41796901 | Tubulin, alpha, |
| | | | | | ubiquitous |
| DRG1_HUMAN | DRG1 | NM_004147 | 115242 | 0.41879372 | Developmentally |
| | | | | | regulated GTP binding |
| | | | | | protein 1 |
| PHMX_HUMAN | PHEMX | NM_005705 | 271954 | 0.38728757 | Pan-hematopoietic |
| | | | | | expression |
| P05209 | K-ALPHA-1 | NM_006082 | 334842 | 0.41741385 | Tubulin, alpha, |
| | | | | | ubiquitous |
| HSAC018335 | 0 | AL137428 | 306459 | −0.45404252 | Homo sapiens mRNA; |
| | | | | | cDNA |
| | | | | | DKFZp761N1323 (from |
| | | | | | clone |
| | | | | | DKFZp761N1323) |
| POSN_HUMAN | OSF-2 | NM_006475 | 136348 | 0.42814786 | Osteoblast specific |
| | | | | | factor 2 (fasciclin I-like) |
| DHC3_HUMAN | CBR3 | NM_001236 | 154510 | −0.48089013 | Carbonyl reductase 3 |
| NCR2_HUMAN | NCOR2 | NM_006312 | 287994 | 0.42024512 | Nuclear receptor co- |
| | | | | | repressor 2 |
| HSAC015262 | 0 | AK021531 | 224398 | 0.43632187 | Homo sapiens cDNA |
| | | | | | FLJ11469 fis, clone |
| | | | | | HEMBA1001658 |
| Q14113 | AEBP1 | NM_001129 | 118397 | 0.42087649 | AE binding protein 1 |
| TBX2_HUMAN | TBX2 | AK001031 | 322856 | 0.41381789 | T-box 2 |
| CRF_HUMAN | CRH | NM_000756 | 75294 | −0.41353804 | Corticotropin releasing |
| | | | | | hormone |
| Q9NUJ7 | FLJ11323 | NM_018390 | 25625 | −0.43842923 | Hypothetical protein |
| | | | | | FLJ11323 |
| Q96DU1 | AKAP2 | AJ303079 | 42322 | −0.44106809 | A kinase (PRKA) |
| | | | | | anchor protein 2 |
| P05209 | K-ALPHA-1 | NM_006082 | 334842 | 0.40736744 | Tubulin, alpha, |
| | | | | | ubiquitous |
| Q969Y7 | MGC4677 | NM_052871 | 337986 | 0.39455354 | Hypothetical protein |
| | | | | | MGC4677 |
| Q9BXY6 | FLJ13962 | NM_024862 | 330407 | −0.44327759 | Hypothetical protein |
| | | | | | FLJ13962 |
| K1CW_HUMAN | HAIK1 | NM_015515 | 9029 | −0.42594883 | Type I intermediate |
| | | | | | filament cytokeratin |
| HSAC019114 | FLJ22622 | NM_025151 | 324841 | −0.4331912 | Hypothetical protein |
| | | | | | FLJ22622 |
| PGS2_HUMAN | DCN | NM_001920 | 76152 | 0.39882715 | Decorin |
| DCOP_HUMAN | ODC-p | NM_052998 | 91681 | −0.41609162 | Ornithine |
| | | | | | decarboxylase-like |
| | | | | | protein |
| HSAC020747 | 0 | AK056828 | 350748 | −0.40822563 | Homo sapiens cDNA |
| | | | | | FLJ32266 fis, clone |
| | | | | | PROST1000419 |
| P05209 | K-ALPHA-1 | NM_006082 | 334842 | 0.39997295 | Tubulin, alpha, |
| | | | | | ubiquitous |
| Q96F00 | 0 | AK025719 | 251664 | 0.39457176 | Homo sapiens cDNA: |
| | | | | | FLJ22066 fis, clone |
| | | | | | HEP10611 |
| ISK5_HUMAN | SPINK5 | NM_006846 | 331555 | −0.43770884 | Serine protease |
| | | | | | inhibitor, Kazal type, 5 |
| GFR1_HUMAN | GFRA1 | NM_005264 | 105445 | 0.37859516 | GDNF family receptor |
| | | | | | alpha 1 |
| AAF24516 | NUDEL | NM_030808 | 3850 | −0.40726277 | LIS1-interacting protein |
| | | | | | NUDEL; |
| | | | | | endooligopeptidase A |
| P05209 | K-ALPHA-1 | NM_006082 | 334842 | 0.39594293 | Tubulin, alpha, |
| | | | | | ubiquitous |
| O60836 | T1A-2 | NM_013317 | 135150 | 0.37127534 | Lung type-I cell |
| | | | | | membrane-associated |
| | | | | | glycoprotein |
| KLKA_HUMAN | KLK10 | NM_002776 | 69423 | −0.40584943 | Kallikrein 10 |
| Q96KC3 | MGC3047 | NM_032348 | 59384 | 0.39922401 | Hypothetical protein |
| | | | | | MGC3047 |
| O95274 | C4.4A | NM_014400 | 11950 | −0.39942513 | GPI-anchored |
| | | | | | metastasis-associated |
| | | | | | protein homolog |
| HSAC015726 | SERPINB13 | AJ001696 | 241407 | −0.41273549 | Serine (or cysteine) |
| | | | | | proteinase inhibitor, |
| | | | | | clade B (ovalbumin), |
| | | | | | member 13 |
| SEN7_HUMAN | SENP7 | AL136599 | 30443 | −0.37916215 | Sentrin/SUMO-specific |
| | | | | | protease |
| HSAC015968 | RPL34P2 | AL049714 | 247903 | −0.39984188 | Ribosomal protein L34 |
| | | | | | pseudogene 2 |
| IM8B_HUMAN | TIMM8B | NM_012459 | 279915 | −0.39646264 | Translocase of inner |
| | | | | | mitochondrial |
| | | | | | membrane 8 homolog |
| | | | | | B (yeast) |
| P05209 | K-ALPHA-1 | NM_006082 | 334842 | 0.39265415 | Tubulin, alpha, |
| | | | | | ubiquitous |
| CYTB_HUMAN | CSTB | NM_000100 | 695 | −0.39593616 | Cystatin B (stefin B) |
| MMDP_HUMAN | MMD | NM_012329 | 79889 | 0.35585533 | Monocyte to |
| | | | | | macrophage |
| | | | | | differentiation- |
| | | | | | associated |
| Q9H5J1 | PREI3 | NM_015387 | 107942 | −0.39957843 | Preimplantation protein |
| | | | | | 3 |
| Q9Y283 | INVS | NM_014425 | 104715 | 0.38165914 | Inversin |
| S107_HUMAN | S100A7 | NM_002963 | 112408 | −0.38002947 | S100 calcium binding |
| | | | | | protein A7 (psoriasin 1) |
| SR19_HUMAN | SRP19 | NM_003135 | 2943 | −0.39927981 | Signal recognition |
| | | | | | particle 19 kD |
| MA17_HUMAN | DD96 | NM_005764 | 271473 | −0.38289729 | Epithelial protein up- |
| | | | | | regulated in carcinoma, |
| | | | | | membrane associated |
| | | | | | protein 17 |
| O75943 | RAD17 | NM_002873 | 16184 | −0.38971266 | RAD17 homolog (S. |
| | | | | | pombe) |
| THA_HUMAN | THRA | NM_003250 | 724 | 0.38475204 | Thyroid hormone |
| | | | | | receptor, alpha |
| | | | | | (erythroblastic |
| | | | | | leukemia viral (v-erb-a) |
| | | | | | oncogene homolog, |
| | | | | | avian) |
| HSAC008967 | 0 | AK021982 | 287465 | 0.38611307 | Homo sapiens cDNA |
| | | | | | FLJ11920 fis, clone |
| | | | | | HEMBB1000312 |
| TFE2_HUMAN | TCF3 | M31523 | 101047 | 0.38678059 | Transcription factor 3 |
| | | | | | (E2A immunoglobulin |
| | | | | | enhancer binding |
| | | | | | factors E12/E47) |
| SUL2_HUMAN | KIAA1247 | AB033073 | 43857 | 0.39182636 | Similar to glucosamine- |
| | | | | | 6-sulfatases |
| HRA3_HUMAN | HTRA3 | AY040094 | 60440 | 0.38043383 | Serine protease |
| | | | | | HTRA3 |
| CN4A_HUMAN | PDE4A | NM_006202 | 89901 | 0.36750244 | Phosphodiesterase 4A, |
| | | | | | cAMP-specific |
| | | | | | (phosphodiesterase E2 |
| | | | | | dunce homolog, |
| | | | | | Drosophila) |
| LTB2_HUMAN | LTBP2 | NM_000428 | 83337 | 0.36424793 | Latent transforming |
| | | | | | growth factor beta |
| | | | | | binding protein 2 |
| CSF2_HUMAN | CSF2 | NM_000758 | 1349 | 0.34759785 | Colony stimulating |
| | | | | | factor 2 (granulocyte- |
| | | | | | macrophage) |
| S109_HUMAN | S100A9 | NM_002965 | 112405 | −0.38115533 | S100 calcium binding |
| | | | | | protein A9 (calgranulin |
| | | | | | B) |
| MAL2_HUMAN | MAL2 | NM_052886 | 76550 | −0.37756452 | Mal, T-cell |
| | | | | | differentiation protein 2 |
| HSAC004288 | LANO | NM_018214 | 35091 | −0.39020801 | LAP (leucine-rich |
| | | | | | repeats and PDZ) and |
| | | | | | no PDZ protein |
| P05209 | K-ALPHA-1 | NM_006082 | 334842 | 0.37816007 | Tubulin, alpha, |
| | | | | | ubiquitous |
| EMP3_HUMAN | EMP3 | NM_001425 | 9999 | 0.37961495 | Epithelial membrane |
| | | | | | protein 3 |
| LUM_HUMAN | LUM | NM_002345 | 79914 | 0.36091717 | Lumican |
| Q8NC43 | FLJ23091 | NM_024911 | 250746 | 0.4002659 | Hypothetical protein |
| | | | | | FLJ23091 |
| HRA1_HUMAN | PRSS11 | NM_002775 | 75111 | 0.38314991 | Protease, serine, 11 |
| | | | | | (IGF binding) |
| CAH6_HUMAN | CA6 | NM_001215 | 100322 | 0.38495881 | Carbonic anhydrase VI |
| SCGF_HUMAN | SCGF | NM_002975 | 105927 | 0.38520465 | Stem cell growth factor; |
| | | | | | lymphocyte secreted |
| | | | | | C-type lectin |
| CALD_HUMAN | CALD1 | NM_033138 | 325474 | 0.36017333 | Caldesmon 1 |
| SYH_HUMAN | HARS | NM_002109 | 77798 | 0.34476889 | Histidyl-tRNA |
| | | | | | synthetase |
| Q8IXQ7 | LABH1 | NM_032604 | 98608 | 0.36012503 | Lung alpha/beta |
| | | | | | hydrolase 1 |
| WEE1_HUMAN | WEE1 | X62048 | 75188 | −0.38967133 | WEE1+ homolog (S. |
| | | | | | pombe) |
| Q9H0B8 | DKFZP434B044 | NM_031476 | 262958 | 0.36902739 | Hypothetical protein |
| | | | | | DKFZp434B044 |
| M1B1_HUMAN | MAN1B1 | NM_016219 | 279881 | 0.37430439 | Mannosidase, alpha, |
| | | | | | class 1B, member 1 |
| FBX8_HUMAN | FBXO8 | NM_012180 | 76917 | −0.37436238 | F-box only protein 8 |
| SM3C_HUMAN | SEMA3C | NM_006379 | 171921 | 0.35697551 | Sema domain, |
| | | | | | immunoglobulin |
| | | | | | domain (Ig), short basic |
| | | | | | domain, secreted, |
| | | | | | (semaphorin) 3C |
| RB25_HUMAN | CATX-8 | NM_020387 | 150826 | −0.38816294 | CATX-8 protein |
| ROL_HUMAN | HNRPL | NM_001533 | 2730 | 0.34146766 | Heterogeneous nuclear |
| | | | | | ribonucleoprotein L |
| FX37_HUMAN | MGC11279 | NM_024326 | 10915 | −0.38119046 | Hypothetical protein |
| | | | | | MGC11279 |
| HSAC003262 | KIAA0350 | AB002348 | 23263 | −0.39564135 | KIAA0350 protein |
| P05209 | K-ALPHA-1 | NM_006082 | 334842 | 0.37391308 | Tubulin, alpha, |
| | | | | | ubiquitous |
| BTE4_HUMAN | KLF16 | NM_031918 | 303194 | 0.40092333 | Kruppel-like factor 16 |
| MK_HUMAN | MDK | NM_002391 | 82045 | −0.38900237 | Midkine (neurite |
| | | | | | growth-promoting |
| | | | | | factor 2) |
| Q9NRD9 | DUOX1 | NM_017434 | 272813 | −0.3913498 | Dual oxidase 1 |
| P05209 | K-ALPHA-1 | NM_006082 | 334842 | 0.36936704 | Tubulin, alpha, |
| | | | | | ubiquitous |
| Z185_HUMAN | ZNF185 | NM_007150 | 16622 | −0.36378851 | Zinc finger protein 185 |
| | | | | | (LIM domain) |
| TBG2_HUMAN | TUBG2 | NM_016437 | 279669 | 0.34203519 | Tubulin, gamma 2 |
| AAKC_HUMAN | PRKAB2 | NM_005399 | 50732 | −0.35738949 | Protein kinase, AMP- |
| | | | | | activated, beta 2 non- |
| | | | | | catalytic subunit |
| HSAC006508 | COL18A1 | AF018081 | 78409 | 0.37705859 | Collagen, type XVIII, |
| | | | | | alpha 1 |
| Q9BSY6 | ZD52F10 | NM_033317 | 32343 | −0.37419257 | Hypothetical gene |
| | | | | | ZD52F10 |
| SOX4_HUMAN | 0 | AJ420500 | 351928 | −0.41521785 | Homo sapiens mRNA |
| | | | | | full length insert cDNA |
| | | | | | clone EUROIMAGE |
| | | | | | 1977059 |
| P05209 | K-ALPHA-1 | NM_006082 | 334842 | 0.37198391 | Tubulin, alpha, |
| | | | | | ubiquitous |
| MIC2_HUMAN | MIC2 | NM_002414 | 177543 | 0.36316608 | Antigen identified by |
| | | | | | monoclonal antibodies |
| | | | | | 12E7, F21 and O13 |
| VWF_HUMAN | VWF | NM_000552 | 110802 | −0.36187757 | Von Willebrand factor |
| MFA5_HUMAN | MAGP2 | NM_003480 | 300946 | 0.34145531 | Microfibril-associated |
| | | | | | glycoprotein-2 |
| ELAF_HUMAN | PI3 | NM_002638 | 112341 | −0.39367703 | Protease inhibitor 3, |
| | | | | | skin-derived (SKALP) |
| WD13_HUMAN | WDR13 | NM_017883 | 12142 | 0.35576479 | WD repeat domain 13 |
| PCB1_HUMAN | PCBP1 | NM_006196 | 2853 | −0.35199657 | Poly(rC) binding |
| | | | | | protein 1 |
| DYHC_HUMAN | DNCH1 | AB002323 | 7720 | 0.36908919 | Dynein, cytoplasmic, |
| | | | | | heavy polypeptide 1 |
| Q8WUB2 | HSU79274 | NM_013300 | 150555 | −0.41095099 | Protein predicted by |
| | | | | | clone 23733 |
| Q96N74 | PGLYRP | NM_052890 | 282244 | −0.37818832 | Peptidoglycan |
| | | | | | recognition protein L |
| | | | | | precursor |
| HSAC018816 | 0 | AK055723 | 310919 | −0.37825237 | Homo sapiens cDNA |
| | | | | | FLJ31161 fis, clone |
| | | | | | KIDNE1000028 |
| PPL2_HUMAN | PPIL2 | NM_014337 | 93523 | −0.34926253 | Peptidylprolyl |
| | | | | | isomerase |
| | | | | | (cyclophilin)-like 2 |
| HSAC015090 | 0 | AK055294 | 211132 | 0.34738745 | Homo sapiens cDNA |
| | | | | | FLJ30732 fis, clone |
| | | | | | FEBRA2000126, |
| | | | | | weakly similar to Mus |
| | | | | | musculus PDZ domain |
| | | | | | actin |
| P05209 | K-ALPHA-1 | NM_006082 | 334842 | 0.36404497 | Tubulin, alpha, |
| | | | | | ubiquitous |
| DSG3_HUMAN | DSG3 | NM_001944 | 1925 | −0.35916779 | Desmoglein 3 |
| | | | | | (pemphigus vulgaris |
| | | | | | antigen) |
| CTGF_HUMAN | CTGF | NM_001901 | 75511 | 0.36449331 | Connective tissue |
| | | | | | growth factor |
| Q96BW1 | 0 | AK056354 | 91612 | 0.34964326 | Homo sapiens, clone |
| | | | | | MGC: 23937 |
| | | | | | IMAGE: 3930177, |
| | | | | | mRNA, complete cds |
| P05209 | K-ALPHA-1 | NM_006082 | 334842 | 0.36440521 | Tubulin, alpha, |
| | | | | | ubiquitous |
| HSAC020349 | 0 | BC014584 | 348710 | 0.33020586 | Homo sapiens, clone |
| | | | | | IMAGE: 4047062, |
| | | | | | mRNA |
| G3P2_HUMAN | GAPD | NM_002046 | 169476 | 0.36112372 | Glyceraldehyde-3- |
| | | | | | phosphate |
| | | | | | dehydrogenase |
| DES1_HUMAN | DESC1 | NM_014058 | 201877 | −0.37199135 | DESC1 protein |
| PAI2_HUMAN | SERPINB2 | NM_002575 | 75716 | −0.39418824 | Serine (or cysteine) |
| | | | | | proteinase inhibitor, |
| | | | | | clade B (ovalbumin), |
| | | | | | member 2 |
| PYG2_HUMAN | 0 | BC006132 | 172084 | 0.36848424 | Homo sapiens, clone |
| | | | | | IMAGE: 3627860, |
| | | | | | mRNA, partial cds |
| CA17_HUMAN | COL7A1 | NM_000094 | 1640 | 0.35840992 | Collagen, type VII, |
| | | | | | alpha 1 (epidermolysis |
| | | | | | bullosa, dystrophic, |
| | | | | | dominant and |
| | | | | | recessive) |
| Q8NG54 | FLJ21212 | NM_024642 | 47099 | −0.35274616 | Hypothetical protein |
| | | | | | FLJ21212 |

TABLE 5
List of 61 genes with highest predictive value

| | | | | N+ | |
|---|---|---|---|---|---|
| UniProt | Gene_symbol | GB_accession | UniGene_ID | correlation | Gene |
| FSL1_HUMAN | FSTL1 | NM_007085 | 296267 | 0.53452734 | Follistatin-like 1 |
| ER22_HUMAN | KDELR2 | NM_006854 | 118778 | 0.52986731 | KDEL (Lys-Asp-Glu- |
| | | | | | Leu) endoplasmic |
| | | | | | reticulum protein |
| | | | | | retention receptor 2 |
| PCO1_HUMAN | PCOLCE | NM_002593 | 202097 | 0.52672595 | Procollagen C- |
| | | | | | endopeptidase |
| | | | | | enhancer |
| SPRC_HUMAN | SPARC | NM_003118 | 111779 | 0.51922531 | Secreted protein, |
| | | | | | acidic, cysteine-rich |
| | | | | | (osteonectin) |
| NC5R_HUMAN | DIA1 | NM_007326 | 274464 | 0.51482855 | Diaphorase (NADH) |
| | | | | | (cytochrome b-5 |
| | | | | | reductase) |
| HSAC009848 | ZFP93 | NM_004234 | 298089 | 0.50989391 | Zinc finger protein 93 |
| | | | | | homolog (mouse) |
| Q14521 | LLGL2 | NM_004524 | 3123 | −0.50695888 | Lethal giant larvae |
| | | | | | homolog 2 |
| | | | | | (Drosophila) |
| Q8N6P7 | IL22R | NM_021258 | 110915 | −0.49928496 | Interleukin 22 |
| | | | | | receptor |
| LEG1_HUMAN | LGALS1 | NM_002305 | 227751 | 0.48740219 | Lectin, galactoside- |
| | | | | | binding, soluble, 1 |
| | | | | | (galectin 1) |
| Q9BYD5 | LOC84518 | NM_032488 | 148590 | −0.48449285 | Protein related with |
| | | | | | psoriasis |
| CQT6_HUMAN | CTRP6 | NM_031910 | 22011 | 0.47978614 | Complement-c1q |
| | | | | | tumor necrosis factor- |
| | | | | | related protein 6 |
| AD12_HUMAN | ADAM12 | NM_003474 | 8850 | 0.47905457 | A disintegrin and |
| | | | | | metalloproteinase |
| | | | | | domain 12 (meltrin |
| | | | | | alpha) |
| Q8N3N2 | FLJ11196 | NM_018357 | 6166 | 0.47772636 | Hypothetical protein |
| | | | | | FLJ11196 |
| Q96DR2 | | AK055031 | 44289 | −0.47659518 | Homo sapiens cDNA |
| | | | | | FLJ30469 fis, clone |
| | | | | | BRAWH1000037, |
| | | | | | weakly similar to |
| | | | | | UROKINASE |
| | | | | | PLASMINOGEN |
| | | | | | ACTIVATO |
| P03996 | ACTA2 | NM_001613 | 195851 | 0.47560995 | Actin, alpha 2, |
| | | | | | smooth muscle, aorta |
| K6A2_HUMAN | | AK027727 | 184581 | 0.47396182 | Homo sapiens cDNA |
| | | | | | FLJ14821 fis, clone |
| | | | | | OVARC1000556, |
| | | | | | highly similar to |
| | | | | | RIBOSOMAL |
| | | | | | PROTEIN S6 |
| | | | | | KINASE II |
| TM29_HUMAN | TRIM29 | NM_012101 | 82237 | −0.47250994 | Tripartite motif- |
| | | | | | containing 29 |
| TIM2_HUMAN | | AL110197 | 6441 | 0.47199573 | Homo sapiens |
| | | | | | mRNA; cDNA |
| | | | | | DKFZp586J021 (from |
| | | | | | clone |
| | | | | | DKFZp586J021) |
| MCA2_HUMAN | JTV1 | NM_006303 | 301613 | 0.46964142 | JTV1 gene |
| OPN3_HUMAN | OPN3 | NM_014322 | 279926 | 0.46101056 | Opsin 3 |
| | | | | | (encephalopsin, |
| | | | | | panopsin) |
| Q9UBG0 | KIAA0709 | NM_006039 | 7835 | 0.46012143 | Endocytic receptor |
| | | | | | (macrophage |
| | | | | | mannose receptor |
| | | | | | family) |
| TPM2_HUMAN | TPM2 | NM_003289 | 300772 | 0.46003075 | Tropomyosin 2 (beta) |
| INVO_HUMAN | IVL | NM_005547 | 157091 | −0.45860578 | Involucrin |
| PEPL_HUMAN | PPL | NM_002705 | 74304 | −0.44874989 | Periplakin |
| HSAC002603 | FLJ11036 | NM_018306 | 16740 | −0.44841185 | Hypothetical protein |
| | | | | | FLJ11036 |
| TNR5_HUMAN | TNFRSF5 | NM_001250 | 25648 | −0.44547341 | Tumor necrosis factor |
| | | | | | receptor superfamily, |
| | | | | | member 5 |
| FRIH_HUMAN | FTH1 | AK054816 | 62954 | 0.4396982 | Ferritin, heavy |
| | | | | | polypeptide 1 |
| P4H2_HUMAN | P4HA2 | NM_004199 | 3622 | 0.42478412 | Procollagen-proline, |
| | | | | | 2-oxoglutarate 4- |
| | | | | | dioxygenase (proline |
| | | | | | 4-hydroxylase), alpha |
| | | | | | polypeptide II |
| PS23_HUMAN | SPUVE | NM_007173 | 25338 | 0.42080095 | Protease, serine, 23 |
| HSAC011159 | | AF009267 | 102238 | −0.46560322 | Homo sapiens clone |
| | | | | | FBA1 Cri-du-chat |
| | | | | | region mRNA |
| HSAC013320 | | AL162069 | 140978 | −0.44362782 | Homo sapiens |
| | | | | | mRNA; cDNA |
| | | | | | DKFZp762H106 |
| | | | | | (from clone |
| | | | | | DKFZp762H106) |
| Q8N789 | DKFZP434K0410 | AL137589 | 152149 | −0.43787021 | Hypothetical protein |
| | | | | | DKFZp434K0410 |
| O60335 | KIAA0594 | AB011166 | 103283 | −0.42578156 | KIAA0594 protein |
| TCOF_HUMAN | TCOF1 | NM_000356 | 301266 | 0.38956707 | Treacher Collins- |
| | | | | | Franceschetti |
| | | | | | syndrome 1 |
| O14635 | | AF005082 | 113261 | −0.45637623 | Homo sapiens skin- |
| | | | | | specific protein |
| | | | | | (xp33) mRNA, partial |
| | | | | | cds |
| GLSK_HUMAN | GLS | NM_014905 | 239189 | 0.43214995 | Glutaminase |
| Q9BRJ6 | MGC11257 | NM_D32350 | 334368 | 0.42954566 | Hypothetical protein |
| | | | | | MGC11257 |
| AQP3_HUMAN | AQP3 | NM_004925 | 234642 | −0.42891866 | Aquaporin 3 |
| SPIB_HUMAN | SPIB | NM_003121 | 192861 | −0.41021146 | Spi-B transcription |
| | | | | | factor (Spi-1/PU.1 |
| | | | | | related) |
| DRG1_HUMAN | DRG1 | NM_004147 | 115242 | 0.41879372 | Developmentally |
| | | | | | regulated GTP |
| | | | | | binding protein 1 |
| PHMX_HUMAN | PHEMX | NM_005705 | 271954 | 0.38728757 | Pan-hematopoietic |
| | | | | | expression |
| HSAC018335 | | AL137428 | 306459 | −0.45404252 | Homo sapiens |
| | | | | | mRNA; cDNA |
| | | | | | DKFZp761N1323 |
| | | | | | (from clone |
| | | | | | DKFZp761N1323) |
| POSN_HUMAN | OSF-2 | NM_006475 | 136348 | 0.42814786 | Osteoblast specific |
| | | | | | factor 2 (fasciclin I- |
| | | | | | like) |
| DHC3_HUMAN | CBR3 | NM_001236 | 154510 | −0.48089013 | Carbonyl reductase 3 |
| HSAC015262 | | AK021531 | 224398 | 0.43632187 | Homo sapiens cDNA |
| | | | | | FLJ11469 fis, clone |
| | | | | | HEMBA1001658 |
| Q14113 | AEBP1 | NM_001129 | 118397 | 0.42087649 | AE binding protein 1 |
| CRF_HUMAN | CRH | NM_000756 | 75294 | −0.41353804 | Corticotropin |
| | | | | | releasing hormone |
| Q9NUJ7 | FLJ11323 | NM_018390 | 25625 | −0.43842923 | Hypothetical protein |
| | | | | | FLJ11323 |
| Q96DU1 | AKAP2 | AJ303079 | 42322 | −0.44106809 | A kinase (PRKA) |
| | | | | | anchor protein 2 |
| Q969Y7 | MGC4677 | NM_052871 | 337986 | 0.39455354 | Hypothetical protein |
| | | | | | MGC4677 |
| Q9BXY6 | FLJ13962 | NM_024862 | 330407 | −0.44327759 | Hypothetical protein |
| | | | | | FLJ13962 |
| K1CW_HUMAN | HAIK1 | NM_015515 | 9029 | −0.42594883 | Type I intermediate |
| | | | | | filament cytokeratin |
| HSAC019114 | FLJ22622 | NM_025151 | 324841 | −0.4331912 | Hypothetical protein |
| | | | | | FLJ22622 |
| PGS2_HUMAN | DCN | NM_001920 | 76152 | 0.39882715 | Decorin |
| DCOP_HUMAN | ODC-p | NM_052998 | 91681 | −0.41609162 | Ornithine |
| | | | | | decarboxylase-like |
| | | | | | protein |
| HSAC020747 | 0 | AK056828 | 350748 | −0.40822563 | Homo sapiens cDNA |
| | | | | | FLJ32266 fis, clone |
| | | | | | PROST1000419 |
| Q96F00 | 0 | AK025719 | 251664 | 0.39457176 | Homo sapiens cDNA: |
| | | | | | FLJ22066 fis, clone |
| | | | | | HEP10611 |
| ISK5_HUMAN | SPINK5 | NM_006846 | 331555 | −0.43770884 | Serine protease |
| | | | | | inhibitor, Kazal type, |
| | | | | | 5 |
| GFR1_HUMAN | GFRA1 | NM_005264 | 105445 | 0.37859516 | GDNF family receptor |
| | | | | | alpha 1 |
| AAF24516 | NUDEL | NM_030808 | 3850 | −0.40726277 | LIS1-interacting |
| | | | | | protein NUDEL; |
| | | | | | endooligopeptidase A |
| O60836 | T1A-2 | NM_013317 | 135150 | 0.37127534 | Lung type-I cell |
| | | | | | membrane- |
| | | | | | associated |
| | | | | | glycoprotein |
| KLKA_HUMAN | KLK10 | NM_002776 | 69423 | −0.40584943 | Kallikrein 10 |

1. A nucleotide array of maximal 50 nucleotide sequences, for the detection of metastasis in head and neck squamous cell cancer (HNSCC) comprising at least 1 of the elements of Table 5.

2. A nucleotide array for the detection of metastasis in HNSCC having 50 or more of the elements of the genes listed in Table 4.

3. A method to establish reference and control gene expression profiles of patients having had metastasis after HNSCC(N+ group) or no metastasis after HNSCC (N0 group) which method comprises obtaining the gene expression profile from a tumour biopsy sample of each patient, or from pooled samples of each group of patients, on an array comprising the elements set forth in claim 1, or an array of claim 1 thus establishing said reference and control gene expression profiles.

4. A method to predict the presence or risk on occurrence of lymph node metastasis of a HNSCC patient comprising:
(a) obtaining a gene expression profile of nucleic acid isolated from a biopsy sample taken from the patient by assaying it with a nucleotide array comprising the elements set forth in claim 1, or an array of claim 1; and
(b) classifying the expression profile thus obtained as N+ or N0 by comparing said expression profile to expression profiles of a group of HNSCC patients known to have developed metastasis.

5. The method of claim 3, wherein the biopsy sample is a fresh biopsy sample.

6. The method of claim 4, wherein the classifying comprises:
(a) determining the amount of hybridisation of each of the elements of the nucleotide array relative to the amount of hybridisation of each element with a reference sample, and normalizing said amount;
(b) determining whether the expression of the corresponding gene in the biopsy sample is more or less than the expression of the corresponding gene in the reference sample.

7. A method according to claim 4, wherein the expression profile is classified as N+ (high risk of metastasis) or N0 (low or no risk of metastasis) according to the steps of:
a. determining the collective correlation of the classifier/predictor genes or elements present in the expression profile with the average N+ or N0 profile from primary tumors with previously established N-status; and
b. determining the predictive threshold based on the correlation threshold from primary tumors with previously established N-status.

8. A method of claim 4, using the data contained in the E-UMCU-11 dataset in the public microarray database ArrayExpress at web address ebi.ac.uk.arrayexpress, which contains all relevant gene expression measurements for patients with established metastatic status.

9. A method of claim 7, wherein the correlation is determined using the cosine correlation method.

10. A method of claim 6, wherein normalizing of the expression profile is achieved by correcting the expression data for experimental variations with the help of expression data of a control gene or element which is not affected by the tumour state.

11. A method to establish reference and control gene expression profiles of patients having had metastasis after HNSCC(N+ group) or no metastasis after HNSCC (N0 group) which method comprises obtaining the gene expression profile from a tumour biopsy sample of each patient, or from pooled samples of each group of patients, on an array comprising the elements set forth in claim 2, or an array of claim 2 thus establishing said reference and control gene expression profiles.

12. A method to predict the presence or risk on occurrence of lymph node metastasis of a HNSCC patient comprising:
(a) obtaining a gene expression profile of nucleic acid isolated from a biopsy sample taken from the patient by assaying it with a nucleotide array comprising the elements set forth in claim 2, or an array of claim 2; and
(b) classifying the expression profile thus obtained as N+ or N0 by comparing said expression profile to expression profiles of a group of HNSCC patients known to have developed metastasis.

13. The method of claim 12, wherein the classifying comprises:
(a) determining the amount of hybridisation of each of the elements of the nucleotide array relative to the amount of hybridisation of each element with a reference sample, and normalizing said amount;
(b) determining whether the expression of the corresponding gene in the biopsy sample is more or less than the expression of the corresponding gene in the reference sample.

14. A method according to claim 13, wherein the expression profile is classified as N+ (high risk of metastasis) or N0 (low or no risk of metastasis) according to the steps of:
c. determining the collective correlation of the classifier/predictor genes or elements present in the expression profile with the average N+ or N0 profile from primary tumors with previously established N-status; and
d. determining the predictive threshold based on the correlation threshold from primary tumors with previously established N-status.

15. A method of claim 13, wherein normalizing of the expression profile is achieved by correcting the expression data for experimental variations with the help of expression data of a control gene or element which is not affected by the tumour state.


SEQUENCE LISTING (1 sequence)

SEQ ID NO: 1 - protein, 4 aa, Homo sapiens
KDEL